SEQUENCE LISTING (85 sequences)

SEQ ID NO: 1 - protein, 15 aa, artificial sequence
  egg peptide
AEVDCSRFPNATDKE

SEQ ID NO: 2 - protein, 15 aa, artificial sequence
  egg peptide
RFPNATDKEGKDVLV

SEQ ID NO: 3 - protein, 15 aa, artificial sequence
  egg peptide
NATDKEGKDVLVCNK

SEQ ID NO: 4 - protein, 15 aa, artificial sequence
  egg peptide
DKEGKDVLVCNKDLR

SEQ ID NO: 5 - protein, 15 aa, artificial sequence
  egg peptide
VLVCNKDLRPICGTD

SEQ ID NO: 6 - protein, 15 aa, artificial sequence
  egg peptide
CNKDLRPICGTDGVT

SEQ ID NO: 7 - protein, 15 aa, artificial sequence
  egg peptide
DLRPICGTDGVTYTN

SEQ ID NO: 8 - protein, 15 aa, artificial sequence
  egg peptide
PICGTDGVTYTNDCL

SEQ ID NO: 9 - protein, 15 aa, artificial sequence
  egg peptide
YTNDCLLCAYSIEFG

SEQ ID NO: 10 - protein, 15 aa, artificial sequence
  egg peptide
DCLLCAYSIEFGTNI

SEQ ID NO: 11 - protein, 15 aa, artificial sequence
  egg peptide
LCAYSIEFGTNISKE

SEQ ID NO: 12 - protein, 15 aa, artificial sequence
  egg peptide
TNISKEHDGECKETV

SEQ ID NO: 13 - protein, 15 aa, artificial sequence
  egg peptide
ECKETVPMNCSSYAN

SEQ ID NO: 14 - protein, 15 aa, artificial sequence
  egg peptide
PMNCSSYANTTSEDG

SEQ ID NO: 15 - protein, 15 aa, artificial sequence
  egg peptide
YANTTSEDGKVMVLC

SEQ ID NO: 16 - protein, 15 aa, artificial sequence
  egg peptide
EDGKVMVLCNRAFNP

SEQ ID NO: 17 - protein, 15 aa, artificial sequence
  egg peptide
KVMVLCNRAFNPVCG

SEQ ID NO: 18 - protein, 15 aa, artificial sequence
  egg peptide
FNPVCGTDGVTYDNE

SEQ ID NO: 19 - protein, 15 aa, artificial sequence
  egg peptide
VCGTDGVTYDNECLL

SEQ ID NO: 20 - protein, 15 aa, artificial sequence
  egg peptide
TDGVTYDNECLLCAH

SEQ ID NO: 21 - protein, 15 aa, artificial sequence
  egg peptide
CLLCAHKVEQGASVD

SEQ ID NO: 22 - protein, 15 aa, artificial sequence
  egg peptide
KVEQGASVDKRHDGG

SEQ ID NO: 23 - protein, 15 aa, artificial sequence
  egg peptide
SVDKRHDGGCRKELA

SEQ ID NO: 24 - protein, 15 aa, artificial sequence
  egg peptide
KRHDGGCRKELAAVS

SEQ ID NO: 25 - protein, 15 aa, artificial sequence
  egg peptide
DGGCRKELAAVSVDC

SEQ ID NO: 26 - protein, 15 aa, artificial sequence
  egg peptide
ELAAVSVDCSEYPKP

SEQ ID NO: 27 - protein, 15 aa, artificial sequence
  egg peptide
VDCSEYPKPDCTAED

SEQ ID NO: 28 - protein, 15 aa, artificial sequence
  egg peptide
PKPDCTAEDRPLCGS

SEQ ID NO: 29 - protein, 15 aa, artificial sequence
  egg peptide
AEDRPLCGSDNKTYG

SEQ ID NO: 30 - protein, 15 aa, artificial sequence
  egg peptide
CGSDNKTYGNKCNFC

SEQ ID NO: 31 - protein, 15 aa, artificial sequence
  egg peptide
TYGNKCNFCNAVVES

SEQ ID NO: 32 - protein, 15 aa, artificial sequence
  egg peptide
NKCNFCNAVVESNGT

SEQ ID NO: 33 - protein, 15 aa, artificial sequence
  egg peptide
NFCNAVVESNGTLTL

SEQ ID NO: 34 - protein, 15 aa, artificial sequence
  egg peptide
VESNGTLTLSHFGKC

SEQ ID NO: 35 - protein, 15 aa, artificial sequence
  egg peptide
MGSIGAASMEFCFDV

SEQ ID NO: 36 - protein, 15 aa, artificial sequence
  egg peptide
ASMEFCFDVFKELKV

SEQ ID NO: 37 - protein, 15 aa, artificial sequence
  egg peptide
FKELKVHHANENIFY

SEQ ID NO: 38 - protein, 15 aa, artificial sequence
  egg peptide
DKLPGFGDSIEAQCG

SEQ ID NO: 39 - protein, 15 aa, artificial sequence
  egg peptide
SSLRDILNQITKPND

SEQ ID NO: 40 - protein, 15 aa, artificial sequence
  egg peptide
YLQCVKELYRGGLEP

SEQ ID NO: 41 - protein, 15 aa, artificial sequence
  egg peptide
IIRNVLQPSSVDSQT

SEQ ID NO: 42 - protein, 15 aa, artificial sequence
  egg peptide
  misc_feature (5)..(5): Xaa can be any naturally occurring amino acid
SQTAXVLVNAIVFKG

SEQ ID NO: 43 - protein, 15 aa, artificial sequence
  egg peptide
LVNAIVFKGLWEKAF

SEQ ID NO: 44 - protein, 15 aa, artificial sequence
  egg peptide
KDEDTQAMPFRVTEQ

SEQ ID NO: 45 - protein, 15 aa, artificial sequence
  egg peptide
TEQESKPVQMMYQIG

SEQ ID NO: 46 - protein, 15 aa, artificial sequence
  egg peptide
MMYQIGLFRVASMAS

SEQ ID NO: 47 - protein, 15 aa, artificial sequence
  egg peptide
MASEKMKILELPFAS

SEQ ID NO: 48 - protein, 15 aa, artificial sequence
  egg peptide
LEQLESIINFEKLTE

SEQ ID NO: 49 - protein, 15 aa, artificial sequence
  egg peptide
WTSSNVMEERKIKVY

SEQ ID NO: 50 - protein, 15 aa, artificial sequence
  egg peptide
LPRMKMEEKYNLTSV

SEQ ID NO: 51 - protein, 15 aa, artificial sequence
  egg peptide
LKISQAVHAAHAEIN

SEQ ID NO: 52 - protein, 15 aa, artificial sequence
  egg peptide
EAGREVVGSAEAGVD

SEQ ID NO: 53 - protein, 15 aa, artificial sequence
  egg peptide
EFRADHPFLFCIKHI

SEQ ID NO: 54 - protein, 15 aa, artificial sequence
  egg peptide
AAAMKRHGLDNYRGY

SEQ ID NO: 55 - protein, 15 aa, artificial sequence
  egg peptide
GYSLGNWVCAAKFES

SEQ ID NO: 56 - protein, 15 aa, artificial sequence
  egg peptide
RNTDGSTDYGILQIN

SEQ ID NO: 57 - protein, 15 aa, artificial sequence
  egg peptide
TDYGILQINSRWWCN

SEQ ID NO: 58 - protein, 15 aa, artificial sequence
  egg peptide
WCNDGRTPGSRNLCN

SEQ ID NO: 59 - protein, 15 aa, artificial sequence
  egg peptide
WVAWRNRCKGTDVQA

SEQ ID NO: 60 - protein, 15 aa, artificial sequence
  egg peptide
SSPEEKKCNNLRDLT

SEQ ID NO: 61 - protein, 15 aa, artificial sequence
  egg peptide
LRDLTQQERISLTCV

SEQ ID NO: 62 - protein, 15 aa, artificial sequence
  egg peptide
TIEQKLCRQCKGDPK

SEQ ID NO: 63 - protein, 15 aa, artificial sequence
  egg peptide
DQLTPSPRENRIQWC

SEQ ID NO: 64 - protein, 15 aa, artificial sequence
  egg peptide
VMDYRECNLAEVPTH

SEQ ID NO: 65 - protein, 15 aa, artificial sequence
  egg peptide
YQRPASDVICQEYQD

SEQ ID NO: 66 - protein, 15 aa, artificial sequence
  egg peptide
LRDSYGAMADCCSKA

SEQ ID NO: 67 - protein, 15 aa, artificial sequence
  egg peptide
LEKCCKTDNPAECYA

SEQ ID NO: 68 - protein, 15 aa, artificial sequence
  egg peptide
KQETTPINDNVSQCC

SEQ ID NO: 69 - protein, 15 aa, artificial sequence
  egg peptide
GVDTKYVPPPFNPDM

SEQ ID NO: 70 - protein, 17 aa, artificial sequence
  egg peptide
MVDKCCKQSDINTCFGE

SEQ ID NO: 71 - protein, 15 aa, artificial sequence
  egg peptide
NYSMPANCYHILVQD

SEQ ID NO: 72 - protein, 15 aa, artificial sequence
  egg peptide
VQDCSSELKFLVMMK

SEQ ID NO: 73 - protein, 15 aa, artificial sequence
  egg peptide
CAKGCSATKTTPVTV

SEQ ID NO: 74 - protein, 15 aa, artificial sequence
  egg peptide
CSATKTTPVTVGFHC

SEQ ID NO: 75 - protein, 15 aa, artificial sequence
  egg peptide
FHCLPADSANSLTDK

SEQ ID NO: 76 - protein, 15 aa, artificial sequence
  egg peptide
VDHQSLSREVHINTS

SEQ ID NO: 77 - protein, 15 aa, artificial sequence
  egg peptide
GVCGNNDREKHNELL

SEQ ID NO: 78 - protein, 15 aa, artificial sequence
  egg peptide
SSDKKSASEDVVESV

SEQ ID NO: 79 - protein, 186 aa, artificial sequence
  egg peptide
AEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 80 - protein, 386 aa, artificial sequence
  egg peptide
MGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSP

SEQ ID NO: 81 - protein, 687 aa, artificial sequence
  egg peptide
AAPPKSVIRWCTISSPEEKKCNNLRDLTQQERISLTCVQKATYLDCIKAIANNEADAISLDGGQAFEAGLAPYKLKPIAAEVYEHTEGSTTSYYAVAVVKKGTEFTVNDLQGKTSCHTGLGRSAGWNIPIGTLLHRGAIEWEGIESGSVEQAVAKFFSASCVPGATIEQKLCRQCKGDPKTKCARNAPYSGYSGAFHCLKDGKGDVAFVKHTTVNENAPDQKDEYELLCLDGSRQPVDNYKTCNWARVAAHAVVARDDNKVEDIWSFLSKAQSDFGVDTKSDFHLFGPPGKKDPVLKDLLFKDSAIMLKRVPSLMDSQLYLGFEYYSAIQSMRKDQLTPSPRENRIQWCAVGKDEKSKCDRWSVVSNGDVECTVVDETKDCIIKIMKGEADAVALDGGLVYTAGVCGLVPVMAERYDDESQCSKTDERPASYFAVAVARKDSNVNWNNLKGKKSCHTAVGRTAGWVIPMGLIHNRTGTCNFDEYFSEGCAPGSPPNSRLCQLCQGSGGIPPEKCVASSHEKYFGYTGALRCLVEKGDVAFIQHSTVEENTGGKNKADWAKNLQMDDFELLCTDGRRANVMDYRECNLAEVPTHAVVVRPEKANKIRDLLERQEKRFGVNGSEKSKFMMFESQNKDLLFKDLTKCLFKVREGTTYKEFLGDKFYTVISSLKTCNPSDILQMCSFLEGK

SEQ ID NO: 82 - protein, 129 aa, artificial sequence [sequence omitted]

SEQ ID NO: 83 - protein, 592 aa, artificial sequence
  egg peptide
FARDAEHKSEIAHRYNDLKEETFKAVAMITFAQYLQRCSYEGLSKLVKDVVDLAQKCVANEDAPECSKPLPSIILDEICQVEKLRDSYGAMADCCSKADPERNECFLSFKVSQPDFVQPYQRPASDVICQEYQDNRVSFLGHFIYSVARRHPFLYAPAILSFAVDFEHALQSCCKESDVGACLDTKEIVMREKAKGVSVKQQYFCGILKQFGDRVFQARQLIYLSQKYPKAPFSEVSKFVHDSIGVHKECCEGDMVECMDDMARMMSNLCSQQDVFSGKIKDCCEKPIVERSQCIMEAEFDEKPADLPSLVEKYIEDKEVCKSFEAGHDAFMAEFVYEYSRRHPEFSIQLIMRIAKGYESLLEKCCKTDNPAECYANAQEQLNQHIKETQDVVKTNCDLLHDHGEADFLKSILIRYTKKMPQVPTDLLLETGKKMTTIGTKCCQLGEDRRMACSEGYLSIVIHDTCRKQETTPINDNVSQCCSQLYANRRPCFTAMGVDTKYVPPPFNPDMFSFDEKLCSAPAEEREVGQMKLLINLIKRKPQMTEEQIKTIADGFTAMVDKCCKQSDINTCFGEEGANLIVQSRATLGIGA

SEQ ID NO: 84 - protein, 284 aa, artificial sequence
  egg peptide
AEAPSAVLENLKARCSVSYNKIKTFNEVKFNYSMPANCYHILVQDCSSELKFLVMMKSAGEATNLKAINIKIGSHEIDMHPVNGQVKLLVDGAESPTANISLISAGASLWIHNENQGFALAAPGHGIDKLYFDGKTITIQVPLWMAGKTCGICGKYDAECEQEYRMPNGYLAKNAVSFGHSWILEEAPCRGACKLHRSFVKLEKTVQLAGVDSKCYSTEPVLRCAKGCSATKTTPVTVGFHCLPADSANSLTDKQMKYDQKSEDMQDTVDAHTTCSCENEECST

SEQ ID NO: 85 - protein, 285 aa, artificial sequence
  egg peptide
PEIASQIAQEDQSTCEVSKGDFKTFDRMSFTCSFNKSCNVVVAQDCTEHPKFIITTRKVDHQSLSREVHINTSSANITICPAADSSLLVTCNKESVLSDSGVSEYEKDNIKIYKNGKTVIVEAPIHGLKNVNFDGEILKVTVASWMRGKTCGVCGNNDREKHNELLMPNHKLAHSCSAFVHSWVLLEETCSGGCKLQRRYVKLNRNPTIDGEESTCYSVDPVLKCMKDCTPIEKTSVKVGFHCFPKATAVSLLEWQRSSDKKSASEDVVESVDADIDCTCTGDCS

(19) United States
(12) Patent Application Publication
(10) Pub. No.: US 2024/0218029 A1
(43) Pub. Date: Jul. 4, 2024

(54) Peptides And Methods For Detecting Egg Allergies

(71) Applicants: AllerGenis, Inc., Wynnewood, PA (US); ICAHN School of Medicine at Mount Sinai, New York, NY (US)

(72) Inventors: Robert C. Getts, Wynnewood, PA (US); Hugh A. Sampson, New York, NY (US); Maria Suprun, New York, NY (US)

(21) Appl. No.: 18/289,181

(22) PCT Filed: May 6, 2022
(86) PCT No.: PCT/US22/28028

§ 371 Date: Nov. 1, 2023

Related U.S. Application Data
(60) Provisional application No. 63/185,383, filed on May 7, 2021.

(51) Int. Cl. C07K 14/465; A61K 38/00; C07K 14/765; C07K 14/77; C07K 14/79; C07K 14/81; C12N 9/36; G01N 33/543; G01N 33/68

(52) U.S. Cl. CPC C07K 14/465; C07K 14/765; C07K 14/77; C07K 14/79; C07K 14/8135; C12N 9/2462; G01N 33/54353; G01N 33/6854; A61K 38/00; G01N 2800/24

(57) ABSTRACT

The present disclosure provides egg peptide compositions and kits, and methods for diagnosis of egg allergy, methods for detecting the development of clinical tolerance to eggs, and methods for desensitizing an infant to egg allergens.

### REFERENCE TO SEQUENCE LISTING

This application includes a Sequence Listing filed electronically as a text file named 18969800502SEQ, created on May 6, 2022, with a size of 41 kilobytes. The Sequence Listing is incorporated herein by reference.

### FIELD

The present disclosure is directed, in part, to egg peptide compositions and kits, and methods for diagnosis of egg allergy, methods for detecting the development of clinical tolerance to eggs, and methods for desensitizing an infant to egg allergens.

### BACKGROUND

Food allergies are a common problem among adults and children, and symptoms may range from mild oral pruritus to potentially life-threatening anaphylactic shock. Food allergies are currently diagnosed by skin prick testing or oral provocation, and measurement of serum levels of specific IgE and, in some cases, other serum antibodies, such as IgG4. Although these tests indicate the likelihood of clinical reactivity, they do not distinguish the different phenotypes of food allergy or provide prognostic information. Current allergy tests also involve some level of risk to the patient. The relationship between current IgE testing and the actual clinical sensitivity of the patient is a weak one that is usually defined as a combination of reaction severity and the amount of allergen that provokes a reaction. Another limitation of current testing is the inability to determine whether or not pediatric patients will outgrow the allergy during childhood. In this case there is a positive but weak correlation between specific IgE level and the duration of clinical allergy.

More recently, it has been suggested that clinical reactivity to food allergens may correlate better with allergen-specific IgE on the epitope recognition level. It has been reported that patients with persistent or more severe allergic reactions recognize larger numbers of IgE epitopes, suggesting epitope mapping as an additional tool for allergy diagnosis and prediction. Spot membrane-based immunoassays have been used for epitope mapping. In this system, peptides are synthesized on the membrane and incubated with the patient's sera. The process requires a large number of peptides and is, therefore, error prone, time consuming, labor intensive, and expensive. Immunoassays in this format also require a large volume of patient serum.

The marked heterogeneity of clinical presentations for food allergy poses a challenge to successful management and treatment, and therefore precision medicine strategies are highly relevant to improve prevention, manage current cases and initiate new therapy in food allergy. Sensitive and specific biomarkers for determination of food allergy endotypes, risk of developing allergies, reaction severity, and prognosis with treatment are essential components in the path toward precision medicine (Sicherer et al., J. Allergy Clin. Immunol., 2015, 135, 357-67). In the past decade, there have been a number of studies evaluating the efficacy of oral immunotherapy (OIT) for the treatment of persistent food allergies (Wood et al., J. Allergy Clin. Immunol., 2016, 137, 1103-1110). Despite the improvement in clinical reactivity, OIT has been associated with significant adverse effects, with some experiencing anaphylaxis and 15% to 20% forced to discontinue therapy because of adverse reactions (Bird et al., J. Allergy Clin. Immunol. Pract., 2017; Keet Et al., J. Allergy Clin. Immunol., 2012, 129, 448-455; Longo et al., J. Allergy Clin. Immunol., 2008, 121, 343-7; Meglio et al., Pediatr. Allergy Immunol., 2008, 19, 412-419; Skripak et al., J. Allergy Clin. Immunol., 2008, 122, 1154-60; Staden et al., Allergy, 2007, 62, 1261-1269). In addition to adverse reactions, the response to OIT is typically not sustained once therapy is discontinued, i.e. patients are temporarily desensitized to allergens but do not achieve tolerance (Wood et al., J. Allergy Clin. Immunol., 2016, 137, 1103-1110; Burks et al., N. Engl. J. Med., 2012, 367, 233-243; Burks et al., J. Allergy Clin. Immunol., 2008, 121, 1344-1350; Burks, Arb. Paul Ehrlich Inst. Bundesinstitut Impfstoffe Biomed Arzneim Langen Hess, 2013, 97, 122-123; Gorelik et al., J. Allergy Clin. Immunol., 2015, 135, 1283-1292; and Keet et al., J. Allergy Clin. Immunol., 2013, 132, 737-739). However, it is clear that progress is being made and new food allergy therapies are close to FDA approval. These therapeutic approaches will benefit from a diagnostic and prognostic test which will help patients and their doctors understand the severity of the disease upon entry into therapy, monitor a patient while on therapy to assess progress or onset of an adverse reaction before it occurs, and track patient status once treatment is discontinued.

In hen's (Gallus gallus) egg allergy, ovomucoid (OVM) has been identified as the major allergenic protein (Cooke et al., J. Immunol., 1997, 159, 2026-2032; Bemhisel-Broadbent et al., J. Allergy Clin. Immunol., 1994, 93, 1047-1059; Honma et al., Arerugi, 1991, 40, 1167-1175; and Matsuda et al., J. Agric. Food Chem., 1983, 31, 942-946), with OVM-depleted egg-white having diminished effect during food challenges and on serological IgE levels (Urisu et al., J. Allergy Clin. Immunol., 1997, 100, 171-176; and Mine et al., J. Science Food Agric., 2001, 81, 1540-1546). Egg allergic children that generate a diverse OVM epitope-specific IgE repertoire present a more severe and persistent disease phenotype (Jarvinen et al., Allergy, 2007, 62, 758-765; Holen et al., Clin. Exp. Allergy, 2001, 31, 952-964; Yamada et al., Allergy, 2000, 55, 565-569; and Mine et al., J. Agric. Food Chem., 2002, 50, 2679-2683). Three other proteins in egg-white have been determined to be allergenic in a subset of patients: ovalbumin (OVA), ovotransferrin (OVT), and lysozyme (LYS) (Mine et al., J. Agric. Food Chem., 2002, 50, 2679-2683; Mine et al., J. Agric. Food Chem., 2008, 56, 4874-4900; Elsayed et al., Scand. J. Immunol., 1988, 27, 587-591; Johnsen et al., Mol. Immunol., 1990, 27, 821-827; Vance et al., Clin. Exp. Allergy, 2004, 34, 1542-1549; Walsh et al., Allergol Immunopathol (Madr), 2005, 33, 183-191; Darsley et al., EMBO J., 1985, 4, 383-392; Weber et al., Mol. Nutr. Food Res., 2009, 53, 1469-1477; Jimenez-Saiz et al., Mol. Nutr. Food Res., 2014, 58, 614-624; and Tong et al., Food Chemistry, 2012, 131, 603-610). Egg yolk is generally considered less allergenic, with IgE in adults mostly associated with bird-egg syndrome—a sensitization to airborne avian antigens (e.g., feathers, droppings) (de Maat-Bleeker et al., Ann. Allergy, 1985, 54, 245-248; Hemmer et al., Allergo J. Int., 2016, 25, 68-75; Mandallaz et al., Int. Arch Allergy Appl. Immunol., 1988, 87, 143-150; Szepfalusi et al., J. Allergy Clin. Immunol., 1994, 93, 932-942; and Bausela et al., Allergy, 1991, 46, 614-618). Nonetheless, several immunogenic proteins in yolk have been related to egg allergy: serum albumin (sALB), vitellogenin-1 precursor (YGP42) and vitellogenin-II fragment (YGP40) (Chruszcz et al., Biochim Biophys Acta, 2013, 1830, 5375-5381; Quirce et al., Allergy, 2001, 56, 754-762; Amo et al., J. Agric. Food Chem., 2010, 58, 7453-7457; Dhanapala et al., Mol. Immunol., 2015, 66, 375-383; Walsh et al., Int'l Arch. Allergy Immunol., 1988, 87, 81-86; Brossard et al., Ped. Allergy Immunol., 2019, 30, 225-233; and Sogawa et al., Int'l Arch Allergy Immunol., 2018, 176, 189-197). Aside from a few studies in OVA (Benede et al., J. Agric. Food Chem., 2014, 62, 152-158; and Mine et al., Protein Eng., 2003, 16, 747-752) and LYS (Jimenez-Saiz et al., Mol. Nutr. Food Res., 2014, 58, 614-624), much less is known about epitope mapping in minor allergens and the contribution of those IgE-binding epitopes to the pathogenesis of egg allergy (Matsuo et al., Allergol Int., 2015, 64, 332-343).

### SUMMARY

The present disclosure provides compositions comprising up to about 65 egg peptides from 1 to 7 egg proteins, wherein the egg proteins are selected from ovomucoid, ovalbumin, ovotransferrin, lysozyme, yolk serum albumin, YGP40, and YBG42; wherein the ovomucoid peptides are selected from: OVM-001 (AEVDCSRFPNATDKE; SEQ ID NO: 1), OVM-003 (RFPNATDKEGKDVLV; SEQ ID NO: 2), OVM-004 (NATDKEGKDVLVCNK; SEQ ID NO: 3), OVM-005 (DKEGKDVLVCNKDLR; SEQ ID NO: 4), OVM-007 (VLVCNKDLRPICGTD; SEQ ID NO: 5), OVM-008 (CNKDLRPICGTDGVT; SEQ ID NO: 6), OVM-009 (DLRPICGTDGVTYTN; SEQ ID NO: 7), OVM-010 (PICGTDGVTYTNDCL; SEQ ID NO: 8), OVM-013 (YTNDCLLCAYSIEFG; SEQ ID NO: 9), OVM-014 (DCLLCAYSIEFGTNI; SEQ ID NO: 10), OVM-015 (LCAYSIEFGTNISKE; SEQ ID NO: 11), OVM-018 (TNISKEHDGECKETV; SEQ ID NO: 12), OVM-021 (ECKETVPMNCSSYAN; SEQ ID NO: 13), OVM-023 (PMNCSSYANTTSEDG; SEQ ID NO: 14), OVM-025 (YANTTSEDGKVMVLC; SEQ ID NO: 15), OVM-027 (EDGKVMVLCNRAFNP; SEQ ID NO: 16), OVM-028 (KVMVLCNRAFNPVCG; SEQ ID NO: 17), OVM-031 (FNPVCGTDGVTYDNE; SEQ ID NO: 18), OVM-032 (VCGTDGVTYDNECLL; SEQ ID NO: 19), OVM-033 (TDGVTYDNECLLCAH; SEQ ID NO: 20), OVM-036 (CLLCAHKVEQGASVD; SEQ ID NO: 21), OVM-038 (KVEQGASVDKRHDGG; SEQ ID NO: 22), OVM-040 (SVDKRHDGGCRKELA; SEQ ID NO: 23), OVM-041 (KRHDGGCRKELAAVS; SEQ ID NO: 24), OVM-042 (DGGCRKELAAVSVDC; SEQ ID NO: 25), OVM-044 (ELAAVSVDCSEYPKP; SEQ ID NO: 26), OVM-046 (VDCSEYPKPDCTAED; SEQ ID NO: 27), OVM-048 (PKPDCTAEDRPLCGS; SEQ ID NO: 28), OVM-050 (AEDRPLCGSDNKTYG; SEQ ID NO: 29), OVM-052 (CGSDNKTYGNKCNFC; SEQ ID NO: 30), OVM-054 (TYGNKCNFCNAVVES; SEQ ID NO: 31), OVM-055 (NKCNFCNAVVESNGT; SEQ ID NO: 32), OVM-056 (NFCNAVVESNGTLTL; SEQ ID NO: 33), and/or OVM-058 (VESNGTLTLSHFGKC; SEQ ID NO: 34); wherein the ovalbumin peptides are selected from: OVA-001 (MGSIGAASMEFCFDV; SEQ ID NO: 35), OVA-003 (ASMEFCFDVFKELKV; SEQ ID NO: 36), OVA-006 (FKELKVHHANENIFY; SEQ ID NO: 37), OVA-021 (DKLPGFGDSIEAQCG; SEQ ID NO: 38), OVA-028 (SSLRDILNQITKPND; SEQ ID NO: 39), OVA-040 (YLQCVKELYRGGLEP; SEQ ID NO: 40), OVA-053 (IIRNVLQPSSVDSQT; SEQ ID NO: 41), OVA-057 (SQTAXVLVNAIVFKG; SEQ ID NO: 42), OVA-059 (LVNAIVFKGLWEKAF; SEQ ID NO: 43), OVA-064 (KDEDTQAMPFRVTEQ; SEQ ID NO: 44), OVA-068 (TEQESKPVQMMYQIG; SEQ ID NO: 45), OVA-071 (MMYQIGLFRVASMAS; SEQ ID NO: 46), OVA-075 (MASEKMKILELPFAS; SEQ ID NO: 47), OVA-085 (LEQLESIINFEKLTE; SEQ ID NO: 48), OVA-090 (WTSSNVMEERKIKVY; SEQ ID NO: 49), OVA-095 (LPRMKMEEKYNLTSV; SEQ ID NO: 50), OVA-108 (LKISQAVHAAHAEIN; SEQ ID NO: 51), OVA-113 (EAGREVVGSAEAGVD; SEQ ID NO: 52), and/or OVA-120 (EFRADHPFLFCIKHI; SEQ ID NO: 53); wherein the lysozyme peptides are selected from: LYS-1 (AAAMKRHGLDNYRGY; SEQ ID NO: 54), LYS-2 (GYSLGNWVCAAKFES; SEQ ID NO: 55). LYS-3 (RNTDGSTDYGILQTN; SEQ ID NO: 56), LYS-4 (TDYGILQINSRWWCN; SEQ ID NO: 57), LYS-5 (WCNDGRTPGSRNLCN; SEQ ID NO: 58), and/or LYS-6 (WVAWRNRCKGTDVQA; SEQ ID NO: 59); wherein the ovotransferrin peptides are selected from: OVT-1 (SSPEEKKCNNLRDLT; SEQ ID NO: 60), OVT-2 (LRDLTQQERISLTCV; SEQ ID NO: 61), OVT-3 (TIEQKLCRQCKGDPK; SEQ ID NO: 62), OVT-4 (DQLTPSPRENRIQWC; SEQ ID NO: 63), and/or OVT-5 (VMDYRECNLAEVPTH; SEQ ID NO: 64); wherein the serum albumin peptides are selected from: sALB-1 (YQRPASDVICQEYQD SEQ ID NO: 65 or LRDSYGAMADCCSKA SEQ ID NO: 66), sALB-2 (LEKCCKTDNPAECYA; SEQ ID NO: 67), sALB-3 (KQETTPINDNVSQCC; SEQ ID NO: 68), sALB-4 (GVDTKYVPPPFNPDM; SEQ ID NO: 69), and/or sALB-5 (MVDKCCKQSDINTCFGE; SEQ ID NO: 70); wherein the YGP40 peptides are selected from: YGP40-1 (NYSMPANCYHILVQD; SEQ ID NO: 71), YGP40-2 (VQDCSSELKFLVMMK; SEQ ID NO: 72), YGP40-3 (CAKGCSATKTTPVTV; SEQ ID NO: 73), YGP40-4 (CSATKTTPVTVGFHC; SEQ ID NO: 74), and/or YGP40-5 (FHCLPADSANSLTDK; SEQ ID NO: 75); and wherein the YBG42 peptides are selected from: YGP42-1 (VDHQSLSREVHINTS; SEQ ID NO: 76), YGP42-2 (GVCGNNDREKHNELL; SEQ ID NO: 77), and/or YGP42-3 (SSDKKSASEDVVESV; SEQ ID NO: 78); wherein the amino acid sequence of any one or more of the egg peptides can have one to four conservative amino acid substitutions therein.

The present disclosure also provides kits comprising: a solid support coupled to a plurality up to about 65 egg peptides from 1 to 7 egg proteins, wherein the egg proteins are selected from ovomucoid, ovalbumin, ovotransferrin, lysozyme, yolk serum albumin, YGP40, and YBG42; wherein the ovomucoid peptides are selected from: OVM-001 (AEVDCSRFPNATDKE; SEQ ID NO: 1), OVM-003 (RFPNATDKEGKDVLV; SEQ ID NO: 2), OVM-004 (NATDKEGKDVLVCNK; SEQ ID NO: 3), OVM-005 (DKEGKDVLVCNKDLR; SEQ ID NO: 4), OVM-007 (VLVCNKDLRPICGTD; SEQ ID NO: 5), OVM-008 (CNKDLRPICGTDGVT; SEQ ID NO: 6), OVM-009 (DLRPICGTDGVTYTN; SEQ ID NO: 7), OVM-010 (PICGTDGVTYTNDCL; SEQ ID NO: 8), OVM-013 (YTNDCLLCAYSIEFG; SEQ ID NO: 9), OVM-014 (DCLLCAYSIEFGTNI; SEQ ID NO: 10), OVM-015 (LCAYSIEFGTNISKE; SEQ ID NO: 11), OVM-018 (TNISKEHDGECKETV; SEQ ID NO: 12), OVM-021 (ECKETVPMNCSSYAN; SEQ ID NO: 13), OVM-023 (PMNCSSYANTTSEDG; SEQ ID NO: 14), OVM-025 (YANTTSEDGKVMVLC; SEQ ID NO: 15), OVM-027 (EDGKVMVLCNRAFNP; SEQ ID NO: 16), OVM-028 (KVMVLCNRAFNPVCG; SEQ ID NO: 17), OVM-031 (FNPVCGTDGVTYDNE; SEQ ID NO: 18), OVM-032 (VCGTDGVTYDNECLL; SEQ ID NO: 19), OVM-033 (TDGVTYDNECLLCAH; SEQ ID NO: 20), OVM-036 (CLLCAHKVEQGASVD; SEQ ID NO: 21), OVM-038 (KVEQGASVDKRHDGG; SEQ ID NO: 22), OVM-040 (SVDKRHDGGCRKELA; SEQ ID NO: 23), OVM-041 (KRHDGGCRKELAAVS; SEQ ID NO: 24), OVM-042 (DGGCRKELAAVSVDC; SEQ ID NO: 25), OVM-044 (ELAAVSVDCSEYPKP; SEQ ID NO: 26), OVM-046 (VDCSEYPKPDCTAED; SEQ ID NO: 27), OVM-048 (PKPDCTAEDRPLCGS; SEQ ID NO: 28), OVM-050 (AEDRPLCGSDNKTYG; SEQ ID NO: 29), OVM-052 (CGSDNKTYGNKCNFC; SEQ ID NO: 30), OVM-054 (TYGNKCNFCNAVVES; SEQ ID NO: 31), OVM-055 (NKCNFCNAVVESNGT; SEQ ID NO: 32), OVM-056 (NFCNAVVESNGTLTL; SEQ ID NO: 33), and/or OVM-058 (VESNGTLTLSHFGKC; SEQ ID NO: 34); wherein the ovalbumin peptides are selected from: OVA-001 (MGSIGAASMEFCFDV; SEQ ID NO: 35), OVA-003 (ASMEFCFDVFKELKV; SEQ ID NO: 36), OVA-006 (FKELKVHHANENIFY; SEQ ID NO: 37), OVA-021 (DKLPGFGDSIEAQCG; SEQ ID NO: 38), OVA-028 (SSLRDILNQITKPND; SEQ ID NO: 39), OVA-040 (YLQCVKELYRGGLEP; SEQ ID NO: 40), OVA-053 (IIRNVLQPSSVDSQT; SEQ ID NO: 41), OVA-057 (SQTAXVLVNAIVFKG; SEQ ID NO: 42), OVA-059 (LVNAIVFKGLWEKAF; SEQ ID NO: 43), OVA-064 (KDEDTQAMPFRVTEQ; SEQ ID NO: 44), OVA-068 (TEQESKPVQMMYQIG; SEQ ID NO: 45), OVA-071 (MMYQIGLFRVASMAS; SEQ ID NO: 46), OVA-075 (MASEKMKILELPFAS; SEQ ID NO: 47), OVA-085 (LEQLESIINFEKLTE; SEQ ID NO: 48), OVA-090 (WTSSNVMEERKIKVY; SEQ ID NO: 49), OVA-095 (LPRMKMEEKYNLTSV; SEQ ID NO: 50), OVA-108 (LKISQAVHAAHAEIN; SEQ ID NO: 51), OVA-113 (EAGREVVGSAEAGVD; SEQ ID NO: 52), and/or OVA-120 (EFRADHPFLFCIKHI; SEQ ID NO: 53); wherein the lysozyme peptides are selected from: LYS-1 (AAAMKRHGLDNYRGY; SEQ ID NO: 54), LYS-2 (GYSLGNWVCAAKFES; SEQ ID NO: 55), LYS-3 (RNTDGSTDYGILQIN; SEQ ID NO: 56), LYS-4 (TDYGILQINSRWWCN; SEQ ID NO: 57), LYS-5 (WCNDGRTPGSRNLCN; SEQ ID NO: 58), and/or LYS-6 (WVAWRNRCKGTDVQA; SEQ ID NO: 59); wherein the ovotransferrin peptides are selected from: OVT-1 (SSPEEKKCNNLRDLT; SEQ ID NO: 60), OVT-2 (LRDLTQQERISLTCV; SEQ ID NO: 61), OVT-3 (TIEQKLCRQCKGDPK; SEQ ID NO: 62), OVT-4 (DQLTPSPRENRIQWC; SEQ ID NO: 63), and/or OVT-5 (VMDYRECNLAEVPTH; SEQ ID NO: 64); wherein the serum albumin peptides are selected from: sALB-1 (YQRPASDVICQEYQD SEQ ID NO: 65 or LRDSYGAMADCCSKA SEQ ID NO: 66), sALB-2 (LEKCCKTDNPAECYA; SEQ ID NO: 67), sALB-3 (KQETTPINDNVSQCC; SEQ ID NO: 68), sALB-4 (GVDTKYVPPPFNPDM; SEQ ID NO: 69), and/or sALB-5 (MVDKCCKQSDINTCFGE; SEQ ID NO: 70); wherein the YGP40 peptides are selected from: YGP40-1 (NYSMPANCYHILVQD; SEQ ID NO: 71), YGP40-2 (VQDCSSELKFLVMMK; SEQ ID NO: 72), YGP40-3 (CAKGCSATKTTPVTV; SEQ ID NO: 73), YGP40-4 (CSATKTTPVTVGFHC; SEQ ID NO: 74), and/or YGP40-5 (FHCLPADSANSLTDK; SEQ ID NO: 75); and wherein the YBG42 peptides are selected from: YGP42-1 (VDHQSLSREVHINTS; SEQ ID NO: 76), YGP42-2 (GVCGNNDREKHNELL; SEQ ID NO: 77), and/or YGP42-3 (SSDKKSASEDVVESV; SEQ ID NO: 78); wherein the amino acid sequence of any one or more of the egg peptides can have one to four conservative amino acid substitutions therein; and an allergy associated immunoglobulin (AAI)-specific labeling reagent.

The present disclosure also provides methods for diagnosing an egg allergy in a subject comprising: contacting a plurality of egg peptides coupled to a solid support with a biological sample obtained from the subject under conditions sufficient to permit binding of one or more allergy associated immunoglobulins (AAIs) in the biological sample to egg peptides to form AAI-peptide-solid support complexes, wherein the plurality of egg peptides comprises up to about 65 egg peptides from 1 to 7 egg proteins, wherein the egg proteins are selected from ovomucoid, ovalbumin, ovotransferrin, lysozyme, yolk serum albumin, YGP40, and YBG42; wherein the ovomucoid peptides are selected from: OVM-001 (AEVDCSRFPNATDKE; SEQ ID NO: 1), OVM-003 (RFPNATDKEGKDVLV; SEQ ID NO: 2), OVM-004 (NATDKEGKDVLVCNK; SEQ ID NO: 3), OVM-005 (DKEGKDVLVCNKDLR; SEQ ID NO: 4), OVM-007 (VLVCNKDLRPICGTD; SEQ ID NO: 5), OVM-008 (CNKDLRPICGTDGVT; SEQ ID NO: 6), OVM-009 (DLRPICGTDGVTYTN; SEQ ID NO: 7), OVM-010 (PICGTDGVTYTNDCL; SEQ ID NO: 8), OVM-013 (YTNDCLLCAYSIEFG; SEQ ID NO: 9), OVM-014 (DCLLCAYSIEFGTNI; SEQ ID NO: 10), OVM-015 (LCAYSIEFGTNISKE; SEQ ID NO: 11), OVM-018 (TNISKEHDGECKETV; SEQ ID NO: 12), OVM-021 (ECKETVPMNCSSYAN; SEQ ID NO: 13), OVM-023 (PMNCSSYANTTSEDG; SEQ ID NO: 14), OVM-025 (YANTTSEDGKVMVLC; SEQ ID NO: 15), OVM-027 (EDGKVMVLCNRAFNP; SEQ ID NO: 16), OVM-028 (KVMVLCNRAFNPVCG; SEQ ID NO: 17), OVM-031 (FNPVCGTDGVTYDNE; SEQ ID NO: 18), OVM-032 (VCGTDGVTYDNECLL; SEQ ID NO: 19), OVM-033 (TDGVTYDNECLLCAH; SEQ ID NO: 20), OVM-036 (CLLCAHKVEQGASVD; SEQ ID NO: 21), OVM-038 (KVEQGASVDKRHDGG; SEQ ID NO: 22), OVM-040 (SVDKRHDGGCRKELA; SEQ ID NO: 23), OVM-041 (KRHDGGCRKELAAVS; SEQ ID NO: 24), OVM-042 (DGGCRKELAAVSVDC; SEQ ID NO: 25), OVM-044 (ELAAVSVDCSEYPKP; SEQ ID NO: 26), OVM-046 (VDCSEYPKPDCTAED; SEQ ID NO: 27), OVM-048 (PKPDCTAEDRPLCGS; SEQ ID NO: 28), OVM-050 (AEDRPLCGSDNKTYG; SEQ ID NO: 29), OVM-052 (CGSDNKTYGNKCNFC; SEQ ID NO: 30), OVM-054 (TYGNKCNFCNAVVES; SEQ ID NO: 31), OVM-055 (NKCNFCNAVVESNGT; SEQ ID NO: 32), OVM-056 (NFCNAVVESNGTLTL; SEQ ID NO: 33), and/or OVM-058 (VESNGTLTLSHFGKC; SEQ ID NO: 34); wherein the ovalbumin peptides are selected from: OVA-001 (MGSIGAASMEFCFDV; SEQ ID NO: 35), OVA-003 (ASMEFCFDVFKELKV; SEQ ID NO: 36), OVA-006 (FKELKVHHANENIFY; SEQ ID NO: 37), OVA-021 (DKLPGFGDSIEAQCG; SEQ ID NO: 38), OVA-028 (SSLRDILNQITKPND; SEQ ID NO: 39), OVA-040 (YLQCVKELYRGGLEP; SEQ ID NO: 40), OVA-053 (IIRNVLQPSSVDSQT; SEQ ID NO: 41), OVA-057 (SQTAXVLVNAIVFKG; SEQ ID NO: 42), OVA-059 (LVNAIVFKGLWEKAF; SEQ ID NO: 43), OVA-064 (KDEDTQAMPFRVTEQ; SEQ ID NO: 44), OVA-068 (TEQESKPVQMMYQIG; SEQ ID NO: 45), OVA-071 (MMYQIGLFRVASMAS; SEQ ID NO: 46), OVA-075 (MASEKMKILELPFAS; SEQ ID NO: 47), OVA-085 (LEQLESIINFEKLTE; SEQ ID NO: 48), OVA-090 (WTSSNVMEERKIKVY; SEQ ID NO: 49), OVA-095 (LPRMKMEEKYNLTSV; SEQ ID NO: 50), OVA-108 (LKISQAVHAAHAEIN; SEQ ID NO: 51), OVA-113 (EAGREVVGSAEAGVD; SEQ ID NO: 52), and/or OVA-120 (EFRADHPFLFCIKHI; SEQ ID NO: 53); wherein the lysozyme peptides are selected from: LYS-1 (AAAMKRHGLDNYRGY; SEQ ID NO: 54), LYS-2 (GYSLGNWVCAAKFES; SEQ ID NO: 55), LYS-3 (RNTDGSTDYGILQIN; SEQ ID NO: 56), LYS-4 (TDYGILQINSRWWCN; SEQ ID NO: 57), LYS-5 (WCNDGRTPGSRNLCN; SEQ ID NO: 58), and/or LYS-6 (WVAWRNRCKGTDVQA; SEQ ID NO: 59); wherein the ovotransferrin peptides are selected from: OVT-1 (SSPEEKKCNNLRDLT; SEQ ID NO: 60), OVT-2 (LRDLTQQERISLTCV; SEQ ID NO: 61), OVT-3 (TIEQKLCRQCKGDPK; SEQ ID NO: 62), OVT-4 (DQLTPSPRENRIQWC; SEQ ID NO: 63), and/or OVT-5 (VMDYRECNLAEVPTH; SEQ ID NO: 64); wherein the serum albumin peptides are selected from: sALB-1 (YQRPASDVICQEYQD SEQ ID NO: 65 or LRDSYGAMADCCSKA SEQ ID NO: 66), sALB-2 (LEKCCKTDNPAECYA; SEQ ID NO: 67), sALB-3 (KQETTPINDNVSQCC; SEQ ID NO: 68), sALB-4 (GVDTKYVPPPFNPDM; SEQ ID NO: 69), and/or sALB-5 (MVDKCCKQSDINTCFGE; SEQ ID NO: 70); wherein the YGP40 peptides are selected from: YGP40-1 (NYSMPANCYHILVQD; SEQ ID NO: 71), YGP40-2 (VQDCSSELKFLVMMK; SEQ ID NO: 72), YGP40-3 (CAKGCSATKTTPVTV; SEQ ID NO: 73), YGP40-4 (CSATKTTPVTVGFHC; SEQ ID NO: 74), and/or YGP40-5 (FHCLPADSANSLTDK; SEQ ID NO: 75); and wherein the YBG42 peptides are selected from: YGP42-1 (VDHQSLSREVHINTS; SEQ ID NO: 76), YGP42-2 (GVCGNNDREKHNELL; SEQ ID NO: 77), and/or YGP42-3 (SSDKKSASEDVVESV; SEQ ID NO: 78); wherein the amino acid sequence of any one or more of the egg peptides can have one to four conservative amino acid substitutions therein; contacting the AAI-peptide-solid support complexes with an AAI-specific labeling reagent to form labeling reagent-AAI-peptide-solid support complexes; and measuring the binding of the AAI-specific labeling reagent to each AAI-peptide-solid support complex; wherein when the combined binding for each egg peptide in the AAI-peptide solid support complex to the AAI-specific labeling reagent is greater than a threshold value, the subject is allergic to eggs, and when the combined binding for each egg peptide in the AAI-peptide solid support complex to the AAI-specific labeling reagent is equal to or less than the threshold value, the subject is not allergic to eggs.

The present disclosure also provides methods for detecting development of clinical tolerance to eggs in a subject that is allergic to eggs comprising: contacting a plurality of egg peptides coupled to a solid support with a biological sample obtained from the subject under conditions sufficient to permit binding of one or more allergy associated immunoglobulins (AAIs) in the biological sample to egg peptides to form AAI-peptide-solid support complexes, wherein the plurality of egg peptides comprises up to about 65 egg peptides from 1 to 7 egg proteins, wherein the egg proteins are selected from ovomucoid, ovalbumin, ovotransferrin, lysozyme, yolk serum albumin, YGP40, and YBG42; wherein the ovomucoid peptides are selected from: OVM-001 (AEVDCSRFPNATDKE; SEQ ID NO: 1), OVM-003 (RFPNATDKEGKDVLV; SEQ ID NO: 2), OVM-004 (NATDKEGKDVLVCNK; SEQ ID NO: 3), OVM-005 (DKEGKDVLVCNKDLR; SEQ ID NO: 4), OVM-007 (VLVCNKDLRPICGTD; SEQ ID NO: 5), OVM-008 (CNKDLRPICGTDGVT; SEQ ID NO: 6), OVM-009 (DLRPICGTDGVTYTN; SEQ ID NO: 7), OVM-010 (PICGTDGVTYTNDCL; SEQ ID NO: 8), OVM-013 (YTNDCLLCAYSIEFG; SEQ ID NO: 9), OVM-014 (DCLLCAYSIEFGTNI; SEQ ID NO: 10), OVM-015 (LCAYSIEFGTNISKE; SEQ ID NO: 11), OVM-018 (TNISKEHDGECKETV; SEQ ID NO: 12), OVM-021 (ECKETVPMNCSSYAN; SEQ ID NO: 13), OVM-023 (PMNCSSYANTTSEDG; SEQ ID NO: 14), OVM-025 (YANTTSEDGKVMVLC; SEQ ID NO: 15), OVM-027 (EDGKVMVLCNRAFNP; SEQ ID NO: 16), OVM-028 (KVMVLCNRAFNPVCG; SEQ ID NO: 17), OVM-031 (FNPVCGTDGVTYDNE; SEQ ID NO: 18), OVM-032 (VCGTDGVTYDNECLL; SEQ ID NO: 19), OVM-033 (TDGVTYDNECLLCAH; SEQ ID NO: 20), OVM-036 (CLLCAHKVEQGASVD; SEQ ID NO: 21), OVM-038 (KVEQGASVDKRHDGG; SEQ ID NO: 22), OVM-040 (SVDKRHDGGCRKELA; SEQ ID NO: 23), OVM-041 (KRHDGGCRKELAAVS; SEQ ID NO: 24), OVM-042 (DGGCRKELAAVSVDC; SEQ ID NO: 25), OVM-044 (ELAAVSVDCSEYPKP; SEQ ID NO: 26), OVM-046 (VDCSEYPKPDCTAED; SEQ ID NO: 27), OVM-048 (PKPDCTAEDRPLCGS; SEQ ID NO: 28), OVM-050 (AEDRPLCGSDNKTYG; SEQ ID NO: 29), OVM-052 (CGSDNKTYGNKCNFC; SEQ ID NO: 30), OVM-054 (TYGNKCNFCNAVVES; SEQ ID NO: 31), OVM-055 (NKCNFCNAVVESNGT; SEQ ID NO: 32), OVM-056 (NFCNAVVESNGTLTL; SEQ ID NO: 33), and/or OVM-058 (VESNGTLTLSHFGKC; SEQ ID NO: 34); wherein the ovalbumin peptides are selected from: OVA-001 (MGSIGAASMEFCFDV; SEQ ID NO: 35), OVA-003 (ASMEFCFDVFKELKV; SEQ ID NO: 36), OVA-006 (FKELKVHHANENIFY; SEQ ID NO: 37), OVA-021 (DKLPGFGDSIEAQCG; SEQ ID NO: 38), OVA-028 (SSLRDILNQITKPND; SEQ ID NO: 39), OVA-040 (YLQCVKELYRGGLEP; SEQ ID NO: 40), OVA-053 (IIRNVLQPSSVDSQT; SEQ ID NO: 41), OVA-057 (SQTAXVLVNAIVFKG; SEQ ID NO: 42), OVA-059 (LVNAIVFKGLWEKAF; SEQ ID NO: 43), OVA-064 (KDEDTQAMPFRVTEQ; SEQ ID NO: 44), OVA-068 (TEQESKPVQMMYQIG; SEQ ID NO: 45), OVA-071 (MMYQIGLFRVASMAS; SEQ ID NO: 46), OVA-075 (MASEKMKILELPFAS; SEQ ID NO: 47), OVA-085 (LEQLESIINFEKLTE; SEQ ID NO: 48), OVA-090 (WTSSNVMEERKIKVY; SEQ ID NO: 49), OVA-095 (LPRMKMEEKYNLTSV; SEQ ID NO: 50), OVA-108 (LKISQAVHAAHAEIN; SEQ ID NO: 51), OVA-113 (EAGREVVGSAEAGVD; SEQ ID NO: 52), and/or OVA-120 (EFRADHPFLFCIKHI; SEQ ID NO: 53); wherein the lysozyme peptides are selected from: LYS-1 (AAAMKRHGLDNYRGY; SEQ ID NO: 54), LYS-2 (GYSLGNWVCAAKFES; SEQ ID NO: 55), LYS-3 (RNTDGSTDYGILQIN; SEQ ID NO: 56), LYS-4 (TDYGILQINSRWWCN; SEQ ID NO: 57), LYS-5 (WCNDGRTPGSRNLCN; SEQ ID NO: 58), and/or LYS-6 (WVAWRNRCKGTDVQA; SEQ ID NO: 59); wherein the ovotransferrin peptides are selected from: OVT-1 (SSPEEKKCNNLRDLT; SEQ ID NO: 60), OVT-2 (LRDLTQQERISLTCV; SEQ ID NO: 61), OVT-3 (TIEQKLCRQCKGDPK; SEQ ID NO: 62), OVT-4 (DQLTPSPRENRIQWC; SEQ ID NO: 63), and/or OVT-5 (VMDYRECNLAEVPTH; SEQ ID NO: 64); wherein the serum albumin peptides are selected from: sALB-1 (YQRPASDVICQEYQD SEQ ID NO: 65 or LRDSYGAMADCCSKA SEQ ID NO: 66), sALB-2 (LEKCCKTDNPAECYA; SEQ ID NO: 67), sALB-3 (KQETTPINDNVSQCC; SEQ ID NO: 68), sALB-4 (GVDTKYVPPPFNPDM; SEQ ID NO: 69), and/or sALB-5 (MVDKCCKQSDINTCFGE; SEQ ID NO: 70); wherein the YGP40 peptides are selected from: YGP40-1 (NYSMPANCYHILVQD; SEQ ID NO: 71), YGP40-2 (VQDCSSELKFLVMMK; SEQ ID NO: 72), YGP40-3 (CAKGCSATKTTPVTV; SEQ ID NO: 73), YGP40-4 (CSATKTTPVTVGFHC; SEQ ID NO: 74), and/or YGP40-5 (FHCLPADSANSLTDK; SEQ ID NO: 75); and wherein the YBG42 peptides are selected from: YGP42-1 (VDHQSLSREVHINTS; SEQ ID NO: 76), YGP42-2 (GVCGNNDREKHNELL; SEQ ID NO: 77), and/or YGP42-3 (SSDKKSASEDVVESV; SEQ ID NO: 78); wherein the amino acid sequence of any one or more of the egg peptides can have one to four conservative amino acid substitutions therein; contacting the AAI-peptide-solid support complexes with an AAI-specific labeling reagent to form labeling reagent-AAI-peptide-solid support complexes; measuring the binding of the AAI-specific labeling reagent to each AAI-peptide-solid support complex; and comparing the binding of the AAI-specific labeling reagent to each AAI-peptide-solid support complex to the binding of the AAI-specific labeling reagent to each AAI-peptide-solid support complex from a biological sample previously obtained from the subject; wherein when the combined binding for each egg peptide in the AAI-peptide solid support complex to the AAI-specific labeling reagent is greater than or equal to the combined binding for each egg peptide in the AAI-peptide solid support complex to the AAI-specific labeling reagent for the previously obtained biological sample, the subject has not established clinical tolerance to eggs; and when the combined binding for each egg peptide in the AAI-peptide solid support complex to the AAI-specific labeling reagent is less than the combined binding for each egg peptide in the AAI-peptide solid support complex to the AAI-specific labeling reagent for the previously obtained biological sample, the subject has established clinical tolerance to eggs.

The present disclosure also provides methods of desensitizing an infant to egg allergens to induce tolerance or non-allergy to eggs comprising administering to the infant a plurality of egg peptides, wherein the plurality of egg peptides comprises up to about 65 egg peptides from 1 to 7 egg proteins, wherein the egg proteins are selected from ovomucoid, ovalbumin, ovotransferrin, lysozyme, yolk serum albumin, YGP40, and YBG42; wherein the ovomucoid peptides are selected from: OVM-001 (AEVDCSRFPNATDKE; SEQ ID NO: 1), OVM-003 (RFPNATDKEGKDVLV; SEQ ID NO: 2), OVM-004 (NATDKEGKDVLVCNK; SEQ ID NO: 3), OVM-005 (DKEGKDVLVCNKDLR; SEQ ID NO: 4), OVM-007 (VLVCNKDLRPICGTD; SEQ ID NO: 5), OVM-008 (CNKDLRPICGTDGVT; SEQ ID NO: 6), OVM-009 (DLRPICGTDGVTYTN; SEQ ID NO: 7), OVM-010 (PICGTDGVTYTNDCL; SEQ ID NO: 8), OVM-013 (YTNDCLLCAYSIEFG; SEQ ID NO: 9), OVM-014 (DCLLCAYSIEFGTNI; SEQ ID NO: 10), OVM-015 (LCAYSIEFGTNISKE; SEQ ID NO: 11), OVM-018 (TNISKEHDGECKETV; SEQ ID NO: 12), OVM-021 (ECKETVPMNCSSYAN; SEQ ID NO: 13), OVM-023 (PMNCSSYANTTSEDG; SEQ ID NO: 14), OVM-025 (YANTTSEDGKVMVLC; SEQ ID NO: 15), OVM-027 (EDGKVMVLCNRAFNP; SEQ ID NO: 16), OVM-028 (KVMVLCNRAFNPVCG; SEQ ID NO: 17), OVM-031 (FNPVCGTDGVTYDNE; SEQ ID NO: 18), OVM-032 (VCGTDGVTYDNECLL; SEQ ID NO: 19), OVM-033 (TDGVTYDNECLLCAH; SEQ ID NO: 20), OVM-036 (CLLCAHKVEQGASVD; SEQ ID NO: 21), OVM-038 (KVEQGASVDKRHDGG; SEQ ID NO: 22), OVM-040 (SVDKRHDGGCRKELA; SEQ ID NO: 23), OVM-041 (KRHDGGCRKELAAVS; SEQ ID NO: 24), OVM-042 (DGGCRKELAAVSVDC; SEQ ID NO: 25), OVM-044 (ELAAVSVDCSEYPKP; SEQ ID NO: 26), OVM-046 (VDCSEYPKPDCTAED; SEQ ID NO: 27), OVM-048 (PKPDCTAEDRPLCGS; SEQ ID NO: 28), OVM-050 (AEDRPLCGSDNKTYG; SEQ ID NO: 29), OVM-052 (CGSDNKTYGNKCNFC; SEQ ID NO: 30), OVM-054 (TYGNKCNFCNAVVES; SEQ ID NO: 31), OVM-055 (NKCNFCNAVVESNGT; SEQ ID NO: 32), OVM-056 (NFCNAVVESNGTLTL; SEQ ID NO: 33), and/or OVM-058 (VESNGTLTLSHFGKC; SEQ ID NO: 34); wherein the ovalbumin peptides are selected from: OVA-001 (MGSIGAASMEFCFDV; SEQ ID NO: 35), OVA-003 (ASMEFCFDVFKELKV; SEQ ID NO: 36), OVA-006 (FKELKVHHANENIFY; SEQ ID NO: 37), OVA-021 (DKLPGFGDSIEAQCG; SEQ ID NO: 38), OVA-028 (SSLRDILNQITKPND; SEQ ID NO: 39), OVA-040 (YLQCVKELYRGGLEP; SEQ ID NO: 40), OVA-053 (IIRNVLQPSSVDSQT; SEQ ID NO: 41), OVA-057 (SQTAXVLVNAIVFKG; SEQ ID NO: 42), OVA-059 (LVNAIVFKGLWEKAF; SEQ ID NO: 43), OVA-064 (KDEDTQAMPFRVTEQ; SEQ ID NO: 44), OVA-068 (TEQESKPVQMMYQIG; SEQ ID NO: 45), OVA-071 (MMYQIGLFRVASMAS; SEQ ID NO: 46), OVA-075 (MASEKMKILELPFAS; SEQ ID NO: 47), OVA-085 (LEQLESIINFEKLTE; SEQ ID NO: 48), OVA-090 (WTSSNVMEERKIKVY; SEQ ID NO: 49), OVA-095 (LPRMKMEEKYNLTSV; SEQ ID NO: 50), OVA-108 (LKISQAVHAAHAEIN; SEQ ID NO: 51), OVA-113 (EAGREVVGSAEAGVD; SEQ ID NO: 52), and/or OVA-120 (EFRADHPFLFCIKHI; SEQ ID NO: 53); wherein the lysozyme peptides are selected from: LYS-1 (AAAMKRHGLDNYRGY; SEQ ID NO: 54), LYS-2 (GYSLGNWVCAAKFES; SEQ ID NO: 55), LYS-3 (RNTDGSTDYGILQIN; SEQ ID NO: 56), LYS-4 (TDYGILQINSRWWCN; SEQ ID NO: 57), LYS-5 (WCNDGRTPGSRNLCN; SEQ ID NO: 58), and/or LYS-6 (WVAWRNRCKGTDVQA; SEQ ID NO: 59); wherein the ovotransferrin peptides are selected from: OVT-1 (SSPEEKKCNNLRDLT; SEQ ID NO: 60), OVT-2 (LRDLTQQERISLTCV; SEQ ID NO: 61), OVT-3 (TIEQKLCRQCKGDPK; SEQ ID NO: 62), OVT-4 (DQLTPSPRENRIQWC; SEQ ID NO: 63), and/or OVT-5 (VMDYRECNLAEVPTH; SEQ ID NO: 64); wherein the serum albumin peptides are selected from: sALB-1 (YQRPASDVICQEYQD SEQ ID NO: 65 or LRDSYGAMADCCSKA SEQ ID NO: 66), sALB-2 (LEKCCKTDNPAECYA; SEQ ID NO: 67), sALB-3 (KQETTPINDNVSQCC; SEQ ID NO: 68), sALB-4 (GVDTKYVPPPFNPDM; SEQ ID NO: 69), and/or sALB-5 (MVDKCCKQSDINTCFGE; SEQ ID NO: 70); wherein the YGP40 peptides are selected from: YGP40-1 (NYSMPANCYHILVQD; SEQ ID NO: 71), YGP40-2 (VQDCSSELKFLVMMK; SEQ ID NO: 72), YGP40-3 (CAKGCSATKTTPVTV; SEQ ID NO: 73), YGP40-4 (CSATKTTPVTVGFHC; SEQ ID NO: 74), and/or YGP40-5 (FHCLPADSANSLTDK; SEQ ID NO: 75); and wherein the YBG42 peptides are selected from: YGP42-1 (VDHQSLSREVHINTS; SEQ ID NO: 76), YGP42-2 (GVCGNNDREKHNELL; SEQ ID NO: 77), and/or YGP42-3 (SSDKKSASEDVVESV; SEQ ID NO: 78); wherein the amino acid sequence of any one or more of the egg peptides can have one to four conservative amino acid substitutions therein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A depicts IgE epitope mapping on OVM proteins by nMFI of 58 OVM 15-mer sequential peptides measured using BBEA in 38 egg allergic children; color shows average nMFI, peptides are arranged in the sequential order on the y-axis, and x-axis represents a proportion on patients with that peptide “present.”

FIG. 1B depicts IgE epitope mapping on OVA proteins by nMFI of 125 OVA 15-mer sequential peptides measured using BBEA in 38 egg allergic children; color shows average nMFI, peptides are arranged in the sequential order on the y-axis, and x-axis represents a proportion on patients with that peptide “present.”

FIG. 1C depicts an epitope score for OVM for each amino acid based on the average nMFI and proportion of patients with peptide “present”; color represents score percentile across all amino acids; dashed line for OVM separates three domains; squared shape is a glycosylation site; letters on a side indicate potential sites of enzymatic cleavage by pepsin (P), trypsin (T) and chymotrypsin (Ch).

FIG. 1D depicts epitope mapping to the conformational structure of the OVM (obtained by homology modeling); epitopes are presented in different colors and correspond to the amino acids from FIG. 1C, with scores above 50th percentile.

FIG. 1E depicts an epitope score for OVA for each amino acid based on the average nMFI and proportion of patients with peptide “present”; color represents score percentile across all amino acids; squared shape is a glycosylation site; letters on a side indicate potential sites of enzymatic cleavage by pepsin (P), trypsin (T) and chymotrypsin (Ch).

FIG. 1F depicts epitope mapping to the conformational structure of the OVA (PDB: 1OVA); epitopes are presented in different colors and correspond to the amino acids from FIG. 1E, with scores above 50th percentile.

FIG. 2A depicts IgE epitope mapping on OVM and OVA using BBEA and in silico tools; heatmap showing amino acid-level epitope mapping by BBEA (1st column), and epitope predictions using random forest (RF)-based ensemble (2nd column), epitope prediction by at least 6 tools (2nd column), and 12 individual algorithms.

FIG. 2B depicts IgE epitope mapping on OVM and OVA using BBEA and in silico tools; importance metric (mean decrease in Gini index) for all 12 algorithms; higher values mean higher importance.

FIG. 3A depicts egg epitope library; median and 3rd quartile of nMFI for all OVM and OVA peptides and 24 potential epitopes from OVT, LYS, sALB, YGP42, and YGP40 proteins, measured using BBEA in 38 egg allergic children.

FIG. 3B depicts egg epitope library; heatmap of row-scaled nMFIs for all peptides tested with BBEA; peptides are arranged by their sequential order within a protein; subjects are grouped by unsupervised hierarchical clustering with Euclidean distance and “average” agglomeration algorithm; the right cluster (highlighted with pink horizontal bar) shows less IgE binding to OVM epitopes.

FIG. 3C depicts egg epitope library; schematic and a number of epitopes selected from each egg protein for the final 65-plex library.

FIG. 4A depicts association of ses-IgE and ses-IgG4 with egg allergy; EMmean of the nMFI for each of the 65 IgE and IgG4 binding epitopes in egg allergic subjects, atopic and healthy children; stars in the “egg allergic” column represent significant difference between allergic and atopic subjects; in the “healthy” column—allergic vs healthy subjects; in the “atopic” column—atopic vs healthy subjects. ***FDR <0.05, *p-value <0.05.

FIG. 4B depicts association of ses-IgE and ses-IgG4 with egg allergy; Z-score of 17 ses-IgE and ses-IgG4, defined as FDR <0.05 for BER vs BET comparison, compared using age adjusted linear regression model.

FIG. 4C depicts association of ses-IgE and ses-IgG4 with egg allergy; EMmean of the nMFI for each of the 65 IgE and IgG4 binding epitopes in BER, BET, atopic and healthy children; colored bars represent significant difference from BER (red if higher and blue if lower than BER) based on FDR (red/dark blue) or p-values (pink/light blue); stars atop of the bars represent significant difference from healthy subjects, based on the p-value.

FIG. 4D depicts association of ses-IgE and ses-IgG4 with egg allergy; boxplot of IgE and IgG4 levels specific to the whole OVM or OVA allergen extract, comparing BER and BET groups.

FIG. 5A depicts a representative study schematic summarizing representative steps.

FIG. 5B depicts unscaled nMFI values of IgE to all OVM and OVA peptides tested using BBEA in 38 egg allergic subjects and a negative pool (NP).

FIG. 5C depicts a review of the OVM epitope mapping literature; OVM epitopes identified by groups using different technologies; bottom row represents epitopes mapped in the current study and correspond to FIG. 1C.

FIG. 5D depicts a review of the OVA epitope mapping literature; OVA epitopes identified by groups using different technologies; bottom row represents epitopes mapped in the current study and correspond to FIG. 1E.

FIG. 5E depicts an example of the OVM's epitope selection for the 65-plex egg epitope library; peptides selected based on the unsupervised clustering (manhattan distance and mcquitty agglomeration algorithm) of column-scaled nMFI values.

FIG. 5F depicts an example of the OVM's epitope selection for the 65-plex egg epitope library; peptides selected based on the unsupervised clustering (manhattan distance and mcquitty agglomeration algorithm) of extreme values of 1st and/or 2nd principal component (PC).

FIG. 6 depicts an association of ses-IgE with egg allergy; Z-score of 17 ses-IgE, defined as FDR <0.05 for BER vs BET comparison, for subjects with z-score <4; this plot corresponds to FIG. 4B (top), highlighting the differences for low z-score values.

### DESCRIPTION OF EMBODIMENTS

The terminology used herein is for the purpose of describing particular embodiments only and is not intended to be limiting.

Before describing several exemplary embodiments, it is to be understood that the embodiments is not limited to the details of construction or process steps set forth in the following description. The embodiments described herein are capable of modifications and of being practiced or being carried out in various ways.

Reference throughout the present disclosure to “some embodiments,” or derivations thereof, means that a particular feature, structure, material, or characteristic described in connection with the embodiment is included in at least one embodiment. Thus, the appearances of the phrases such as “in some embodiments,” in various places throughout the present disclosure is not necessarily referring to the same embodiment, but can generally be attributed to any other embodiment. Furthermore, the particular features, structures, materials, or characteristics may be combined in any suitable manner in one or more embodiments.

As used herein, the terms “allergy associated immunoglobulin” and “AAI” refer to immunoglobulins in sera that mediate hypersensitivity to egg allergens. These include one or more of IgE, IgD, IgA, IgM, and IgG (including IgG4).

As used herein, the terms “reactive”, “reactivity”, “recognize” and the like refer to the ability of an allergy associated immunoglobulin to bind to an allergenic epitope containing peptide. The level of reactivity indicates the concentration of AAI in the serum or plasma, with high reactivity associated with higher AAI concentrations and lower reactivity associated with lower AAI concentrations. The relative AAI concentration (i.e., the relative serum or plasma reactivity) is determined by the amount of signal detected in an assay. The level of reactivity of AAI to allergenic epitope containing peptides also indicates the intensity of the allergic response (i.e., higher reactivity is associated with a more intense allergic reaction).

As used herein, the term “clinical tolerance” refers to immunological tolerance to an egg allergen that is developed by an allergic subject as a result of exposure to the allergen (i.e., tolerance developed as a result of immunotherapy).

As used herein, the term “natural tolerance” refers to immunological tolerance to an egg allergen that is developed by an allergic subject as a biochemical process over time, either as a result of natural exposure to the allergen during a lifetime or in the absence of exposure.

The present disclosure provides egg peptide compositions. In some embodiments, the egg peptide composition comprises or consists of: at least about 65 egg peptides, at least about 60 egg peptides, at least about 55 egg peptides, at least about 50 egg peptides, at least about 45 egg peptides, at least about 40 egg peptides, at least about 35 egg peptides, at least about 30 egg peptides, at least about 25 egg peptides, at least about 20 egg peptides, at least about 15 egg peptides, at least about 10 egg peptides, at least about 9 egg peptides, or at least about 8 egg peptides. In some embodiments, the egg peptide composition comprises or consists of at least about 65 egg peptides. In some embodiments, the egg peptide composition comprises or consists of at least about 60 egg peptides. In some embodiments, the egg peptide composition comprises or consists of at least about 55 egg peptides. In some embodiments, the egg peptide composition comprises or consists of at least about 50 egg peptides. In some embodiments, the egg peptide composition comprises or consists of at least about 45 egg peptides. In some embodiments, the egg peptide composition comprises or consists of at least about 40 egg peptides. In some embodiments, the egg peptide composition comprises or consists of at least about 35 egg peptides. In some embodiments, the egg peptide composition comprises or consists of at least about 30 egg peptides. In some embodiments, the egg peptide composition comprises or consists of at least about 25 egg peptides. In some embodiments, the egg peptide composition comprises or consists of at least about 20 egg peptides. In some embodiments, the egg peptide composition comprises or consists of at least about 15 egg peptides. In some embodiments, the egg peptide composition comprises or consists of at least about 10 egg peptides. In some embodiments, the egg peptide composition comprises or consists of at least about 9 egg peptides. In some embodiments, the egg peptide composition comprises or consists of at least about 8 egg peptides.

In some embodiments, the egg peptide composition comprises or consists from about 50 to about 80 egg peptides, from about 55 to about 75 egg peptides, from about 60 to about 70 egg peptides, or about 65 egg peptides. In some embodiments, the egg peptide composition comprises or consists from about 50 to about 80 egg peptides. In some embodiments, the egg peptide composition comprises or consists from about 55 to about 75 egg peptides. In some embodiments, the egg peptide composition comprises or consists from about 60 to about 70 egg peptides. In some embodiments, the egg peptide composition comprises or consists about 65 egg peptides.

In some embodiments, the egg peptide composition comprises or consists from about 18 to about 30 egg peptides, from about 20 to about 28 egg peptides, from about 22 to about 26 egg peptides, or about 24 egg peptides. In some embodiments, the egg peptide composition comprises or consists from about 18 to about 30 egg peptides. In some embodiments, the egg peptide composition comprises or consists from about 20 to about 28 egg peptides. In some embodiments, the egg peptide composition comprises or consists from about 22 to about 26 egg peptides. In some embodiments, the egg peptide composition comprises or consists about 24 egg peptides.

In some embodiments, the egg peptide composition comprises or consists from about 4 to about 12 egg peptides, from about 5 to about 11 egg peptides, from about 6 to about 10 egg peptides, from about 7 to about 9 egg peptides, or about 8 egg peptides. In some embodiments, the egg peptide composition comprises or consists from about 4 to about 12 egg peptides. In some embodiments, the egg peptide composition comprises or consists from about 5 to about 11 egg peptides. In some embodiments, the egg peptide composition comprises or consists from about 6 to about 10 egg peptides. In some embodiments, the egg peptide composition comprises or consists from about 7 to about 9 egg peptides. In some embodiments, the egg peptide composition comprises or consists about 8 egg peptides.

In some embodiments, the egg peptide composition comprises egg peptides derived from 1 to 7 egg proteins. In some embodiments, the egg peptide composition comprises egg peptides derived from 1 to 6 egg proteins. In some embodiments, the egg peptide composition comprises egg peptides derived from 1 to 5 egg proteins. In some embodiments, the egg peptide composition comprises egg peptides derived from 1 to 4 egg proteins. In some embodiments, the egg peptide composition comprises egg peptides derived from 1 to 3 egg proteins. In some embodiments, the egg peptide composition comprises egg peptides derived from 1 or 2 egg proteins. In some embodiments, the egg proteins are any one or more of: ovomucoid, ovalbumin, ovotransferrin, lysozyme, yolk serum albumin, YGP40, and YBG42. In some embodiments, the egg proteins are any one or more of: ovomucoid, ovalbumin, and ovotransferrin. In some embodiments, the egg proteins are any one or more of: ovomucoid, ovalbumin, and lysozyme. In some embodiments, the egg proteins are any one or more of: ovomucoid, ovalbumin, and yolk serum albumin. In some embodiments, the egg proteins are any one or more of: ovomucoid, ovalbumin, and YGP40. In some embodiments, the egg proteins are any one or more of: ovomucoid, ovalbumin, and YBG42. In some embodiments, the egg proteins are ovomucoid peptides and ovalbumin peptides.

In some embodiments, the egg peptide composition comprises or consists from about 26 to about 42 ovomucoid peptides, from about 28 to about 40 ovomucoid peptides, from about 30 to about 36 ovomucoid peptides, from about 31 to about 35 ovomucoid peptides, or about 34 ovomucoid peptides. In some embodiments, the egg peptide composition comprises or consists from about 26 to about 42 ovomucoid peptides. In some embodiments, the egg peptide composition comprises or consists from about 28 to about 40 ovomucoid peptides. In some embodiments, the egg peptide composition comprises or consists from about 30 to about 36 ovomucoid peptides. In some embodiments, the egg peptide composition comprises or consists from about 31 to about 35 ovomucoid peptides. In some embodiments, the egg peptide composition comprises or consists of about 34 ovomucoid peptides.

In some embodiments, the egg peptide composition comprises or consists from about 3 to about 9 ovomucoid peptides, from about 4 to about 8 ovomucoid peptides, from about 5 to about 7 ovomucoid peptides, about 5 ovomucoid peptides, or about 6 ovomucoid peptides. In some embodiments, the egg peptide composition comprises or consists from about 3 to about 9 ovomucoid peptides. In some embodiments, the egg peptide composition comprises or consists from about 4 to about 8 ovomucoid peptides. In some embodiments, the egg peptide composition comprises or consists from about 5 to about 7 ovomucoid peptides. In some embodiments, the egg peptide composition comprises or consists of about 5 ovomucoid peptides. In some embodiments, the egg peptide composition comprises or consists of about 6 ovomucoid peptides.

In some embodiments, the egg peptide composition comprises any one or more of the following ovomucoid peptides: OVM-001 (AEVDCSRFPNATDKE; SEQ ID NO: 1), OVM-003 (RFPNATDKEGKDVLV; SEQ ID NO: 2), OVM-004 (NATDKEGKDVLVCNK; SEQ ID NO: 3), OVM-005 (DKEGKDVLVCNKDLR; SEQ ID NO: 4), OVM-007 (VLVCNKDLRPICGTD; SEQ ID NO: 5), OVM-008 (CNKDLRPICGTDGVT; SEQ ID NO: 6), OVM-009 (DLRPICGTDGVTYTN; SEQ ID NO: 7), OVM-010 (PICGTDGVTYTNDCL; SEQ ID NO: 8), OVM-013 (YTNDCLLCAYSIEFG; SEQ ID NO: 9), OVM-014 (DCLLCAYSIEFGTNI; SEQ ID NO: 10), OVM-015 (LCAYSIEFGTNISKE; SEQ ID NO: 11), OVM-018 (TNISKEHDGECKETV; SEQ ID NO: 12), OVM-021 (ECKETVPMNCSSYAN; SEQ ID NO: 13), OVM-023 (PMNCSSYANTTSEDG; SEQ ID NO: 14), OVM-025 (YANTTSEDGKVMVLC; SEQ ID NO: 15), OVM-027 (EDGKVMVLCNRAFNP; SEQ ID NO: 16), OVM-028 (KVMVLCNRAFNPVCG; SEQ ID NO: 17), OVM-031 (FNPVCGTDGVTYDNE; SEQ ID NO: 18), OVM-032 (VCGTDGVTYDNECLL; SEQ ID NO: 19), OVM-033 (TDGVTYDNECLLCAH; SEQ ID NO: 20), OVM-036 (CLLCAHKVEQGASVD; SEQ ID NO: 21), OVM-038 (KVEQGASVDKRHDGG; SEQ ID NO: 22), OVM-040 (SVDKRHDGGCRKELA; SEQ ID NO: 23), OVM-041 (KRHDGGCRKELAAVS; SEQ ID NO: 24), OVM-042 (DGGCRKELAAVSVDC; SEQ ID NO: 25), OVM-044 (ELAAVSVDCSEYPKP; SEQ ID NO: 26), OVM-046 (VDCSEYPKPDCTAED; SEQ ID NO: 27), OVM-048 (PKPDCTAEDRPLCGS; SEQ ID NO: 28), OVM-050 (AEDRPLCGSDNKTYG; SEQ ID NO: 29), OVM-052 (CGSDNKTYGNKCNFC; SEQ ID NO: 30), OVM-054 (TYGNKCNFCNAVVES; SEQ ID NO: 31), OVM-055 (NKCNFCNAVVESNGT; SEQ ID NO: 32), OVM-056 (NFCNAVVESNGTLTL; SEQ ID NO: 33), and/or OVM-058 (VESNGTLTLSHFGKC; SEQ ID NO: 34). In some embodiments, the egg peptide composition comprises any one or more of the following ovomucoid peptides: OVM-014, OVM-004, OVM-009, OVM-032, OVM-031. In some embodiments, the egg peptide composition comprises any one or more of the following ovomucoid peptides: OVM-014, OVM-004, OVM-009, OVM-032, OVM-013.

In some embodiments, the egg peptide composition comprises or consists from about 13 to about 25 ovalbumin peptides, from about 15 to about 23 ovalbumin peptides, from about 17 to about 21 ovalbumin peptides, from about 18 to about 20 ovalbumin peptides, or about 19 ovalbumin peptides. In some embodiments, the egg peptide composition comprises or consists from about 13 to about 25 ovalbumin peptides. In some embodiments, the egg peptide composition comprises or consists from about 15 to about 23 ovalbumin peptides. In some embodiments, the egg peptide composition comprises or consists from about 17 to about 21 ovalbumin peptides. In some embodiments, the egg peptide composition comprises or consists from about 18 to about 20 ovalbumin peptides. In some embodiments, the egg peptide composition comprises or consists of about 19 ovalbumin peptides.

In some embodiments, the egg peptide composition comprises or consists from about 1 to about 7 ovalbumin peptides, from about 2 to about 6 ovalbumin peptides, from about 3 to about 5 ovalbumin peptides, about 4 ovalbumin peptides, or about 3 ovalbumin peptides. In some embodiments, the egg peptide composition comprises or consists from about 1 to about 7 ovalbumin peptides. In some embodiments, the egg peptide composition comprises or consists from about 2 to about 6 ovalbumin peptides. In some embodiments, the egg peptide composition comprises or consists from about 3 to about 5 ovalbumin peptides. In some embodiments, the egg peptide composition comprises or consists about 4 ovalbumin peptides. In some embodiments, the egg peptide composition comprises or consists of about 3 ovalbumin peptides.

In some embodiments, the egg peptide composition comprises any one or more of the following ovalbumin peptides: OVA-001 (MGSIGAASMEFCFDV; SEQ ID NO: 35), OVA-003 (ASMEFCFDVFKELKV; SEQ ID NO: 36), OVA-006 (FKELKVHHANENIFY; SEQ ID NO: 37), OVA-021 (DKLPGFGDSIEAQCG; SEQ ID NO: 38), OVA-028 (SSLRDILNQITKPND; SEQ ID NO: 39), OVA-040 (YLQCVKELYRGGLEP; SEQ ID NO: 40), OVA-053 (IIRNVLQPSSVDSQT; SEQ ID NO: 41), OVA-057 (SQTAXVLVNAIVFKG; SEQ ID NO: 42), OVA-059 (LVNAIVFKGLWEKAF; SEQ ID NO: 43), OVA-064 (KDEDTQAMPFRVTEQ; SEQ ID NO: 44), OVA-068 (TEQESKPVQMMYQIG; SEQ ID NO: 45), OVA-071 (MMYQIGLFRVASMAS; SEQ ID NO: 46), OVA-075 (MASEKMKILELPFAS; SEQ ID NO: 47), OVA-085 (LEQLESIINFEKLTE; SEQ ID NO: 48), OVA-090 (WTSSNVMEERKIKVY; SEQ ID NO: 49), OVA-095 (LPRMKMEEKYNLTSV; SEQ ID NO: 50), OVA-108 (LKISQAVHAAHAEIN; SEQ ID NO: 51), OVA-113 (EAGREVVGSAEAGVD; SEQ ID NO: 52), and/or OVA-120 (EFRADHPFLFCIKHI; SEQ ID NO: 53). In some embodiments, the egg peptide composition comprises any one or more of the following ovalbumin peptides: OVA-064, OVA-108, OVA-028.

In some embodiments, the egg peptide composition comprises or consists from about 0 to about 6 lysozyme peptides, from about 0 to about 5 lysozyme peptides, from about 1 to about 4 lysozyme peptides, from about 1 to about 3 lysozyme peptides, 1 lysozyme peptide, 2 lysozyme peptides, or 3 lysozyme peptides. In some embodiments, the egg peptide composition comprises or consists from about 0 to about 5 lysozyme peptides. In some embodiments, the egg peptide composition comprises or consists from about 1 to about 4 lysozyme peptides. In some embodiments, the egg peptide composition comprises or consists from about 1 to about 3 lysozyme peptides. In some embodiments, the egg peptide composition comprises or consists of 1 lysozyme peptide. In some embodiments, the egg peptide composition comprises or consists of 2 lysozyme peptides. In some embodiments, the egg peptide composition comprises or consists of 3 lysozyme peptides. In some embodiments, the egg peptide composition comprises or consists of 4 lysozyme peptides. In some embodiments, the egg peptide composition comprises or consists of 5 lysozyme peptides. In some embodiments, the egg peptide composition comprises or consists of 6 lysozyme peptides. In some embodiments, the egg peptide composition comprises or consists of no lysozyme peptides.

In some embodiments, the egg peptide composition comprises any one or more of the following lysozyme peptides: LYS-1 (AAAMKRHGLDNYRGY; SEQ ID NO: 54), LYS-2 (GYSLGNWVCAAKFES; SEQ ID NO: 55), LYS-3 (RNTDGSTDYGILQIN; SEQ ID NO: 56), LYS-4 (TDYGILQINSRWWCN; SEQ ID NO: 57), LYS-5 (WCNDGRTPGSRNLCN; SEQ ID NO: 58), and/or LYS-6 (WVAWRNRCKGTDVQA; SEQ ID NO: 59). In some embodiments, the egg peptide composition comprises any one or more of the following lysozyme peptides: LYS-1, LYS-3, and/or LYS-6.

In some embodiments, the egg peptide composition comprises or consists from about 0 to about 5 ovotransferrin peptides, from about 1 to about 4 ovotransferrin peptides, from about 1 to about 3 ovotransferrin peptides, 1 ovotransferrin peptide, 2 ovotransferrin peptides, or 3 ovotransferrin peptides. In some embodiments, the egg peptide composition comprises or consists from about 0 to about 5 ovotransferrin peptides. In some embodiments, the egg peptide composition comprises or consists from about 1 to about 4 ovotransferrin peptides. In some embodiments, the egg peptide composition comprises or consists from about 1 to about 3 ovotransferrin peptides. In some embodiments, the egg peptide composition comprises or consists of 1 ovotransferrin peptide. In some embodiments, the egg peptide composition comprises or consists of 2 ovotransferrin peptides. In some embodiments, the egg peptide composition comprises or consists of 3 ovotransferrin peptides. In some embodiments, the egg peptide composition comprises or consists of no ovotransferrin peptides.

In some embodiments, the egg peptide composition comprises any one or more of the following ovotransferrin peptides: OVT-1 (SSPEEKKCNNLRDLT; SEQ ID NO: 60), OVT-2 (LRDLTQQERISLTCV; SEQ ID NO: 61), OVT-3 (TIEQKLCRQCKGDPK; SEQ ID NO: 62), OVT-4 (DQLTPSPRENRIQWC; SEQ ID NO: 63), and/or OVT-5 (VMDYRECNLAEVPTH; SEQ ID NO: 64). In some embodiments, the egg peptide composition comprises any one or more of the following ovotransferrin peptides: OVT-1, OVT-3, and/or OVT-5.

In some embodiments, the egg peptide composition comprises or consists from about 0 to about 5 yolk serum albumin peptides, from about 1 to about 4 yolk serum albumin peptides, from about 1 to about 3 yolk serum albumin peptides, 1 yolk serum albumin peptide, 2 yolk serum albumin peptides, or 3 yolk serum albumin peptides. In some embodiments, the egg peptide composition comprises or consists from about 0 to about 5 yolk serum albumin peptides. In some embodiments, the egg peptide composition comprises or consists from about 1 to about 4 yolk serum albumin peptides. In some embodiments, the egg peptide composition comprises or consists from about 1 to about 3 yolk serum albumin peptides. In some embodiments, the egg peptide composition comprises or consists of 1 yolk serum albumin peptide. In some embodiments, the egg peptide composition comprises or consists of 2 yolk serum albumin peptides. In some embodiments, the egg peptide composition comprises or consists of 3 yolk serum albumin peptides. In some embodiments, the egg peptide composition comprises or consists of no yolk serum albumin peptides.

In some embodiments, the egg peptide composition comprises any one or more of the following yolk serum albumin peptides: sALB-1 (YQRPASDVICQEYQD SEQ ID NO: 65 or LRDSYGAMADCCSKA SEQ ID NO: 66), sALB-2 (LEKCCKTDNPAECYA; SEQ ID NO: 67), sALB-3 (KQETTPINDNVSQCC; SEQ ID NO: 68), sALB-4 (GVDTKYVPPPFNPDM; SEQ ID NO: 69), and/or sALB-5 (MVDKCCKQSDINTCFGE; SEQ ID NO: 70). In some embodiments, the egg peptide composition comprises any one or more of the following yolk serum albumin peptides: sALB-1, sALB-3 and/or sALB-4.

In some embodiments, the egg peptide composition comprises or consists from about 0 to about 5 YGP40 peptides, from about 1 to about 4 YGP40 peptides, from about 1 to about 3 YGP40 peptides, 1 YGP40 peptide, 2 YGP40 peptides, or 3 YGP40 peptides. In some embodiments, the egg peptide composition comprises or consists from about 0 to about 5 YGP40 peptides. In some embodiments, the egg peptide composition comprises or consists from about 1 to about 4 YGP40 peptides. In some embodiments, the egg peptide composition comprises or consists from about 1 to about 3 YGP40 peptides. In some embodiments, the egg peptide composition comprises or consists of 1 YGP40 peptide. In some embodiments, the egg peptide composition comprises or consists of 2 YGP40 peptides. In some embodiments, the egg peptide composition comprises or consists of 3 YGP40 peptides. In some embodiments, the egg peptide composition comprises or consists of no YGP40 peptides.

In some embodiments, the egg peptide composition comprises any one or more of the following YGP40 peptides: YGP40-1 (NYSMPANCYHILVQD; SEQ ID NO: 71), YGP40-2 (VQDCSSELKFLVMMK; SEQ ID NO: 72), YGP40-3 (CAKGCSATKTTPVTV; SEQ ID NO: 73), YGP40-4 (CSATKTTPVTVGFHC; SEQ ID NO: 74), and/or YGP40-5 (FHCLPADSANSLTDK; SEQ ID NO: 75). In some embodiments, the egg peptide composition comprises any one or more of the following YGP40 peptides: YGP40-1 and/or YGP40-5.

In some embodiments, the egg peptide composition comprises or consists from about 0 to about 5 YBG42 peptides, from about 1 to about 4 YBG42 peptides, from about 1 to about 3 YBG42 peptides, 1 YBG42 peptide, 2 YBG42 peptides, or 3 YBG42 peptides. In some embodiments, the egg peptide composition comprises or consists from about 0 to about 5 YBG42 peptides. In some embodiments, the egg peptide composition comprises or consists from about 1 to about 4 YBG42 peptides. In some embodiments, the egg peptide composition comprises or consists from about 1 to about 3 YBG42 peptides. In some embodiments, the egg peptide composition comprises or consists of 1 YBG42 peptide. In some embodiments, the egg peptide composition comprises or consists of 2 YBG42 peptides. In some embodiments, the egg peptide composition comprises or consists of 3 YBG42 peptides. In some embodiments, the egg peptide composition comprises or consists of no YBG42 peptides.

In some embodiments, the egg peptide composition comprises any one or more of the following YBG42 peptides: YGP42-1 (VDHQSLSREVHINTS; SEQ ID NO: 76), YGP42-2 (GVCGNNDREKHNELL; SEQ ID NO: 77), and/or YGP42-3 (SSDKKSASEDVVESV; SEQ ID NO: 78). In some embodiments, the egg peptide composition comprises the following YBG42 peptide: YGP42-1.

In some embodiments, the egg peptide composition comprises or consists of the following egg peptides: OVM-001, OVM-003, OVM-004, OVM-005, OVM-007, OVM-008, OVM-009, OVM-010, OVM-013, OVM-014, OVM-015, OVM-018, OVM-021, OVM-023, OVM-025, OVM-027, OVM-028, OVM-031, OVM-032, OVM-033, OVM-036, OVM-038, OVM-040, OVM-041, OVM-042, OVM-044, OVM-046, OVM-048, OVM-050, OVM-052, OVM-054, OVM-055, OVM-056, OVM-058, OVA-001, OVA-003, OVA-006, OVA-021, OVA-028, OVA-040, OVA-053, OVA-057, OVA-059, OVA-064, OVA-068, OVA-071, OVA-075, OVA-085, OVA-090, OVA-095, OVA-108, OVA-113, OVA-120, OVT-1, OVT-3, OVT-5, LYS-1, LYS-3, LYS-6, sALB-1, sALB-4, sALB-5, YGP40-1, YGP40-5, and YGP42-1.

In some embodiments, the egg peptide composition comprises or consists of the following egg peptides: OVT-1, OVT-2, OVT-3, OVT-4, OVT-5, LYS-1, LYS-2, LYS-3, LYS-4, LYS-5, LYS-6, sALB-1, sALB-2, sALB-3, sALB-4, sALB-5, YGP42-1, YGP42-2, YGP42-3, YGP40-1, YGP40-2, YGP40-3, YGP40-4, and YGP40-5.

In some embodiments, the egg peptide composition comprises or consists of the following egg peptides: OVM-014, OVM-004, OVM-009, OVM-032, OVA-064, OVA-108, OVM-013, OVA-028, OVM-031, LYS-1, and LYS-3.

In some embodiments, the egg allergen peptide is any one or more of: OVM-014, OVM-004, OVM-009, OVM-032, OVA-064, OVA-108, OVM-013, and OVA-028.

In some embodiments, any of the egg peptides described herein can have one to four conservative amino acid substitutions therein. In some embodiments, any of the egg peptides described herein can have one to three conservative amino acid substitutions therein. In some embodiments, any of the egg peptides described herein can have two to four conservative amino acid substitutions therein. In some embodiments, any of the egg peptides described herein can have one conservative amino acid substitution therein. In some embodiments, any of the egg peptides described herein can have two conservative amino acid substitutions therein. In some embodiments, any of the egg peptides described herein can have three conservative amino acid substitutions therein. In some embodiments, any of the egg peptides described herein can have four conservative amino acid substitutions therein.

Conservative amino acid substitutions are most often classified on the basis of the amino acid structure and the general chemical characteristics of their side chains (R groups). For example, aliphatic amino acids include glycine, alanine, valine, leucine, and isoleucine, and each of these amino acids can be substituted for one another. Hydroxyl or sulfur/selenium-containing amino acids include serine, cysteine, selenocysteine, threonine, and methionine, and each of these amino acids can be substituted for one another. Aromatic amino acids include phenylalanine, tyrosine, and tryptophan, and each of these amino acids can be substituted for one another. Basic amino acids include histidine, lysine, and arginine, and each of these amino acids can be substituted for one another. Acidic or amide-containing amino acids include aspartate, glutamate, asparagine, and glutamine, and each of these amino acids can be substituted for one another.

In some embodiments, each egg peptide is coupled to a solid support. In some embodiments, the solid support is a microsphere bead, a glass array, a silicone array, a membrane, or a microtiter plate. In some embodiments, the solid support is a glass array. In some embodiments, the solid support is a silicone array. In some embodiments, the solid support is a membrane. In some embodiments, the solid support is a microtiter plate. In some embodiments, the solid support is a microsphere bead. In some embodiments, the microsphere bead is an avidin-coupled microsphere bead. In some embodiments, the bead is a Luminex bead such as Mag™ Avidin bead or LumAvidin® bead. In some embodiments, each of the solid supports is coupled to a single egg peptide.

In some embodiments, each of the egg peptides is coupled to the solid support by a linker-spacer. In some embodiments, the linker-spacer comprises a linker chosen from biotin, a thiol, a hydrazine, and an amine. In some embodiments, the linker is biotin. In some embodiments, the linker is a thiol. In some embodiments, the linker is a hydrazine. In some embodiments, the linker is an amine. In some embodiments, the linker-spacer comprises a spacer chosen from a polypeptide, an oligonucleotide, an alkyl group, and a polyethylene glycol (PEG) group. In some embodiments, the spacer is a polypeptide. In some embodiments, the spacer is an oligonucleotide. In some embodiments, the spacer is an alkyl group. In some embodiments, the alkyl group is a C₁-C₁₈alkyl group or a C₃-C₂alkyl group. In some embodiments, the spacer is a PEG group. In some embodiments, the PEG group is PEG1 to PEG18. In some embodiments, the PEG group is PEG12. In some embodiments, the spacer is an alkyl group or a PEG group. In some embodiments, the C-terminus of a plurality of the egg peptides is coupled to the solid support by the linker-spacer. In some embodiments, the C-terminus of each of the egg peptides is coupled to the solid support by the linker-spacer. In some embodiments, the N-terminus of a plurality of the egg peptides is coupled to the solid support by the linker-spacer. In some embodiments, the N-terminus of each of the egg peptides is coupled to the solid support by the linker-spacer. In some embodiments, the C-terminus of each of the egg peptides is coupled to the solid support by a biotin-PEG12 linker-spacer.

In some embodiments, the egg peptides can be coupled to the solid support, with each component having one half of a pair of click chemistry linkers. For example, one of the egg peptides and solid support can have one click chemistry linker, while the other of the egg peptides and solid support can have a corresponding click chemistry linker. Examples of click chemistry linker pairs include, but are not limited to, azide-DBCO, amine-NHS ester, and thiol-malamide.

The present disclosure also provides methods for diagnosing an egg allergy in a subject. The methods comprise contacting any one or more of the egg peptides described herein (such as any of the combinations of egg peptides within any of the egg peptide compositions described herein) coupled to a solid support with a biological sample obtained from the subject. The contacting occurs under conditions sufficient to permit binding of one or more allergy associated immunoglobulins (AAIs) in the biological sample to the one or more egg peptides to form AAI-peptide-solid support complexes. The methods also comprise contacting the AAI-peptide-solid support complexes with an AAI-specific labeling reagent to form labeling reagent-AAI-peptide-solid support complexes. The methods also comprise measuring the binding of the AAI-specific labeling reagent to each AAI-peptide-solid support complex. When the combined binding for each egg peptide in the AAI-peptide solid support complex to the AAI-specific labeling reagent is greater than a threshold value, the subject is allergic to eggs. When the combined binding for each egg peptide in the AAI-peptide solid support complex to the AAI-specific labeling reagent is equal to or less than the threshold value, the subject is not allergic to eggs. The steps described herein comprise an assay for detecting the presence of specific AAIs in the biological sample to the one or more egg peptides.

Any one or more of the egg peptides (such as any of the combinations of egg peptides within any of the egg peptide compositions described herein) coupled to any of the solid supports described herein can be used. For example, in some embodiments, the solid supports can be coupled to the following egg peptides: OVM-014, OVM-004, OVM-009, OVM-032, OVA-064, OVA-108, OVM-013, OVA-028, OVM-031, LYS-1, and LYS-3. Also for example, in some embodiments, the solid supports can be coupled to the following egg peptides: OVM-014, OVM-004, OVM-009, OVM-032, OVA-064, OVA-108, OVM-013, and OVA-028. Any of the egg peptides can have conservative amino acid substitutions therein, as described herein. In some embodiments, the egg peptides are coupled to a microsphere bead. Each of the egg peptides can be coupled to the solid support by any of the linker-spacers described herein. Each of the egg peptides can be coupled to the solid support by their C-terminal or N-terminal ends as described herein.

The biological sample can be any biological sample obtained from a subject. In some embodiments, the biological sample is chosen from serum, plasma, saliva, or a buccal swab. In some embodiments, the biological sample is serum or plasma. In some embodiments, the biological sample is serum. In some embodiments, the biological sample is plasma. In some embodiments, the biological sample is saliva. In some embodiments, the biological sample is a buccal swab.

The AAIs that may be present in the biological sample from a subject may include any one or more of IgM, IgA, IgD, IgG, and/or IgE. In some embodiments, the AAI in the biological sample is IgM, IgA, and/or IgD. In some embodiments, the AAI in the biological sample is IgG and/or IgE. In some embodiments, the AAI in the biological sample is IgE.

In some embodiments, the AAI-specific labeling reagent is a detectably labeled anti-human antibody. In some embodiments, the detectably labeled anti-human antibody is detectably labeled anti-human IgA antibody. In some embodiments, the detectably labeled anti-human antibody is detectably labeled anti-human IgD antibody. In some embodiments, the detectably labeled anti-human antibody is detectably labeled anti-human IgM antibody. In some embodiments, the detectably labeled anti-human antibody is detectably labeled anti-human IgG antibody. In some embodiments, the detectably labeled anti-human antibody is detectably labeled anti-human IgE antibody.

In some embodiments, the detectable label of the AAI-specific labeling reagent is chosen from phycoerythrin (PE), a cyanine dye, a fluorescent dye, an infrared dye, a chromogenic dye, an enzyme label, and a radioactive label. In some embodiments, the detectable label of the AAI-specific labeling reagent is PE. In some embodiments, the detectable label of the AAI-specific labeling reagent is a cyanine dye. In some embodiments, the cyanine dye is Cy3 or Cy5. In some embodiments, the detectable label of the AAI-specific labeling reagent is a fluorescent dye. In some embodiments, the fluorescent dye is Texas Red or Alexa-fluor. In some embodiments, the detectable label of the AAI-specific labeling reagent is an infrared (IR) dye. In some embodiments, the detectable label of the AAI-specific labeling reagent is a chromogenic dye. In some embodiments, the detectable label of the AAI-specific labeling reagent is an enzyme label. In some embodiments, the detectable label of the AAI-specific labeling reagent is a radioactive label. In some embodiments, the enzyme label is horse radish peroxidase (HRP) or alkaline phosphatase. In some embodiments, the detectable label of the AAI-specific labeling reagent is HRP. In some embodiments, the detectable label of the AAI-specific labeling reagent is alkaline phosphatase. In some embodiments, the AAI-specific labeling reagent is a PE-labeled anti-human IgE antibody. In some embodiments, a single detectable label can generally be used for universal detection of all complexes.

In some embodiments, the anti-human AAI antibody may be conjugated to a reporter moiety that is not directly detectable, so specific binding of a second, directly detectable reporter moiety to the labeling reagent is necessary for analysis of binding. For example, a biotin-conjugated anti-AAI antibody can be used in combination with a streptavidin-conjugated fluorescent dye for detection of the biotin-conjugated anti-AAI. Examples of indirectly-detectable reporter moieties include biotin, digoxigenin, and other haptens that are detectable upon subsequent binding of a secondary antibody (e.g., anti-digoxigenin) or other binding partner (e.g., streptavidin) which is labeled for direct detection.

In some embodiments, the measuring of the binding of the AAI-specific labeling reagent to each AAI-peptide-solid support complex is carried out by a point of care device. In some embodiments, the point of care device is a multiplex peptide-bead flow cytometric analysis device or a lateral flow assay device. In some embodiments, the detectable label can be observed via silver staining, quantum dots, or refraction methodologies.

Any of the foregoing embodiments may be in the form of a microarray immunoassay, wherein any one or more of the egg peptides is bound to a separate well of a microtiter plate and reacted with a biological sample to bind AAI. The egg peptides may also be used in a lateral flow immunoassay format, wherein each peptide is immobilized in a discrete area on a porous or chromatographic support, and the serum or plasma is wicked through the support to contact the peptides for binding of AAI to the egg peptides. In this assay, the AAI-specific labeling reagent may comprise a chromophore or dye conjugated to anti-AAI antibody. The labeling reagent is also wicked through the support to contact the peptide-AAI complexes for binding of the labeling reagent to the complex, which indicates the presence or absence in the serum or plasma of an antibody to the egg peptide immobilized at each discrete location of the support.

Any of the foregoing embodiments may also be in the form of a flow cytometry assay in which each egg peptide is coupled to a separately identifiable solid support suitable for analysis by flow cytometry, such as a bead. In some embodiments, the bead with the coupled egg peptide is contacted with the biological sample of a subject to bind any peptide-specific AAI that is bound to the bead via the peptide, thus forming a peptide-AAI complex on the bead. An AAI-specific labeling reagent comprising, for example, a fluorescent reporter moiety, is then bound to the peptide-AAI complexes and the beads are analyzed quantitatively or qualitatively by flow cytometry. This detects fluorescence from the bound labeling reagent associated with each bead to which the egg peptide is coupled.

In some embodiments, the flow cytometry assay may be a multiplex assay, such as provided by Luminex, which uses a microsphere array platform for quantitation and detection of peptides and proteins. Each of the egg peptides is bound to a set of beads with the same or different spectral properties which can be used to quantify the associated egg peptide bound to AAI by flow cytometry. The sets of beads are then contacted with the biological sample of a subject to bind peptide-recognizing AAI to each bead to form a peptide-AAI complex on the bead, and an AAI-specific labeling reagent comprising, for example, a fluorescent reporter moiety bound to the AAI of the complex. The beads are analyzed by monitoring the spectral properties of each bead and the amount of associated fluorescence from the bound labeling reagent. This process allows quantification of the egg peptide on the bead, and the presence or absence of AAI that is reactive to it. Results of the assay are interpreted as discussed herein.

A particularly useful quantitative assay for use in any of the methods described herein is a multiplex peptide-bead assay for flow cytometric analysis, such as the LUMINEX exMAP multiplex bead assay, which is a high-throughput alternative to the ELISA. In this assay, polystyrene beads (microspheres) dyed with distinct proportions of red and near-infrared fluorophores are used as the solid support. The peptides may be chemically linked to the beads or bound thereto through peptide-specific capture antibodies coated on the beads. The proportions of the fluorophores define a “spectral address” for each bead population that can be identified by a flow cytometer using digital signal processing. Detection of a third fluorescence color is used for measurement of the fluorescence intensity of the reporter moiety of the labeling reagent bound to the bead. Multiple analytes can be detected simultaneously by binding each egg peptide to a bead having a specific “spectral address.” Contacting the beads with a biological sample containing AAI that are specific for the egg peptide bound to it is followed by addition of anti-human AAI antibodies conjugated to a reporter moiety. In some embodiments, the reporter moiety of the anti-human AAI is biotin and binding to phycoerythyrin (PE)-conjugated streptavidin provides the fluorescent signal for detection. Following binding of the labeling reagent, the beads are analyzed on a dual-laser flow-based detection instrument, such as the LUMINEX 200 or Bio-Rad BIO-PLEX analyzer. One laser classifies the bead and identifies the peptide bound to it. The second laser determines the magnitude of the reporter-derived signal, which is in direct proportion to the amount of bound serum or plasma AAI.

In some embodiments, measuring the binding of the AAI-specific labeling reagent to each AAI-peptide-solid support complex comprises measuring the mean fluorescent intensity (MFI) of each AAI-specific labeling reagent. When the combined MFI binding for each egg peptide in the AAI-peptide solid support complex to the AAI-specific labeling reagent is greater than a threshold value, the subject is allergic to eggs. When the combined MFI for each egg peptide in the AAI-peptide solid support complex to the AAI-specific labeling reagent is equal to or less than the threshold value, the subject is not allergic to eggs. An epitope (i.e., peptide) is considered detectable and may contribute to an allergic reaction if it is above the limit of detection as determined by: Limit of Detection=(Mean of a Negative Control)+1.645×(Standard Deviation of Negative Control)+1.645×(Standard Deviation of a low positive sample). For example, for an egg epitope (i.e., peptide), if: (Mean of negative Control)=2 MFI, (Standard Deviation of Negative Control)=0.1, and (Standard Deviation of Low Positive)=0.1, then the Limit of Detection is 2.329 MFI, where MFI=Mean Fluorescent Units. Therefore, any value above 2.329 would be considered positive for that epitope (i.e., peptide). In some embodiments, the AAI-specific labeling reagent is any of the detectably labeled anti-human antibodies described herein. In some embodiments, the MFI of each AAI-specific labeling reagent is background subtracted.

In some embodiments, the methods further comprise performing or having performed a Skin Prick Test (SPT) and/or a total egg specific IgE (sIgE) test. In some embodiments, the methods further comprise performing or having performed a SPT. In some embodiments, the methods further comprise performing or having performed an sIgE test. In some embodiments, when the SPT is ≤3 mm and/or the sIgE is ≤0.10 kU/L then the subject is not allergic to eggs, and when the SPT is ≥18 mm and/or the sIgE is ≥18 kU/L then the subject is allergic to eggs.

The present disclosure also provides methods for detecting development of clinical tolerance to eggs in a subject that is allergic to eggs. The methods comprise contacting any one or more of the egg peptides (such as any of the combinations of egg peptides within any of the egg peptide compositions described herein) coupled to a solid support with a biological sample obtained from the subject under conditions sufficient to permit binding of one or more allergy associated immunoglobulins (AAIs) in the biological sample to the egg peptides to form AAI-peptide-solid support complexes. The AAI-peptide-solid support complexes are then contacted with an AAI-specific labeling reagent to form labeling reagent-AAI-peptide-solid support complexes. The methods comprise measuring the binding of the AAI-specific labeling reagent to each AAI-peptide-solid support complex. The methods also comprise comparing the binding of the AAI-specific labeling reagent to each AAI-peptide-solid support complex to the binding of the AAI-specific labeling reagent to each AAI-peptide-solid support complex from a biological sample previously obtained from the subject. When the combined binding for each egg peptide in the AAI-peptide solid support complex to the AAI-specific labeling reagent is greater than or equal to the combined binding for each egg peptide in the AAI-peptide solid support complex to the AAI-specific labeling reagent for the previously obtained biological sample, the subject has not established clinical tolerance to eggs. When the combined binding for each egg peptide in the AAI-peptide solid support complex to the AAI-specific labeling reagent is less than the combined binding for each egg peptide in the AAI-peptide solid support complex to the AAI-specific labeling reagent for the previously obtained biological sample, the subject has established clinical tolerance to eggs.

Any of the egg peptides (such as any of the combinations of egg peptides within any of the egg peptide compositions described herein) coupled to any of the solid supports described herein can be used. For example, in some embodiments, the solid supports can be coupled to the following egg peptides: OVM-014, OVM-004, OVM-009, OVM-032, OVA-064, OVA-108, OVM-013, OVA-028, OVM-031, LYS-1, and LYS-3. Also for example, in some embodiments, the solid supports can be coupled to the following egg peptides: OVM-014, OVM-004, OVM-009, OVM-032, OVA-064, OVA-108, OVM-013, and OVA-028. Any of the egg peptides can have conservative amino acid substitutions therein, as described herein. In some embodiments, the egg peptides are coupled to a microsphere bead. Each of the egg peptides can be coupled to the solid support by any of the linker-spacers described herein. Each of the egg peptides can be coupled to the solid support by their C-terminal or N-terminal ends as described herein.

The biological sample can be any biological sample obtained from a subject. In some embodiments, the biological sample is chosen from serum, plasma, saliva, or a buccal swab. In some embodiments, the biological sample is serum or plasma. In some embodiments, the biological sample is serum. In some embodiments, the biological sample is plasma. In some embodiments, the biological sample is saliva. In some embodiments, the biological sample is a buccal swab.

The AAIs that may be present in the biological sample from a subject may include any one or more of IgM, IgA, IgD, IgG, and/or IgE. In some embodiments, the AAI in the biological sample is IgM, IgA, and/or IgD. In some embodiments, the AAI in the biological sample is IgG and/or IgE. In some embodiments, the AAI in the biological sample is IgE.

In some embodiments, the AAI-specific labeling reagent is a detectably labeled anti-human antibody. In some embodiments, the detectably labeled anti-human antibody is detectably labeled anti-human IgA antibody. In some embodiments, the detectably labeled anti-human antibody is detectably labeled anti-human IgD antibody. In some embodiments, the detectably labeled anti-human antibody is detectably labeled anti-human IgM antibody. In some embodiments, the detectably labeled anti-human antibody is detectably labeled anti-human IgG antibody. In some embodiments, the detectably labeled anti-human antibody is detectably labeled anti-human IgE antibody.

In some embodiments, the detectable label of the AAI-specific labeling reagent is chosen from PE, a cyanine dye, a fluorescent dye, an infrared dye, a chromogenic dye, an enzyme label, and a radioactive label. In some embodiments, the detectable label of the AAI-specific labeling reagent is PE. In some embodiments, the detectable label of the AAI-specific labeling reagent is a cyanine dye. In some embodiments, the cyanine dye is Cy3 or Cy5. In some embodiments, the detectable label of the AAI-specific labeling reagent is a fluorescent dye. In some embodiments, the fluorescent dye is Texas Red or Alexa-fluor. In some embodiments, the detectable label of the AAI-specific labeling reagent is an IR dye. In some embodiments, the detectable label of the AAI-specific labeling reagent is a chromogenic dye. In some embodiments, the detectable label of the AAI-specific labeling reagent is an enzyme label. In some embodiments, the detectable label of the AAI-specific labeling reagent is a radioactive label. In some embodiments, the enzyme label is HRP or alkaline phosphatase. In some embodiments, the detectable label of the AAI-specific labeling reagent is HRP. In some embodiments, the detectable label of the AAI-specific labeling reagent is alkaline phosphatase. In some embodiments, the AAI-specific labeling reagent is a PE-labeled anti-human IgE antibody. In some embodiments, a single detectable label can generally be used for universal detection of all complexes.

In some embodiments, the anti-human AAI antibody may be conjugated to a reporter moiety that is not directly detectable, so specific binding of a second, directly detectable reporter moiety to the labeling reagent is necessary for analysis of binding. For example, a biotin-conjugated anti-AAI antibody can be used in combination with a streptavidin-conjugated fluorescent dye for detection of the biotin-conjugated anti-AAI. Examples of indirectly-detectable reporter moieties include biotin, digoxigenin, and other haptens that are detectable upon subsequent binding of a secondary antibody (e.g., anti-digoxigenin) or other binding partner (e.g., streptavidin) which is labeled for direct detection.

In some embodiments, the measuring of the binding of the AAI-specific labeling reagent to each AAI-peptide-solid support complex is carried out by a point of care device. In some embodiments, the point of care device is a multiplex peptide-bead flow cytometric analysis device or a lateral flow assay device. In some embodiments, the detectable label can be observed via silver staining, quantum dots, or refraction methodologies.

Any of the foregoing embodiments may be in the form of a microarray immunoassay, wherein each of the one or more egg peptides is bound to a separate well of a microtiter plate and reacted with a biological sample to bind AAI. The egg peptides may also be used in a lateral flow immunoassay format, wherein each egg peptide is immobilized in a discrete area on a porous or chromatographic support, and the serum or plasma is wicked through the support to contact the egg peptides for binding of AAI to the egg peptides. In this assay, the AAI-specific labeling reagent may comprise a chromophore or dye conjugated to anti-AAI antibody. The labeling reagent is also wicked through the support to contact the peptide-AAI complexes for binding of the labeling reagent to the complex, which indicates the presence or absence in the serum or plasma of an antibody to the egg peptide immobilized at each discrete location of the support.

Any of the foregoing embodiments may also be in the form of a flow cytometry assay in which each egg peptide is coupled to a separately identifiable solid support suitable for analysis by flow cytometry, such as a bead. In some embodiments, the bead with the coupled peptide is contacted with the biological sample of a subject to bind any peptide-specific AAI that is bound to the bead via the peptide, thus forming a peptide-AAI complex on the bead. An AAI-specific labeling reagent comprising, for example, a fluorescent reporter moiety, is then bound to the peptide-AAI complexes and the beads are analyzed quantitatively or qualitatively by flow cytometry. This detects fluorescence from the bound labeling reagent associated with each bead to which the egg peptide is coupled.

In some embodiments, the flow cytometry assay may be a multiplex assay, such as provided by Luminex, which uses a microsphere array platform for quantitation and detection of peptides and proteins. Each of the egg peptides is bound to a set of beads with the same or different spectral properties which can be used to quantify the associated egg peptide bound to AAI by flow cytometry. The sets of beads are then contacted with the biological sample of a subject to bind peptide-recognizing AAI to each bead to form a peptide-AAI complex on the bead, and an AAI-specific labeling reagent comprising, for example, a fluorescent reporter moiety bound to the AAI of the complex. The beads are analyzed by monitoring the spectral properties of each bead and the amount of associated fluorescence from the bound labeling reagent. This process allows quantification of the egg peptide on the bead, and the presence or absence of AAI that is reactive to it. Results of the assay are interpreted as discussed herein.

A particularly useful quantitative assay for use in any of the methods described herein is a multiplex peptide-bead assay for flow cytometric analysis, such as the LUMINEX exMAP multiplex bead assay, which is a high-throughput alternative to the ELISA. In this assay, polystyrene beads (microspheres) dyed with distinct proportions of red and near-infrared fluorophores are used as the solid support. The egg peptides may be chemically linked to the beads or bound thereto through peptide-specific capture antibodies coated on the beads. The proportions of the fluorophores define a “spectral address” for each bead population that can be identified by a flow cytometer using digital signal processing. Detection of a third fluorescence color is used for measurement of the fluorescence intensity of the reporter moiety of the labeling reagent bound to the bead. Multiple analytes can be detected simultaneously by binding each egg peptide to a bead having a specific “spectral address.” Contacting the beads with a biological sample containing AAI that are specific for the egg peptide bound to it is followed by addition of anti-human AAI antibodies conjugated to a reporter moiety. In some embodiments, the reporter moiety of the anti-human AAI is biotin and binding to PE-conjugated streptavidin provides the fluorescent signal for detection. Following binding of the labeling reagent, the beads are analyzed on a dual-laser flow-based detection instrument, such as the LUMINEX 200 or Bio-Rad BIO-PLEX analyzer. One laser classifies the bead and identifies the egg peptide bound to it. The second laser determines the magnitude of the reporter-derived signal, which is in direct proportion to the amount of bound serum or plasma AAI.

In some embodiments, measuring the binding of the AAI-specific labeling reagent to each AAI-peptide-solid support complex comprises measuring the MFI of each AAI-specific labeling reagent as described herein. The methods also comprise comparing the binding of the AAI-specific labeling reagent to each AAI-peptide-solid support complex to the binding of the AAI-specific labeling reagent to each AAI-peptide-solid support complex from a biological sample previously obtained from the subject. When the combined binding for each egg peptide in the AAI-peptide solid support complex to the AAI-specific labeling reagent is greater than or equal to the combined binding for each egg peptide in the AAI-peptide solid support complex to the AAI-specific labeling reagent for the previously obtained biological sample, the subject has not established clinical tolerance to eggs. When the combined binding for each egg peptide in the AAI-peptide solid support complex to the AAI-specific labeling reagent is less than the combined binding for each egg peptide in the AAI-peptide solid support complex to the AAI-specific labeling reagent for the previously obtained biological sample, the subject has established clinical tolerance to eggs. In some embodiments, the MFI of each AAI-specific labeling reagent is background subtracted.

The age of a subject undergoing examination for development of clinical tolerance can be from about 2 years old to 17 years old. In some embodiments, the subject is less than about one year old, less than about 2 years old, less than about 3 years old, less than about 4 years old, less than about 5 years old, or less than about 6 years old. In some embodiments, the subject is less than about one year old. In some embodiments, the subject is less than about 2 years old. In some embodiments, the subject is less than about 3 years old. In some embodiments, the subject is less than about 4 years old. In some embodiments, the subject is less than about 5 years old. In some embodiments, the subject is less than about 6 years old.

In some embodiments, the initial detection of development of clinical tolerance can be used to predict if a subject will either develop a natural tolerance to the allergy or be responsive to therapy. In some embodiments, an allergic subject is exposed to the immunogen (immunotherapy) prior to analyzing the initial profile. If at the subsequent time-point there is a reduction of at least 2-fold in serum concentration of all AAIs to the one or more egg peptides (such as any of the combinations of egg peptides within any of the egg peptide compositions described herein) in the initial profile, it is likely that the subject will develop either clinical or natural tolerance to eggs.

The present disclosure also provides methods of desensitizing an infant to one or more of the egg peptides (such as any of the combinations of egg peptides within any of the egg peptide compositions described herein) to induce tolerance or non-allergy to eggs. In some embodiments, the methods comprise administering any of the egg peptides (such as any of the combinations of egg peptides within any of the egg peptide compositions described herein) to the infant. Any of the egg peptides described herein can be administered. For example, in some embodiments, the following egg peptides can be administered: OVM-014, OVM-004, OVM-009, OVM-032, OVA-064, OVA-108, OVM-013, OVA-028, OVM-031, LYS-1, and LYS-3. Also for example, in some embodiments, the following egg peptides can be administered: OVM-014, OVM-004, OVM-009, OVM-032, OVA-064, OVA-108, OVM-013, and OVA-028. Any of the egg peptides can have conservative amino acid substitutions therein, as described herein. These peptides may have certain utility for the desensitization of egg allergy either individually, in combination, or in combination with other therapeutic approaches.

The egg peptides can be administered via an oral, sublingual, intradermal, sub-cutaneous, inhaled, or epicutaneous route to induce desensitization. The amount of total peptide or individual peptide can be about 1 gram or less per dose. The age of a subject undergoing desensitization can be less than about one year old, less than about 2 years old, less than about 3 years old, less than about 4 years old, less than about 5 years old, or less than about 6 years old. In some embodiments, the age of a subject undergoing desensitization is less than about one year old. In some embodiments, the age of a subject undergoing desensitization is less than about 2 years old. In some embodiments, the age of a subject undergoing desensitization is less than about 3 years old. In some embodiments, the age of a subject undergoing desensitization is less than about 4 years old. In some embodiments, the age of a subject undergoing desensitization is less than about 5 years old. In some embodiments, the age of a subject undergoing desensitization is less than about 6 years old.

The present disclosure also provides kits for carrying out any of the methods described herein. In some embodiments, the kit comprises a solid support coupled to any one or more of the egg peptides described herein (such as any of the combinations of egg peptides within any of the egg peptide compositions described herein). For example, in some embodiments, the kit can comprise the following egg peptides: OVM-014, OVM-004, OVM-009, OVM-032, OVA-064, OVA-108, OVM-013, OVA-028, OVM-031, LYS-1, and LYS-3. Also for example, in some embodiments, the kit can comprise the following egg peptides: OVM-014, OVM-004, OVM-009, OVM-032, OVA-064, OVA-108, OVM-013, and OVA-028. Any of the egg peptides can have conservative amino acid substitutions therein, as described herein. The kits also comprise an allergy associated immunoglobulin (AAI)-specific labeling reagent.

The kits described herein may also comprise additional components. In some embodiments, the kit further comprises instructions for use. In some embodiments, the kit further comprises one or more of a binding buffer, a wash buffer, a detection buffer, a non-allergic control sample, a negative buffer control sample, and an allergic positive control sample. In some embodiments, peptides containing non-reactive epitopes of egg proteins can be used as negative controls.

The peptides coupled to the solid support can be any of the egg peptides described herein and can be coupled to the solid support by any of the means described herein. The solid supports can be any of the solid supports described herein. The AAI-specific labeling reagents can be any of the AAI-specific labeling reagents described herein. The detectable label for any of the AAI-specific labeling reagents can be any of the detectable labels described herein.

The subject matter described herein has many advantages. First, the methods identify allergic and non-allergic subjects with confidence (e.g., PPV=95% and NPV=92%). Second, the methods may allow medical practitioners to de-list existing patients, possibly up to two-thirds, with indeterminate test results and/or ambiguous clinical history, significantly reduce overdiagnosis by minimizing false positive, and improve quality of life issues (e.g., anxiety, bullying, need for egg-free environments). Third, the methods provide a better way to monitor a subject's disease status over time, to monitor allergy outgrowth over time, and to provide a quantitative measure of disease status over time. Fourth, the methods can be used to provide a better treatment guidance. For example, the methods can be used to enhance the decision for or against oral food challenge (OFC) or therapy, optimize the use of OFC, and identify appropriate candidates for therapy with confidence. Fifth, the methods described herein reduce costs. For example, the methods can reduce the need for additional testing (e.g., OFC, sIgE, and component-resolved diagnosis (CRD), eliminate costs associated with food allergy by de-labeling those misdiagnosed, eliminate costs associated with unnecessary immunotherapy by de-labeling, de-labeled subjects will no longer need EpiPens purchasing and restocking, noon further need to purchase non-egg foods, and a reduction in other costs such as, for example, sick-days in the work place.

In order that the subject matter disclosed herein may be more efficiently understood, examples are provided below. It should be understood that these examples are for illustrative purposes only and are not to be construed as limiting the claimed subject matter in any manner.

### EXAMPLES

### Example 1: Materials and Methods

### Study Design

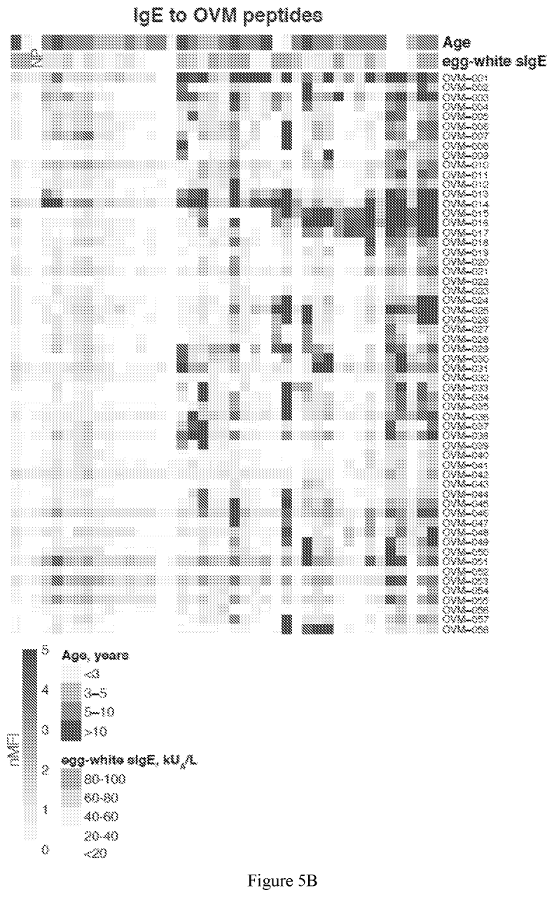

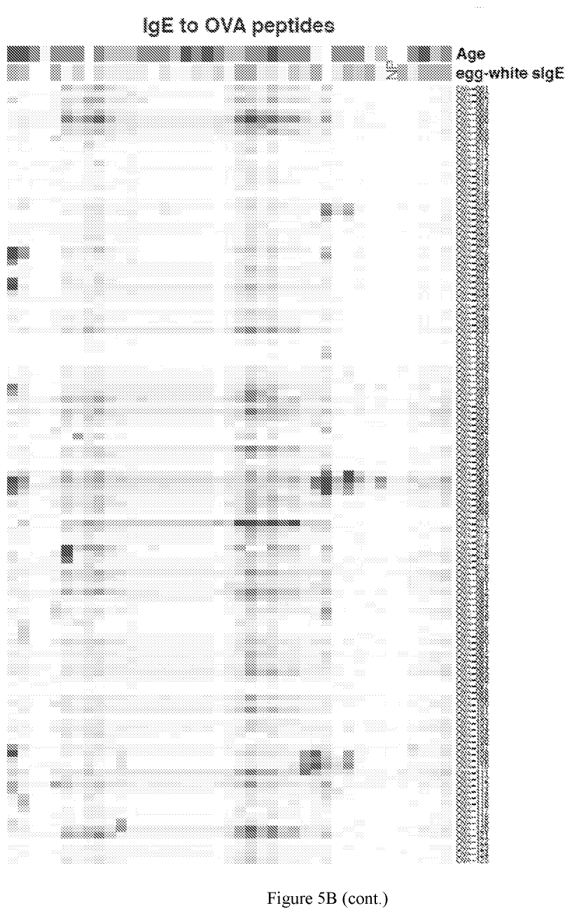

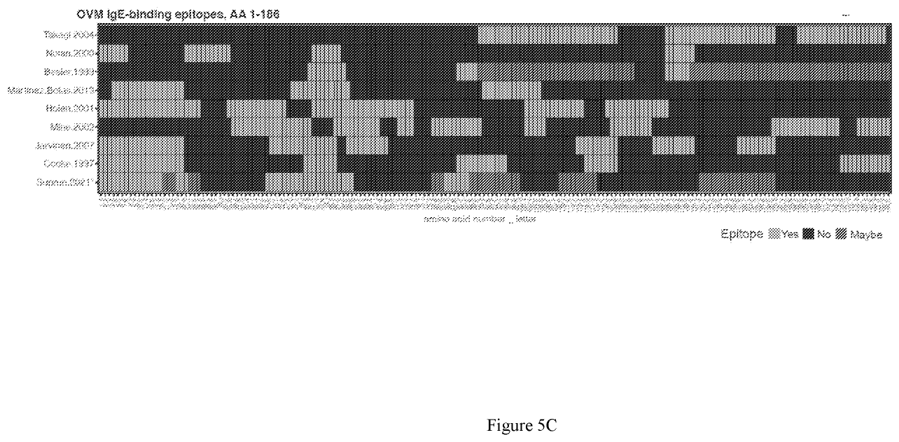

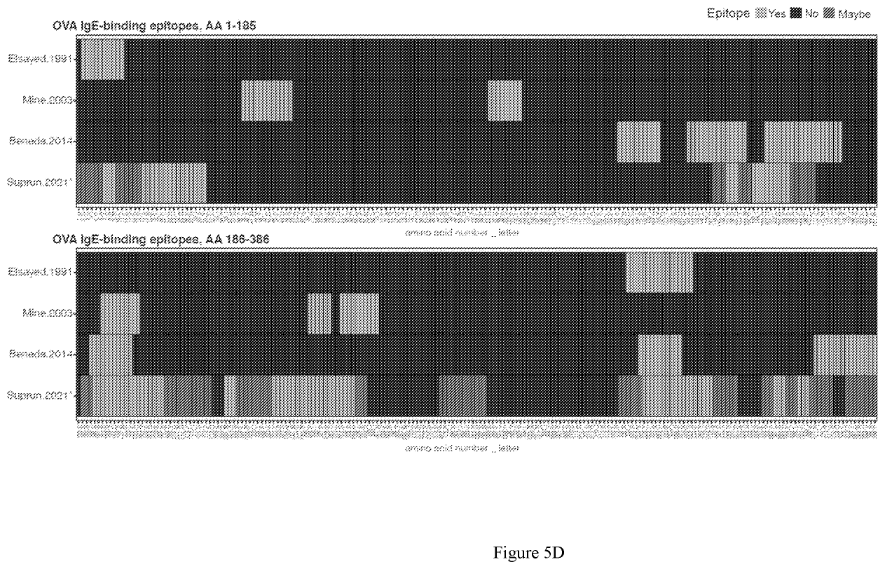

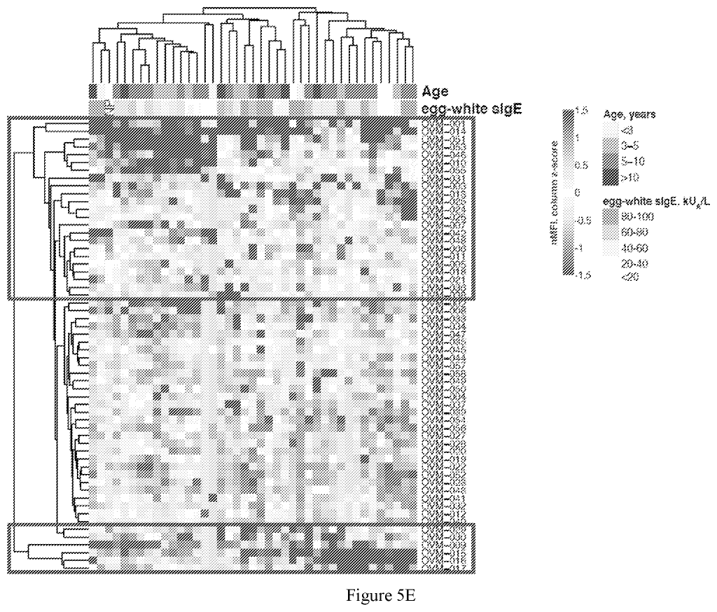

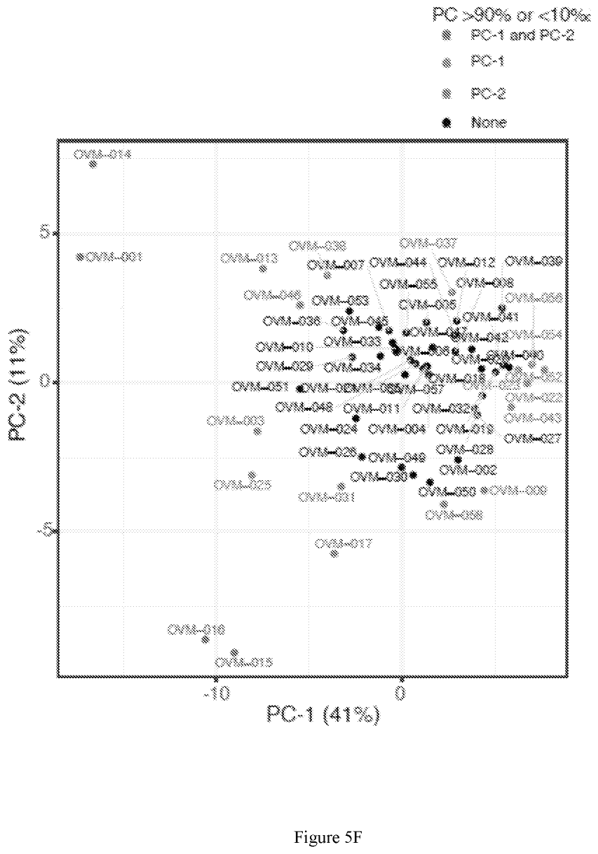

As shown in FIG. 5A, overlapping peptides covering the entire length of OVM and OVA proteins were assayed in plasma or serum of 38 egg allergic children with egg-white specific (s)IgE >0.35 kUA/L (ImmunoCAP; Uppsala, Sweden), obtained from the Food Allergy Research Initiative biorepository at Mount Sinai. Allergy status of these patients was based on sIgE or skin prick testing and/or a convincing history, unequivocal allergic reactions following oral food challenge or the ingestion of egg. A negative pool (NP) was included as a control and consisted of serum from 4 adults with no history ofany food or environmental allergies. Based on the experimental data, immunodominant peptides, i.e. epitopes, were identified (step 2).

For the third step, publicly available epitope prediction tools (see, Table 1) were used for B-cell epitope mapping for both OVM and OVA.

TABLE 1
Online epitope prediction tools

| | Prediction | | Antibody | |
|---|---|---|---|---|
| | level | Algorithm | isotype | URL |
| Algorithm name | (length) | type | specificity | (world wide web at) |
| Chou & Fasman Beta- | amino acid | structural | no | “tools.iedb.org/bcell/” |
| Turn | (varying) | | | |
| Emini Surface | amino acid | structural | no | |
| Accessibility | (varying) | | | |
| Karplus & Schulz | amino acid | structural | no | |
| Flexibility | (varying) | | | |
| Kolaskar & Tongaonkar | amino acid | structural | No | |
| Antigenicity | (varying) | | | |
| Parker Hydrophilicity | amino acid | structural | No | |
| | (varying) | | | |
| Bepipred 2.0 | peptide | ML- | No | |
| | (varying) | Random | | |
| | | Forest | | |
| BCPred 1.0 | peptide | ML-String | No | “ailab.ist.psu.edu/bcpred/” |
| | (varying) | Kernel | | |
| ABCpred | overlapping | ML- | No | “crdd.osdd.net/raghava/ |
| | peptides | Neural | | abcpred/” |
| | (16-mer) | Network | | |
| BCIgEPRED | overlapping | ML- | yes, IgE | “github.com/brsaran/ |
| | Peptides | Random | | BCIgePred” |
| | (15-mer) | Forest | | |
| CBTOPE | amino acid | ML- | No | “crdd.osdd.net/raghava/ |
| | (varying) | Support | | cbtope/” |
| | | Vector | | |
| | | Machine | | |
| Discotope v2.0 | amino acid | structural | No | “cbs.dtu.dk/services/ |
| | (varying) | | | DiscoTope/” |
| ElliPro | peptide | structural | No | “tools.iedb.org/ellipro/” |
| | (varying) | | | |

Twelve open access online B-cell epitope prediction tools listed with algorithm name; type and length of the predicted sequences (sequential peptides or individual amino acids); type of prediction (structural characteristics or machine learning (MIL) based); immunoglobulin isotype specificity; a URL link to the web server.

The predictions were then compared to the experimentally identified epitopes. In step 4, predictions from those tools were combined into a single algorithm using Random Forest to optimize the prediction accuracy. After the final algorithm was established, it was applied to predict epitopes on minor egg allergens (step 5): OVT, LYS, and sALB. Sixteen epitopes were selected for testing in the subjects described above (step 6). Additionally, 8 epitopes from YGP40 and YGP42 proteins identified based on a literature search were included (Sogawa et al., Int Arch Allergy Immunol., 2018, 176, 189-197). Protein sequences, as well as UniProt and Protein Data Bank (PDB) identifiers are provided in Table 2.

TABLE 2
Sequence and structural details for allergenic egg proteins

| | UniProt | | |
|---|---|---|---|
| Protein | ID | PDB ID | Amino Acid Sequence |
| Ovomucoid | P01005 | ModWeb_Slow_ | AEVDCSRFPNATDKEGKDVLVCNKDLR |
| (OVM; Gal d 1) | | OVM21-86.11-01- | PICGTDGVTYTNDCLLCAYSIEFGTNISK |
| | | 2018.pdb AND | EHDGECKETVPMNCSSYANTTSEDGKV |
| | | ModWeb_Slow_ | MVLCNRAFNPVCGTDGVTYDNECLLC |
| | | OVM104-210.11- | AHKVEQGASVDKRHDGGCRKELAAVS |
| | | 01-2018.pdb | VDCSEYPKPDCTAEDRPLCGSDNKTYG |
| | | | NKCNFCNAVVESNGTLTLSHFGKC |
| | | | (SEQ ID NO: 79) |
| Ovalbumin | P01012 | 1OVA chain A | MGSIGAASMEFCFDVFKELKVHHANEN |
| (OVA; Gal d 2) | | | IFYCPIAIMSALAMVYLGAKDSTRTQIN |
| | | | KVVRFDKLPGFGDSIEAQCGTSVNVHSS |
| | | | LRDILNQITKPNDVYSFSLASRLYAEER |
| | | | YPILPEYLQCVKELYRGGLEPINFQTAA |
| | | | DQARELINSWVESQTNGIIRNVLQPSSV |
| | | | DSQTAMVLVNAIVFKGLWEKAFKDEDT |
| | | | QAMPFRVTEQESKPVQMMYQIGLFRVA |
| | | | SMASEKMKILELPFASGTMSMLVLLPDE |
| | | | VSGLEQLESIINFEKLTEWTSSNVMEER |
| | | | KIKVYLPRMKMEEKYNLTSVLMAMGIT |
| | | | DVFSSSANLSGISSAESLKISQAVHAAHA |
| | | | EINEAGREVVGSAEAGVDAASVSEEFR |
| | | | ADHPFLFCIKHIATNAVLFFGRCVSP |
| | | | (SEQ ID NO: 80) |
| Ovotransferrin | P02789 | 1N04 chain A | AAPPKSVIRWCTISSPEEKKCNNLRDLT |
| (OVT; Gal d 3) | | | QQERISLTCVQKATYLDCIKAIANNEAD |
| | | | AISLDGGQAFEAGLAPYKLKPIAAEVYE |
| | | | HTEGSTTSYYAVAVVKKGTEFTVNDLQ |
| | | | GKTSCHTGLGRSAGWNIPIGTLLHRGAI |
| | | | EWEGIESGSVEQAVAKFFSASCVPGATI |
| | | | EQKLCRQCKGDPKTKCARNAPYSGYSG |
| | | | AFHCLKDGKGDVAFVKHTTVNENAPD |
| | | | QKDEYELLCLDGSRQPVDNYKTCNWA |
| | | | RVAAHAVVARDDNKVEDIWSFLSKAQS |
| | | | DFGVDTKSDFHLFGPPGKKDPVLKDLLF |
| | | | KDSAIMLKRVPSLMDSQLYLGFEYYSAI |
| | | | QSMRKDQLTPSPRENRIQWCAVGKDEK |
| | | | SKCDRWSVVSNGDVECTVVDETKDCII |
| | | | KIMKGEADAVALDGGLVYTAGVCGLV |
| | | | PVMAERYDDESQCSKTDERPASYFAVA |
| | | | VARKDSNVNWNNLKGKKSCHTAVGRT |
| | | | AGWVIPMGLIHNRTGTCNFDEYFSEGC |
| | | | APGSPPNSRLCQLCQGSGGIPPEKCVASS |
| | | | HEKYFGYTGALRCLVEKGDVAFIQHST |
| | | | VEENTGGKNKADWAKNLQMDDFELLC |
| | | | TDGRRANVMDYRECNLAEVPTHAVVV |
| | | | RPEKANKIRDLLERQEKRFGVNGSEKSK |
| | | | FMMFESQNKDLLFKDLTKCLFKVREGT |
| | | | TYKEFLGDKFYTVISSLKTCNPSDILQM |
| | | | CSFLEGK (SEQ ID NO: 81) |
| Lysozyme | P00698 | 3B6L chain A | KVFGRCELAAAMKRHGLDNYRGYSLG |
| (LYS; Gal d 4) | | | NWVCAAKFESNFNTQATNRNTDGSTD |
| | | | YGILQINSRWWCNDGRTPGSRNLCNIPC |
| | | | SALLSSDITASVNCAKKIVSDGNGMNA |
| | | | WVAWRNRCKGTDVQAWIRGCRL (SEQ |
| | | | ID NO: 82) |
| Serum albumin | P19121 | ModWeb_ | FARDAEHKSEIAHRYNDLKEETFKAVA |
| (sALB; Gal d 5) | | VerySlow_2_ | MITFAQYLQRCSYEGLSKLVKDVVDLA |
| | | sALB29- | QKCVANEDAPECSKPLPSIILDEICQVEK |
| | | 611.11-07- | LRDSYGAMADCCSKADPERNECFLSFK |
| | | 2018.pdb | VSQPDFVQPYQRPASDVICQEYQDNRV |
| | | | SFLGHFIYSVARRHPFLYAPAILSFAVDF |
| | | | EHALQSCCKESDVGACLDTKEIVMREK |
| | | | AKGVSVKQQYFCGILKQFGDRVFQARQ |
| | | | LIYLSQKYPKAPFSEVSKFVHDSIGVHK |
| | | | ECCEGDMVECMDDMARMMSNLCSQQ |
| | | | DVFSGKIKDCCEKPIVERSQCIMEAEFDE |
| | | | KPADLPSLVEKYIEDKEVCKSFEAGHDA |
| | | | FMAEFVYEYSRRHPEFSIQLIMRIAKGY |
| | | | ESLLEKCCKTDNPAECYANAQEQLNQH |
| | | | IKETQDVVKTNCDLLHDHGEADFLKSIL |
| | | | IRYTKKMPQVPTDLLLETGKKMTTIGTK |
| | | | CCQLGEDRRMACSEGYLSIVIHDTCRKQ |
| | | | ETTPINDNVSQCCSQLYANRRPCFTAMG |
| | | | VDTKYVPPPFNPDMFSFDEKLCSAPAEE |
| | | | REVGQMKLLINLIKRKPQMTEEQIKTIA |
| | | | DGFTAMVDKCCKQSDINTCFGEEGANL |
| | | | IVQSRATLGIGA (SEQ ID NO: 83) |
| YGP40 | | | AEAPSAVLENLKARCSVSYNKIKTFNEV |
| | | | KFNYSMPANCYHILVQDCSSELKFLVM |
| | | | MKSAGEATNLKAINIKIGSHEIDMHPVN |
| | | | GQVKLLVDGAESPTANISLISAGASLWI |
| | | | HNENQGFALAAPGHGIDKLYFDGKTITI |
| | | | QVPLWMAGKTCGICGKYDAECEQEYR |
| | | | MPNGYLAKNAVSFGHSWILEEAPCRGA |
| | | | CKLHRSFVKLEKTVQLAGVDSKCYSTE |
| | | | PVLRCAKGCSATKTTPVTVGFHCLPADS |
| | | | ANSLTDKQMKYDQKSEDMQDTVDAHT |
| | | | TCSCENEECST (SEQ ID NO: 84) |
| YGP42 | P87498 | | PEIASQIAQEDQSTCEVSKGDFKTFDRM |
| (Gal d 6) | | | SFTCSFNKSCNVVVAQDCTEHPKFIITTR |
| | | | KVDHQSLSREVHINTSSANITICPAADSS |
| | | | LLVTCNKESVLSDSGVSEYEKDNIKIYK |
| | | | NGKTVIVEAPIHGLKNVNFDGEILKVTV |
| | | | ASWMRGKTCGVCGNNDREKHNELLMP |
| | | | NHKLAHSCSAFVHSWVLLEETCSGGCK |
| | | | LQRRYVKLNRNPTIDGEESTCYSVDPVL |
| | | | KCMKDCTPIEKTSVKVGFHCFPKATAV |
| | | | SLLEWQRSSDKKSASEDVVESVDADID |
| | | | CTCTGDCS (SEQ ID NO: 85) |

Next (step 7), the 65 most informative epitopes were selected from all proteins (e.g., 7 egg proteins: 34 ovomucoid allergens, 19 ovalbumin allergens, 3 ovotransferrin allergens, 3 lysozyme allergens, 3 yolk serum albumin allergens, 2 YGP40 allergens, and 1 YBG42 allergen), which constituted an egg epitope library for further testing. Finally (step 8), this epitope library was used to evaluate ses-IgE and ses-IgG4 levels in egg allergic children enrolled as part of the Consortium for Food Allergy Research (Sampson et al., J. Allergy Clin. Immunol., 2019, 143, 486-493) (CoFAR7, NCT01846208) trial (Kim et al., J. Allergy Clin. Immunol., 2020, 146, 851-862 e810), atopic controls with food allergies other than egg from the CoFAR2 study (Sicherer et al., Allergy, 2019, 74, 2199-2211), and non-atopic (healthy) children with no medical or family history of food and environmental allergies, eczema, asthma and allergic rhinitis. Egg and baked-egg reactivity among CoFAR7 patients was determined using double-blind placebo-controlled oral food challenges with unheated egg-white protein (1444 mg) or muffin for baked-egg (2000 mg) (Kim et al., J. Allergy Clin. Immunol., 2020, 146, 851-862 e810).

### Measurement and Quantification of Ses-IgE and Ses-IgG4 Levels

A set of 183 overlapping peptides (15-mer, 12-mer overlap) covering the entire sequences of OVM (n=58) and OVA (n=125) proteins were commercially synthesized (CS Bio, California, USA). The Bead-Based Epitope Assay (BBEA) was carried out as described previously (Suprun et al., Sci. Rep., 2019, 9, 18425; and Suprun et al., J. Allergy Clin. Immunol., 2020, 146, 1081-1088). A master mix of peptides coupled to LumAvidin beads (Luminex Corporation, Austin, TX, USA) was prepared in PBS-TBN buffer (1×PBS, 0.02% Tween-20, 0.1% BSA) and 100 μL/well was added to 96-well filter plates. Plates were washed with PBS-TBN and 100 μL/well of 1:10 plasma dilution was incubated on a shaker for 2 hours. Then plates were incubated for 30 minutes with 50 μL/well of mouse anti-human secondary antibody (2 μg/mL of IgE-phycoerythrin (PE), clone BE5, Thermo-Fisher Scientific, Rockford, IL, USA; or 0.25 μg/mL of IgG4-PE, clone HP6025, Southern Biotech, Birmingham, AL, USA). Plates were washed and 100 μL/well of PBS-TBN was added. The signal was quantified as a Median Fluorescence Intensity (MFI) on the Luminex200 instrument (Luminex Corporation, Texas, USA). Samples were assayed in duplicates and each plate included 2 wells with only PBS-TBN, with a plate layout generated using PlateDesigner (Suprun et al., Bioinformatics, 2019, 35, 1605-1607) so that experiments are not confounded by plate and well position effects. MFIs were log 2-transformed and normalized (nMFI) by subtracting the average of the background wells using bbeaR R package v0.1.0 (Suprun et al., Sci. Rep., 2019, 9, 18425).

### OVM and OVA Epitope Mapping

Previous attempts by others to quantify epitopes on OVM and OVA using in vitro enzymatic digestion assays (Benede et al., J. Agric. Food Chem., 2014, 62, 152-158; Kovacs-Nolan et al., J. Agric. Food Chem., 2000, 48, 6261-6266; and Takagi et al., Int. Arch. Allergy Immunol., 2005, 136, 23-32), SPOT membranes (Cooke et al., J. Immunol., 1997, 159, 2026-2032; Jarvinen et al., Allergy, 2007, 62, 758-765; Mine et al., Protein Eng., 2003, 16, 747-752; and Mine et al., Biochem. Biophys. Res. Commun., 2002, 292, 1070-1074), or peptide microarrays (Martinez-Botas et al., Int. Arch. Allergy Immunol., 2013, 161, 11-20) have resulted in varying consistency across the results. A goal of the present study was to create a comprehensive egg-epitope library that can be used with BBEA technology. The peptides were synthesized with 12 amino acid overlap and an estimated binding score at individual amino acid level was calculated as a product of average nMFI and the proportion of subjects with positive signal (nMFI 2 standard deviations above the background signal). Neighboring amino acids with a score above the median of all amino acids constituted an epitope. Those epitopes were mapped to the conformational protein structure with PyMOL software (PDB 1OVA and modelled structure for OVM).

OVM and sALB's Conformational Structure Reconstruction Using Homology Modeling

Structure reconstruction of both OVM and sALB's was carried out using homology modeling via ModBase server (world wide web at “modbase.compbio.ucsf.edu/”) with ModPipe (Pieper et al., Nucleic Acids Res., 2011, 39, D465-474). Structure quality was assessed by Structural Analysis and Verification Server v5.0 (world wide web at “servicesn.mbi.ucla.edu/SAVES/”) using ERRAT, PROCHECK, WHATCEHCK, and Verify 3D metrics (Eisenberg et al., Methods Enzymol., 1997, 277, 396-404; Wiederstein et al., Nucleic Acids Res., 2007, 35, W407-410; Bowie et al., Science, 1991, 253, 164-170; Colovos et al., Protein Sci., 1993, 2, 1511-1519; Hooft et al., Nature, 1996, 381, 272; and Luthy et al., Nature, 1992, 356, 83-85). The final OVM's structure consisted of two models for the positions at amino acids 21-86 (template from Sus scrofa's kazal inhibitor, PDB 1PCE, 38% sequence identity) and amino acids 104-210 (template from insect derived kazal inhibitor, PDB 1TBR, 35% sequence identity). sALB was modeled using the crystal structure of bovine serum albumin (PDB 4F5S, 44% sequence identity) as a template, with successful reconstruction of almost the whole protein (amino acids 29-611).

### Computational Epitope Predictions

A predictor based on an ensemble of 12 B-cell epitope prediction tools was developed (see, Table 1): BCIgEPred (Saravanan et al., Mol. Biol. (Mosk)., 2018, 52, 333-343), CBTOPE (Ansari et al., Immunome Res., 2010, 6, 6), BCPred (El-Manzalawy et al., J. Mol. Recognit., 2008, 21, 243-255), ElliPro (Ponomarenko et al., BMC Bioinformatics, 2008, 9, 514), DiscoTope (Kringelum et al., PLoS Comput. Biol., 2012, 8, e1002829), ABCpred (Saha et al., Proteins, 2006, 65, 40-48), BepiPred 2.0 (Jespersen et al., Nucleic Acids Res., 2017, 45, W24-W29), and IEDB resources (world wide web at “tools.iedb.org/main/bcell/”): Karplus-Schulz Flexibility, Parker Hydrophilicity (Parker et al., Biochemistry, 1986, 25, 5425-5432), Emini Surface Accessibility (Emini et al., J. Virol., 1985, 55, 836-839), Chou-Fasman Beta-Turn (Chou et al., Adv. Enzymol. Relat. Areas Mol. Biol., 1978, 47, 45-148), Kolaskar-Tongaonkar Antigenicity (Kolaskar et al., FEBS Lett., 1990, 276, 172-174). The prediction consisted of the following steps:
i) All 12 tools with their default settings were used to obtain amino acid-level predictions using an entire OVM and half of the OVA sequence for training, and the rest of the OVA for testing.
ii) Random Forest algorithm (caret package v6.0) identified the best combination of those algorithms that predicted experimentally discovered OVM and OVA epitopes, and
iii) This ensemble algorithm was used to detect epitopes on minor proteins, of which the top 5 OVT, 6 LYS, and 5 sALB candidates were synthesized. Additionally, 3 peptides for YGP42 protein were selected based on IEDB resources (due to the lack of structural templates); and for a newly identified allergen—YGP40—5 epitopes were included (Sogawa et al., Int. Arch. Allergy Immunol., 2018, 176, 189-197).
iv) These peptides (see, Table 3) were evaluated for IgE binding from the same subjects used for the OVM and OVA epitope mapping.

TABLE 3
Amino acid sequences of 24 top epitope
candidates synthesized for testing with BBEA

| Protein | Sequence |
|---|---|
| OVT_1 | SSPEEKKCNNLRDLT (SEQ ID NO: 60) |
| OVT_2 | LRDLTQQERISLTCV (SEQ ID NO: 61) |
| OVT_3 | TIEQKLCRQCKGDPK (SEQ ID NO: 62) |
| OVT_4 | DQLTPSPRENRIQWC (SEQ ID NO: 63) |
| OVT_5 | VMDYRECNLAEVPTH (SEQ ID NO: 64) |
| LYS_1 | AAAMKRHGLDNYRGY (SEQ ID NO: 54) |
| LYS_2 | GYSLGNWVCAAKFES (SEQ ID NO: 55) |
| LYS_3 | RNTDGSTDYGILQIN (SEQ ID NO: 56) |
| LYS_4 | TDYGILQINSRWWCN (SEQ ID NO: 57) |
| LYS_5 | WCNDGRTPGSRNLCN (SEQ ID NO: 58) |
| LYS_6 | WVAWRNRCKGTDVQA (SEQ ID NO: 59) |
| sALB_1 | YQRPASDVICQEYQD (SEQ ID NO: 65) |
| sALB_2 | LEKCCKTDNPAECYA (SEQ ID NO: 67) |
| sALB_3 | KQETTPINDNVSQCC (SEQ ID NO: 68) |
| sALB_4 | GVDTKYVPPPFNPDM (SEQ ID NO: 69) |
| sALB_5 | MVDKCCKQSDINTCFGE (SEQ ID NO: 70) |
| YGP42_1 | VDHQSLSREVHINTS (SEQ ID NO: 76) |
| YGP42_2 | GVCGNNDREKHNELL (SEQ ID NO: 77) |
| YGP42_3 | SSDKKSASEDVVESV (SEQ ID NO: 78) |
| YGP40_1 | NYSMPANCYHILVQD (SEQ ID NO: 71) |
| YGP40_2 | VQDCSSELKFLVMMK (SEQ ID NO: 72) |
| YGP40_3 | CAKGCSATKTTPVTV (SEQ ID NO: 73) |
| YGP40_4 | CSATKTTPVTVGFHC (SEQ ID NO: 74) |
| YGP40_5 | FHCLPADSANSLTDK (SEQ ID NO: 75) |

### Egg Epitope Library

Several descriptive analyses were used to identify informative epitopes from OVM and OVA proteins, including unsupervised hierarchical clustering, with peptides having higher nMFI values grouped together. Epitopes with high variability across patients could be helpful when studying different allergy phenotypes. To assure such epitopes were included, additional selection kept peptides with a coefficient of variation (CV) >90% or extreme values (below 10th or above 90th percentile) of the 1 st or 2nd components of the principal component analysis (PCA). The final library consisted of 65 epitopes (see, Table 4).

TABLE 4
Amino acid sequences of 65 epitopes
selected for the egg epitope library

| Peptide | Protein | Sequence |
|---|---|---|
| OVM-001 | Ovomucoid | AEVDCSRFPNATDKE (SEQ ID NO: 1) |
| OVM-003 | Ovomucoid | RFPNATDKEGKDVLV (SEQ ID NO: 2) |
| OVM-004 | Ovomucoid | NATDKEGKDVLVCNK (SEQ ID NO: 3) |
| OVM-005 | Ovomucoid | DKEGKDVLVCNKDLR (SEQ ID NO: 4) |
| OVM-007 | Ovomucoid | VLVCNKDLRPICGTD (SEQ ID NO: 5) |
| OVM-008 | Ovomucoid | CNKDLRPICGTDGVT (SEQ ID NO: 6) |
| OVM-009 | Ovomucoid | DLRPICGTDGVTYTN (SEQ ID NO: 7) |
| OVM-010 | Ovomucoid | PICGTDGVTYTNDCL (SEQ ID NO: 8) |
| OVM-013 | Ovomucoid | YTNDCLLCAYSIEFG (SEQ ID NO: 9) |
| OVM-014 | Ovomucoid | DCLLCAYSIEFGTNI (SEQ ID NO: 10) |
| OVM-015 | Ovomucoid | LCAYSIEFGTNISKE (SEQ ID NO: 11) |
| OVM-018 | Ovomucoid | TNISKEHDGECKETV (SEQ ID NO: 12) |
| OVM-021 | Ovomucoid | ECKETVPMNCSSYAN (SEQ ID NO: 13) |
| OVM-023 | Ovomucoid | PMNCSSYANTTSEDG (SEQ ID NO: 14) |
| OVM-025 | Ovomucoid | YANTTSEDGKVMVLC (SEQ ID NO: 15) |
| OVM-027 | Ovomucoid | EDGKVMVLCNRAFNP (SEQ ID NO: 16) |
| OVM-028 | Ovomucoid | KVMVLCNRAFNPVCG (SEQ ID NO: 17) |
| OVM-031 | Ovomucoid | FNPVCGTDGVTYDNE (SEQ ID NO: 18) |
| OVM-032 | Ovomucoid | VCGTDGVTYDNECLL (SEQ ID NO: 19) |
| OVM-033 | Ovomucoid | TDGVTYDNECLLCAH (SEQ ID NO: 20) |
| OVM-036 | Ovomucoid | CLLCAHKVEQGASVD (SEQ ID NO: 21) |
| OVM-038 | Ovomucoid | KVEQGASVDKRHDGG (SEQ ID NO: 22) |
| OVM-040 | Ovomucoid | SVDKRHDGGCRKELA (SEQ ID NO: 23) |
| OVM-041 | Ovomucoid | KRHDGGCRKELAAVS (SEQ ID NO: 24) |
| OVM-042 | Ovomucoid | DGGCRKELAAVSVDC (SEQ ID NO: 25) |
| OVM-044 | Ovomucoid | ELAAVSVDCSEYPKP (SEQ ID NO: 26) |
| OVM-046 | Ovomucoid | VDCSEYPKPDCTAED (SEQ ID NO: 27) |
| OVM-048 | Ovomucoid | PKPDCTAEDRPLCGS (SEQ ID NO: 28) |
| OVM-050 | Ovomucoid | AEDRPLCGSDNKTYG (SEQ ID NO: 29) |
| OVM-052 | Ovomucoid | CGSDNKTYGNKCNFC (SEQ ID NO: 30) |
| OVM-054 | Ovomucoid | TYGNKCNFCNAVVES (SEQ ID NO: 31) |
| OVM-055 | Ovomucoid | NKCNFCNAVVESNGT (SEQ ID NO: 32) |
| OVM-056 | Ovomucoid | NFCNAVVESNGTLTL (SEQ ID NO: 33) |
| OVM-058 | Ovomucoid | VESNGTLTLSHFGKC (SEQ ID NO: 34) |
| OVA-001 | Ovalbumin | MGSIGAASMEFCFDV (SEQ ID NO: 35) |
| OVA-003 | Ovalbumin | ASMEFCFDVFKELKV (SEQ ID NO: 36) |
| OVA-006 | Ovalbumin | FKELKVHHANENIFY (SEQ ID NO: 37) |
| OVA-021 | Ovalbumin | DKLPGFGDSIEAQCG (SEQ ID NO: 38) |
| OVA-028 | Ovalbumin | SSLRDILNQITKPND (SEQ ID NO: 39) |
| OVA-040 | Ovalbumin | YLQCVKELYRGGLEP (SEQ ID NO: 40) |
| OVA-053 | Ovalbumin | IIRNVLQPSSVDSQT (SEQ ID NO: 41) |
| OVA-057 | Ovalbumin | SQTAXVLVNAIVFKG (SEQ ID NO: 42) |
| OVA-059 | Ovalbumin | LVNAIVFKGLWEKAF (SEQ ID NO: 43) |
| OVA-064 | Ovalbumin | KDEDTQAMPFRVTEQ (SEQ ID NO: 44) |
| OVA-068 | Ovalbumin | TEQESKPVQMMYQIG (SEQ ID NO: 45) |
| OVA-071 | Ovalbumin | MMYQIGLFRVASMAS (SEQ ID NO: 46) |
| OVA-075 | Ovalbumin | MASEKMKILELPFAS (SEQ ID NO: 47) |
| OVA-085 | Ovalbumin | LEQLESIINFEKLTE (SEQ ID NO: 48) |
| OVA-090 | Ovalbumin | WTSSNVMEERKIKVY (SEQ ID NO: 49) |
| OVA-095 | Ovalbumin | LPRMKMEEKYNLTSV (SEQ ID NO: 50) |
| OVA-108 | Ovalbumin | LKISQAVHAAHAEIN (SEQ ID NO: 51) |
| OVA-113 | Ovalbumin | EAGREVVGSAEAGVD (SEQ ID NO: 52) |
| OVA-120 | Ovalbumin | EFRADHPFLFCIKHI (SEQ ID NO: 53) |
| OVT_1 | Ovotrans- | SSPEEKKCNNLRDLT (SEQ ID NO: 60) |
| | ferrin | |
| OVT_3 | Ovotrans- | TIEQKLCRQCKGDPK (SEQ ID NO: 62) |
| | ferrin | |
| OVT_5 | Ovotrans- | VMDYRECNLAEVPTH (SEQ ID NO: 64) |
| | ferrin | |
| LYS_1 | Lysozyme | AAAMKRHGLDNYRGY (SEQ ID NO: 54) |
| LYS_3 | Lysozyme | RNTDGSTDYGILQIN (SEQ ID NO: 56) |
| LYS_6 | Lysozyme | WVAWRNRCKGTDVQA (SEQ ID NO: 59) |
| sALB_1 | Serum | LRDSYGAMADCCSKA (SEQ ID NO: 66) |
| | albumin | |
| sALB_4 | Serum | KQETTPINDNVSQCC (SEQ ID NO: 68) |
| | albumin | |
| sALB_5 | Serum | GVDTKYVPPPFNPDM (SEQ ID NO: 69) |
| | albumin | |
| YGP40_1 | YGP40 | NYSMPANCYHILVQD (SEQ ID NO: 71) |
| YGP40_5 | YGP40 | FHCLPADSANSLTDK (SEQ ID NO: 75) |
| YGP42_1 | YGP42 | VDHQSLSREVHINTS (SEQ ID NO: 76) |

### Statistical Analyses

Statistical analyses were performed in R v3.5. Technical duplicates for each sample and epitope were averaged. Group comparisons were presented as ANOVA p-values obtained by fitting a linear regression. Differences in sequential ses-IgE and ses-IgG4 between egg allergic, atopic, and healthy groups were assessed by age-adjusted linear models, using the empirical Bayes method in the limma package v3.42, allowing estimation of the variance parameters across all epitopes (Ritchie et al., Nucleic Acids Res., 2015, 43, e47; and Smyth, Stat. Appl. Genet. Mol. Biol., 2004, 3, Article3). The results are presented as estimated marginal means (EMmean) for each group; p-values were adjusted for multiple testing using the Benjamini-Hochberg approach, controlling the False Discovery Rate (FDR, <0.05). For each patient, the overall IgE and IgG4 antibody levels of either all 65 epitopes or 17 epitopes with significant differences in BER vs BET comparison were summarized as z-scores.

### Example 2: Epitope Mapping of OVM and OVA Proteins

Fifty-eight OVM and 126 OVA peptides were evaluated for IgE binding using BBEA in 38 egg allergic subjects with median egg white sIgE of 50 kUA/L and 1 control pool (NP, see, FIG. 5B). The average age of allergic patients was 6.6 years; 58% were male, mostly of white race (75%), with more than 50% having atopic comorbidities, such as atopic dermatitis, asthma, and/or allergic rhinitis (see, Table 5).

TABLE 5
Demographic and clinical characteristics of 38 egg allergic subjects

| | Egg Allergic |
|---|---|
| | (n = 38) |
| Age, mean (SD) | 6.6 (3.6) |
| Male, n (%) | 22 (57.9) |
| Race, n (%) | |
| Asian | 5 (14.7) |
| Other | 2 (5.9) |
| White/Caucasian | 27 (79.4) |
| Not Hispanic or Latino, n (%) | 30 (93.8) |
| Asthma, n (%) | 28 (73.7) |
| Rhinitis, n (%) | 22 (57.9) |
| Eczema, n (%) | 29 (76.3) |
| Egg-white sIgE (kU_{A}/L), median [Q1-Q3] | 49.9 (22.8-90.5) |

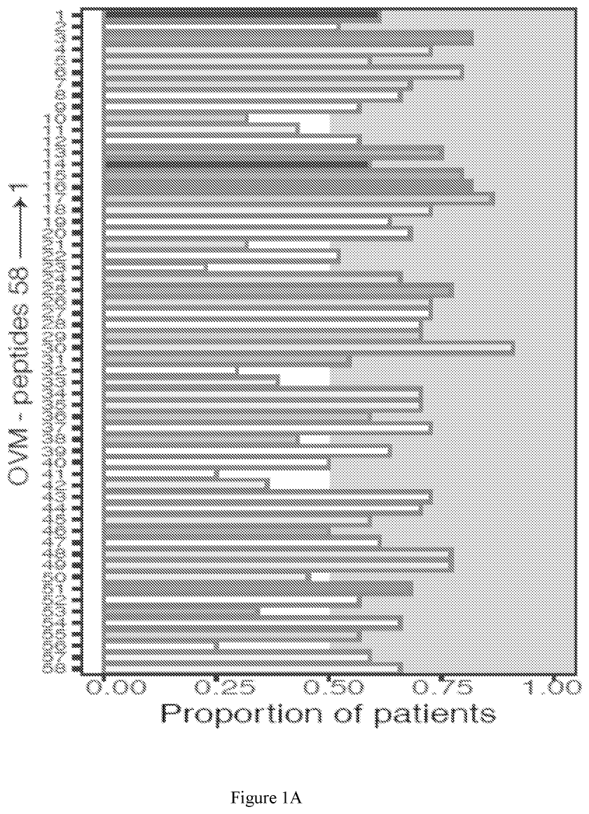

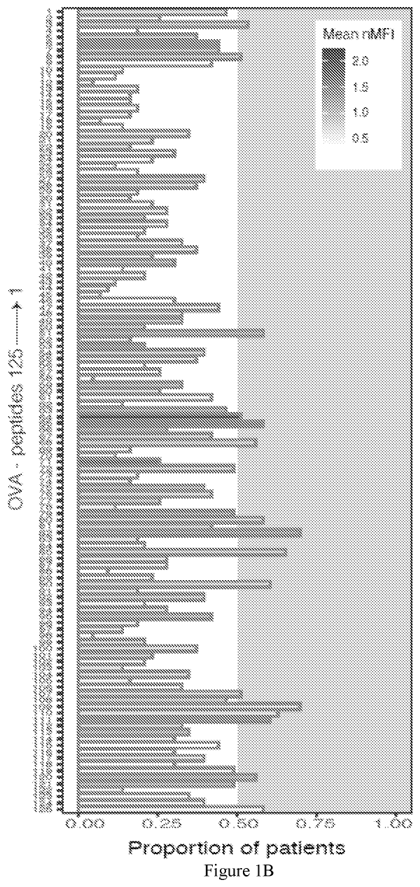

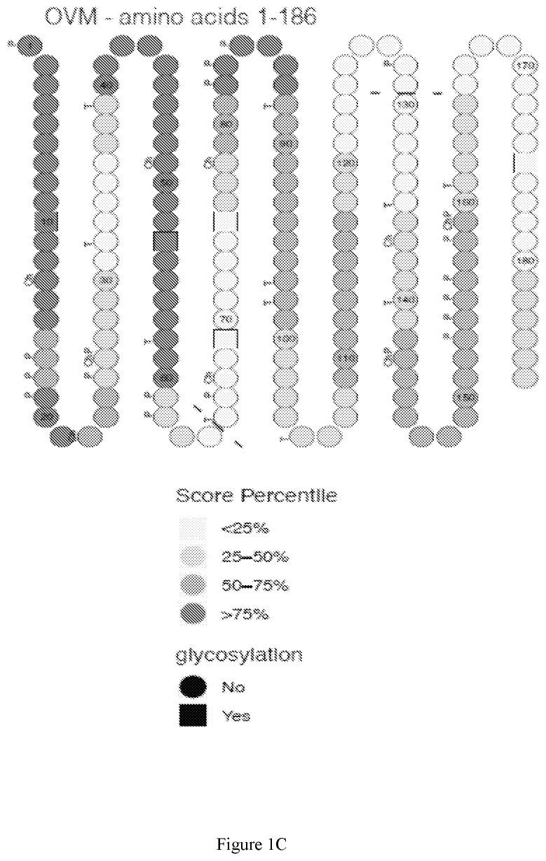

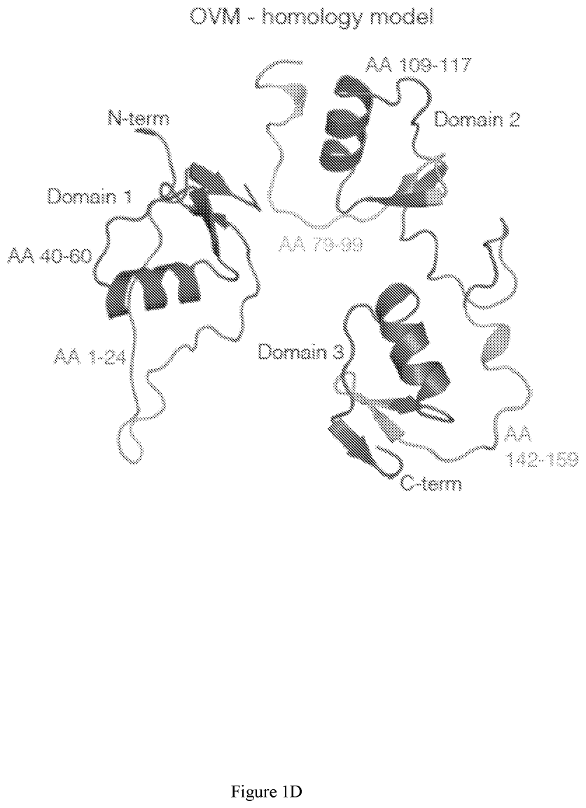

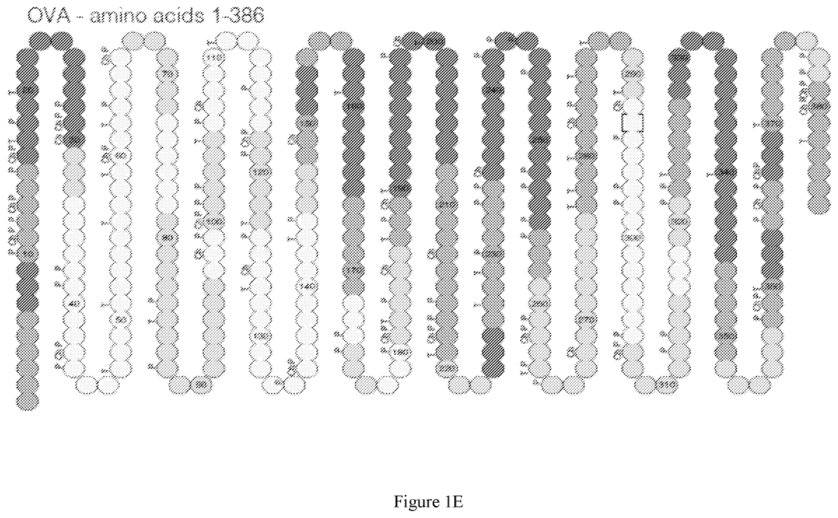

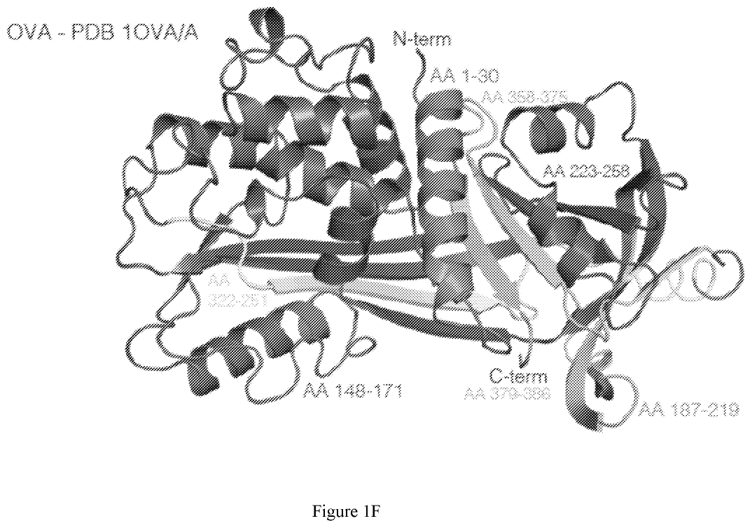

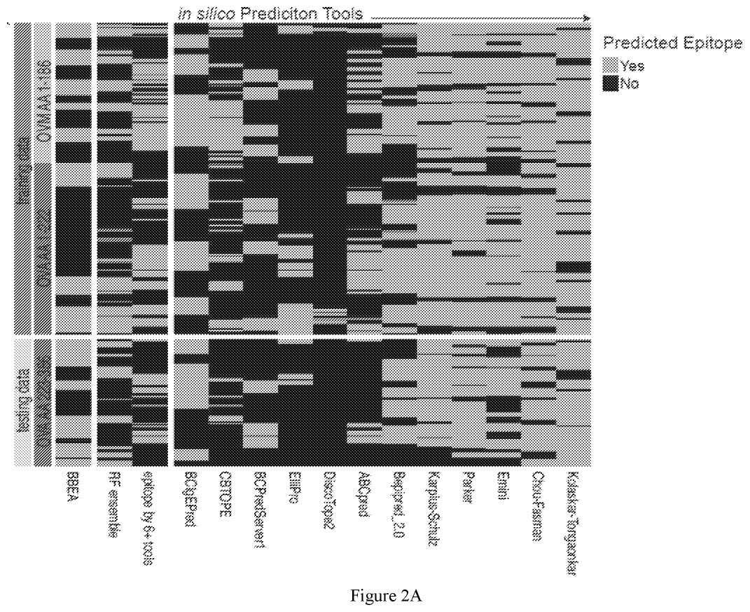

SD = standard deviation;
Q1 = 1^{st} quartile;
Q3 = 3^{rd} quartile
ses-IgE bound to a greater number of OVM peptides and was found in more patients compared to ses-IgE to OVA: 46/58 (79%) of OVM peptides were detected in at least 50% of patients, while this was the case for only 14/126 (11%) of OVA peptides (see, FIG. 1A and FIG. 1B).

The nMFI values for the overlapping peptides were collapsed to the AA-level to identify immunodominant epitopes. Such epitopes were mapped to all three domains of OVM, with higher scores at the N-terminus (1st domain) of the protein (see, FIG. 1C), and in several regions of the OVA (see, FIG. 1E). When overlayed to the conformational structure, epitopes were located on the surface as well as buried in parts of the proteins (see, FIG. 1D and FIG. 1F). This indicates that protein breakdown or conformational change had to occur for the IgE to bind some of those residues. OVM epitopes identified with BBEA have been described in at least 2 out of 8 previous studies, while several novel IgE-binding regions were mapped for OVA (see, FIG. 5C and FIG. 5D).

### Example 3: In Silico Prediction of Epitopes on Minor Egg Allergens

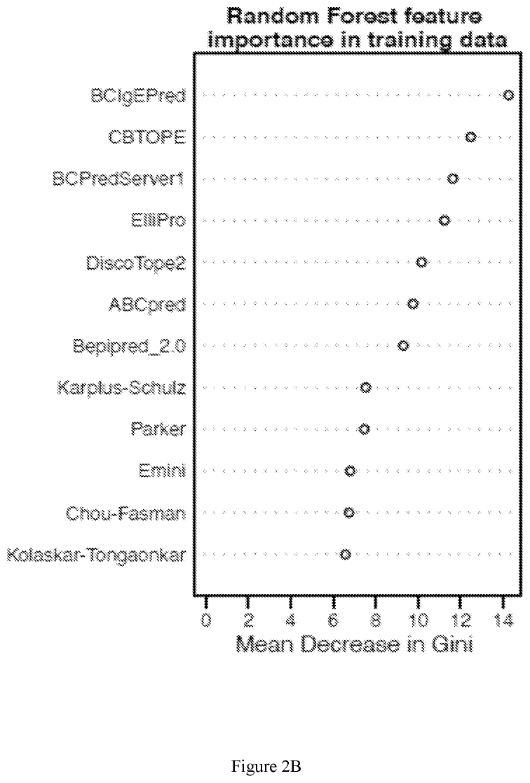

Using a combination of 12 B-cell epitope prediction tools, epitopes were first predicted on OVM and OVA proteins. Those epitopes were then compared to the ones mapped experimentally (see, FIG. 2A). A variability of predictions made it difficult to commit to a single algorithm (median positive predictive value (PPV)=0.6). The experimental data was used to build an ensemble that optimally combined all tools into a ‘crowd prediction’ using a Random Forest algorithm. The resultant model was tested on the remainder of the OVA protein (see, FIG. 2A bottom) and showed some predictive improvement (PPV=0.7). This new algorithm was then applied to identify epitopes of minor allergens: OVT, LYS, and sALB. Sixteen epitopes from three minor allergens, and 8 from YGP42 and YGP40 were synthesized (see, Table 3).

While all 12 tools contributed to the predictions, their contributions were weighted differently by the Random Forest with BCIgEPred (Saravanan et al., Mol. Biol. (Mosk), 2018, 52, 333-343) having the highest importance index (see, FIG. 2B).

### Example 4: Experimental Testing of Epitopes from Minor Allergens and Egg Epitope Library

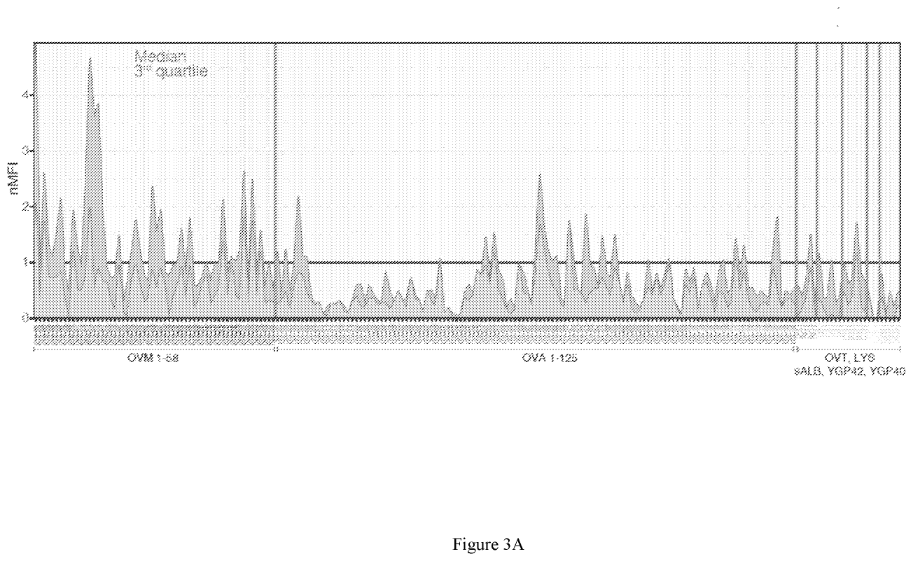

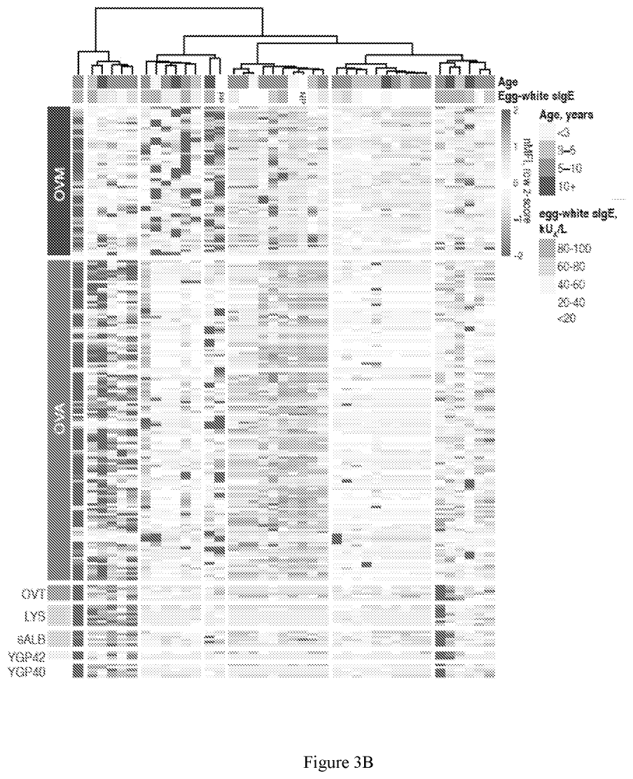

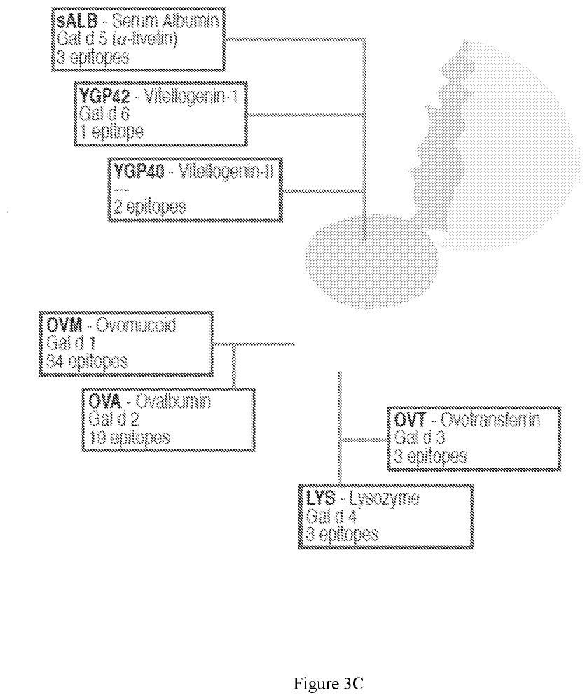

The 24 potential epitopes from OVT, LYS, sALB, YGP42, and YGP40 were evaluated for IgE binding using the BBEA in the same patient cohort that was used for OVM and OVA epitope mapping. As shown in FIG. 3A, OVM and OVA peptides had the highest median nMFI, while IgE against minor allergens generally had lower levels. However, in a small number of patients (8/38) those IgE antibodies did show different profiles: compared to the rest of the patients, their IgE bound epitopes on OVA and minor allergens (see, FIG. 3B, right cluster).

To create a comprehensive egg epitope library, epitopes from all proteins were selected with more epitopes chosen from the major allergens (see, FIG. 3C). The selection was based on both signal intensity and variability, to make sure a diverse set of epitopes was present (see, FIG. 5E and FIG. 5F). A few peptides with the most IgE binding from the minor proteins were also included, since it was not desired to rule out the possibility that they could still be informative in a larger patient cohort with different levels of egg allergy severity or phenotype. The final library consisted of 65 epitopes from 7 egg proteins (see, Table 4).

### Example 5: Ses-IgE is Associated with Baked Egg Reactivity

The 65-plex epitope library was evaluated on anew pediatric cohort for both IgE and IgG4 reactivity. The new cohort included 135 egg allergic patients, of whom 82 individuals were reactive to baked-egg (BER), 53 could tolerate baked-egg products (BET), 46 had atopic comorbidities or food allergies other than egg, and 11 were healthy subjects (see, Table 6).

TABLE 6
Demographic and clinical characteristics of egg allergic children, atopic and healthy controls

| | BER | BET | Atopic | Healthy |
|---|---|---|---|---|
| | n = 82 | n = 53 | n = 46 | n = 11 |
| Age, years (mean, SD) | 8.4 (2.5) | 8.3 (3.2) | 6.6 (2.7) | 9.9 (4.2) |
| Male sex (n, %) | 44 (53.7) | 34 (64.2) | 28 (60.9) | 7 (63.6) |
| Race (n, %) | | | | |
| Asian | 7 (8.5) | 4 (7.5) | 6 (13.6) | 0 (0.0) |
| Black/African American | 2 (2.4) | 3 (5.7) | 4 (9.1) | 2 (66.7) |
| White | 63 (76.8) | 43 (81.1) | 33 (75.0) | 1 (33.3) |
| Other | 10 (12.2) | 3 (5.7) | 1 (2.3) | 0 (0.0) |
| Asthma history (n, %) | 50 (61.0) | 32 (60.4) | 21 (47.7) | 0 (0.0) |
| Allergic rhinitis history (n, %) | 68 (82.9) | 45 (84.9) | 26 (59.1) | 0 (0.0) |
| Atopic dermatitis history (n, %) | 52 (63.4) | 34 (64.2) | 24 (54.5) | 0 (0.0) |
| Baked egg OFC, mg | 550 | 2000 | • | • |
| (median, Q1-Q3) | [100-780] | [2000-2000] | | |
| Egg-white OFC, mg | • | 144 | • | • |
| (median, Q1-Q3) | | [44-444] | | |
| Serology (median, Q1-Q3) | | | | |
| Total IgE, kU/L | 862 | 873 | 357 | 74.8 |
| | [392-1677] | [304-1741] | [151-1001] | [28.9-98.5] |
| Egg IgE, kU_{A}/L | 24.2 | 10.8 | 0.1 | |
| | [12.7-65.0] | [6.7-27.1] | [0.1-0.5] | |
| Ovalbumin IgE, kU_{A}/L | 14.7 | 5.8 | • | • |
| | [6.0-29.6] | [3.4-17.4] | | |
| Ovomucoid IgE, KU_{A}/L | 15.1 | 7.0 | • | • |
| | [6.1-38.1] | [2.5-13.4] | | |
| Egg IgG, mg/mL | 5.2 | 7.5 | • | • |
| | [2.9-8.5] | [3.8-13.1] | | |
| Egg IgG₄, mg/mL | 0.5 | 0.8 | • | • |
| | [0.2-1.1] | [0.2-3.1] | | |
| Ovalbumin IgG₄, mg/mL | 0.4 | 0.6 | • | • |
| | [0.1-1.0] | [0.1-1.9] | | |
| Ovomucoid IgG₄, mg/mL | 0.1 | 0.2 | • | • |
| | [0.0-0.3] | [0.0-1.4] | | |
| Z-scores of ses-IgE (mean, SD) | | | | |
| all 65 epitopes | 2.0 (7.9) | −0.7 (2.4) | −2.1 (1.7) | −1.9 (1.7) |
| ovomucoid's epitopes | 1.6 (5.2) | −0.3 (2.3) | −1.7 (1.4) | −1.6 (1.3) |
| ovalbumin's epitopes | 1.0 (4.7) | −0.6 (1.1) | −1.0 (0.9) | −0.9 (0.9) |
| egg-white's epitopes | 2.0 (7.3) | −0.7 (2.4) | −2.0 (1.7) | −1.9 (1.6) |
| egg yolk's epitopes | 0.6(3.1) | −0.3 (0.8) | −0.6 (0.7) | −0.4 (0.5) |
| Z-scores of ses-IgG₄ (mean, SD) | | | | |
| all 65 epitopes | −3.9 (6.0) | −4.8 (4.1) | −1.7 (5.8) | −3.4 (3.6) |
| ovomucoid's epitopes | −3.0 (4.4) | −3.7 (3.3) | −1.2 (4.3) | −2.4 (2.8) |
| ovalbumin's epitopes | −1.9 (3.1) | −2.4 (2.0) | −1.0 (3.1) | −1.9 (1.8) |
| egg-white's epitopes | −3.7 (5.7) | −4.6 (3.9) | −1.6 (5.5) | −3.2 (3.4) |
| egg yolk's epitopes | −1.2 (1.9) | −1.4 (1.2) | −0.6 (1.8) | −1.0 (1.1) |

| | p-value | p-value |
|---|---|---|
| | (all groups) ξ | (BER vs BET) ξξ |
| Age, years (mean, SD) | 0.001 | 0.796 |
| Male sex (n, %) | 0.635 | 0.304 |
| Race (n, %) | 0.001 | 0.485 |
| Asian | | |
| Black/African American | | |
| White | | |
| Other | | |
| Asthma history (n, %) | 0.099 | 0.999 |
| Allergic rhinitis history (n, %) | <0.001 | 0.948 |
| Atopic dermatitis history (n, %) | 0.001 | 0.999 |
| Baked egg OFC, mg | • | <0.001 |
| (median, Q1-Q3) | | |
| Egg-white OFC, mg | • | • |
| (median, Q1-Q3) | | |
| Serology (median, Q1-Q3) | | |
| Total IgE, kU/L | <0.001 | 0.968 |
| Egg IgE, KU_{A}/L | • | <0.001 |
| Ovalbumin IgE, KU_{A}/L | • | 0.001 |
| Ovomucoid IgE, KU_{A}/L | • | 0.001 |
| Egg IgG, mg/mL | • | 0.027 |
| Egg IgG₄, mg/mL | • | 0.044 |
| Ovalbumin IgG₄, mg/mL | • | 0.134 |
| Ovomucoid IgG₄, mg/mL | • | 0.091 |
| Z-scores of ses-IgE (mean, SD) | | |
| all 65 epitopes | <0.001 | 0.014 |
| ovomucoid's epitopes | <0.001 | 0.013 |
| ovalbumin's epitopes | 0.002 | 0.016 |
| egg-white's epitopes | <0.001 | 0.012 |
| egg yolk's epitopes | 0.013 | 0.051 |
| Z-scores of ses-IgG₄ (mean, SD) | | |
| all 65 epitopes | 0.04 | 0.346 |
| ovomucoid's epitopes | 0.02 | 0.355 |
| ovalbumin's epitopes | 0.087 | 0.3 |
| egg-white's epitopes | 0.037 | 0.339 |
| egg yolk's epitopes | 0.091 | 0.431 |

SD = standard deviation;
Q1 = 1st quartile;
Q3 = 3rd quartile.
ξ = ANOVA p-values for the difference among 4 groups obtained by fitting a simple linear regression model.
ξξ = P-values for the comparison of the BER and BET groups using t-test for normally distributed variables, Wilcoxon-Mann-Whitney test for non-normal variables, and chi-squared test for categorical variables.

The healthy control group appeared slightly older compared to the atopic and egg allergic children, with median ages of 10 vs 6 vs 8 years, respectively. To account for this when comparing the ses-IgE and ses-IgG4, the models were adjusted for age.

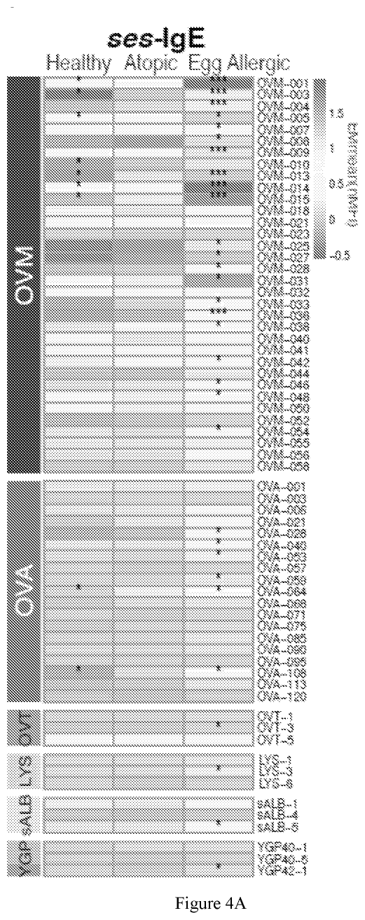

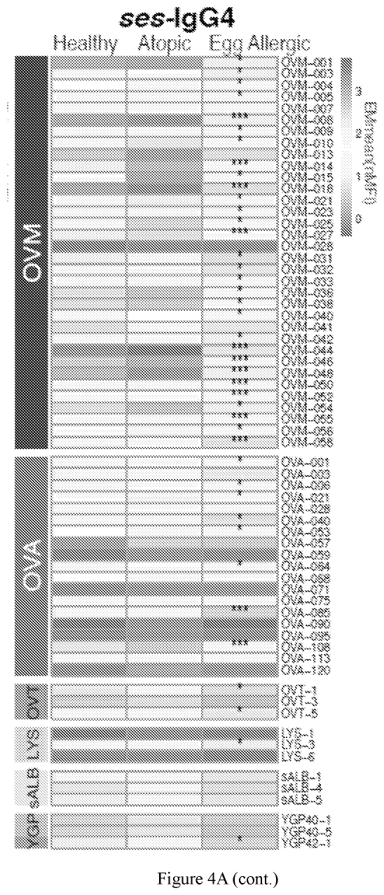

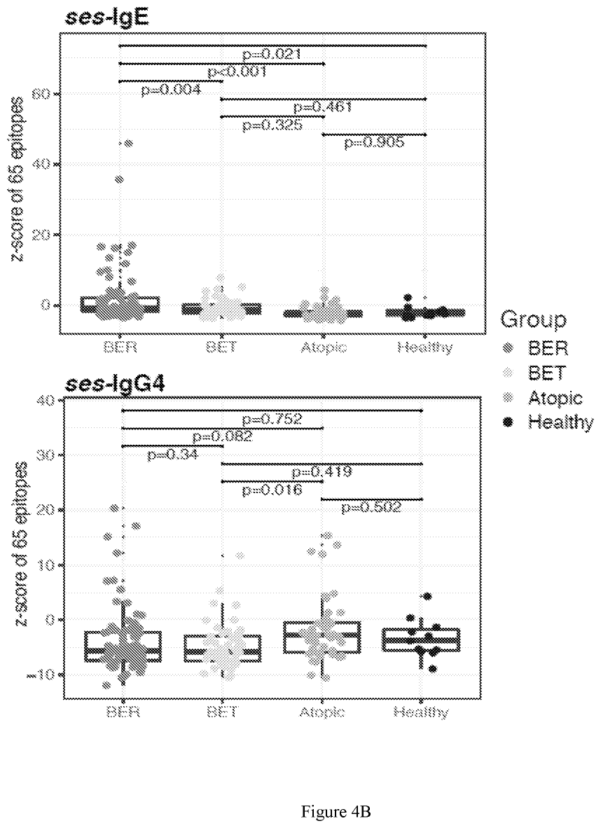

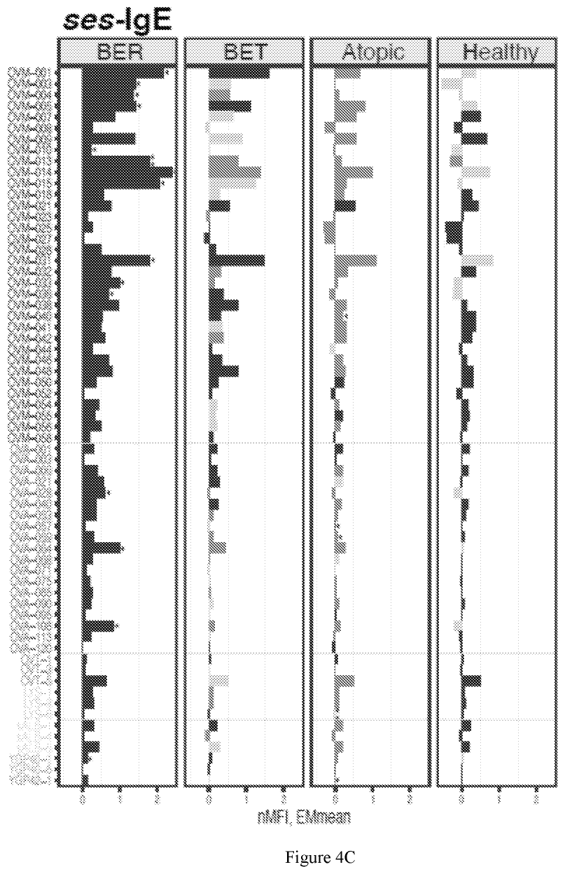

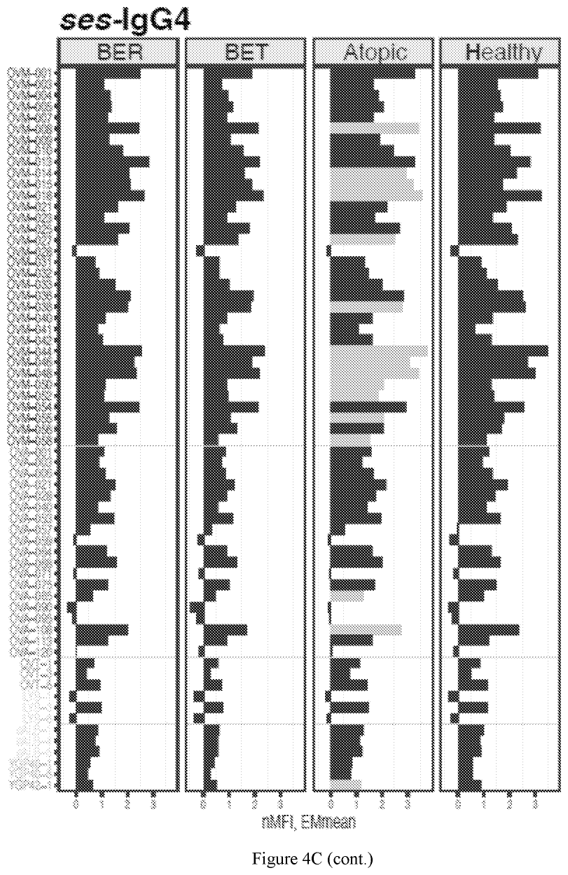

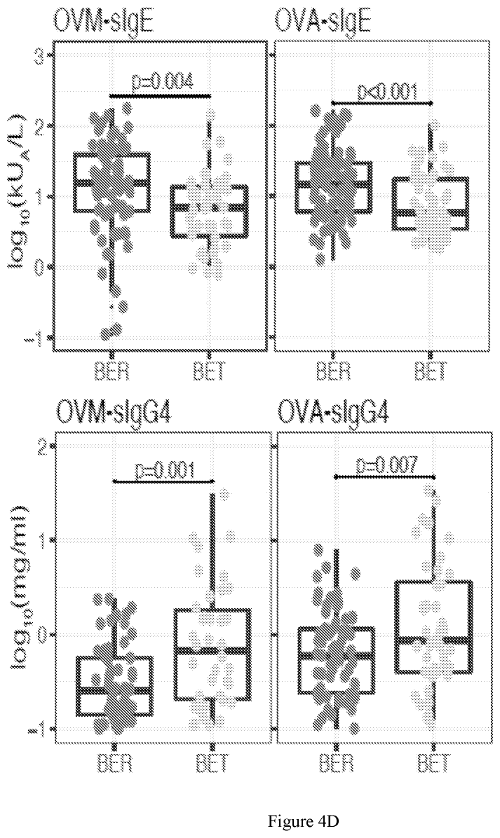

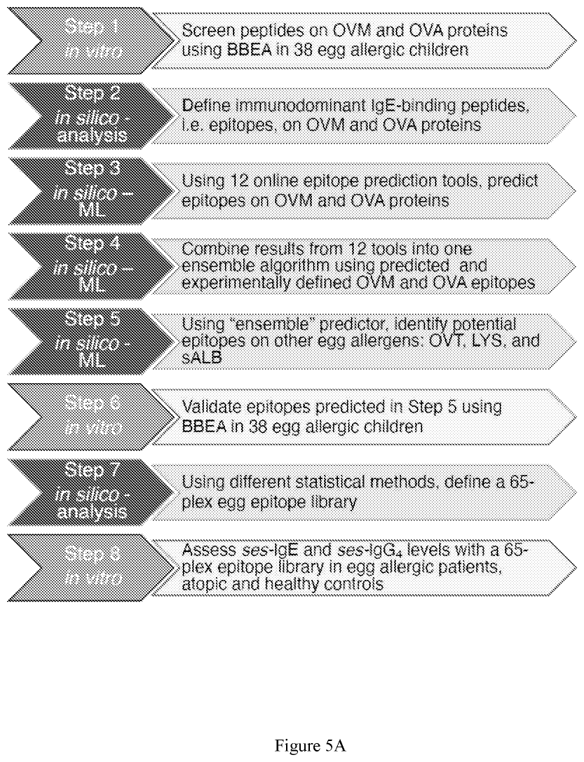

On average, egg allergic children had higher ses-IgE and lower ses-IgG4 compared to both atopic and healthy controls; the healthy subjects had almost non-detectable ses-IgE levels (see, FIG. 4A). Differences were observed primarily in OVM and some OVA epitopes.

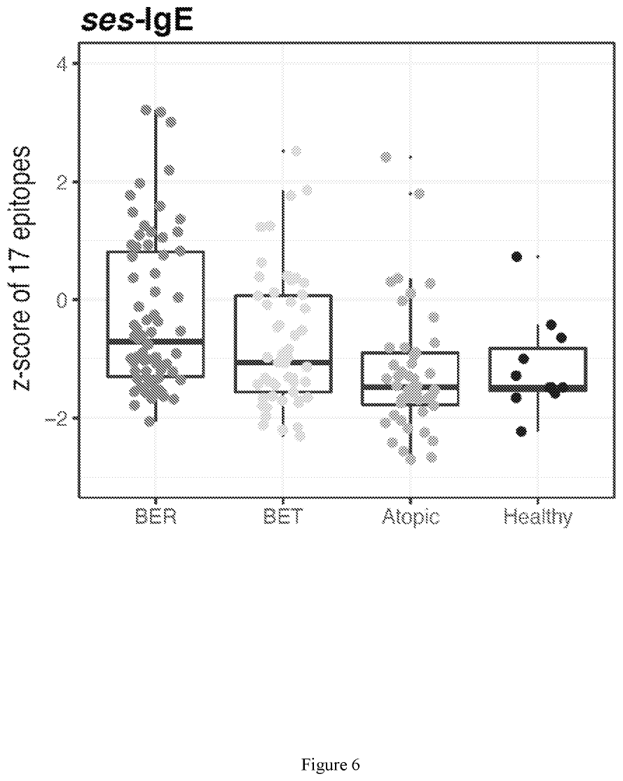

When egg allergic subjects were stratified by reactivity to baked-egg, higher OVM- and OVA-sIgE (see, FIG. 4D), as well as ses-IgE (including epitopes from minor allergens) were associated with reactive phenotype (see, FIG. 4B). This relationship was also observed for a combined z-score of either 17 significant or all 65 IgE epitopes (see, FIG. 4C, FIG. 6, and Table 3). No differences were observed between the BET patients compared to atopic or healthy controls. While OVM- and OVA-sIgG4 levels were higher in BET than BER, no differences were detected in ses-IgG4 levels (see, FIG. 4B, FIG. 4C, and FIG. 4D).

B-cells are capable of generating a specific antibody response against up to 10¹⁵ unique molecules (Rees, MAbs, 2020, 12, 1729683), making epitope mapping an arduous task. Many experimental methods can be used for functional epitope identification, including competition, antigen fragmentation or modification, and peptide library assays (Potocnakova et al., J. Immunol. Res., 2016, 2016, 6760830). Screening peptide libraries is a common approach in food allergy research since it allows simultaneous evaluation of many epitope targets. Common techniques include PEPSCAN, SPOT membranes, peptide microarrays, bead-based, or phage immunoprecipitation assays. BBEA technology is more desired herein due to the higher sensitivity, reproducibility, rapid throughput and simpler laboratory protocols of the BBEA (Suprun et al., Sci. Rep., 2019, 9, 18425; and Suprun et al., J. Allergy Clin. Immunol., 2020, 146, 1081-1088). Using this assay, all sequential peptides along the OVM and OVA backbone, the two most allergenic proteins of egg-white, were screened. Epitopes on OVM that have been identified previously (Cooke et al., J. Immunol., 1997, 159, 2026-2032; Jarvinen et al., Allergy, 2007, 62, 758-765; Holen et al., Clin. Exp. Allergy, 2001, 31, 952-964; Kovacs-Nolan et al., J. Agric. Food Chem., 2000, 48, 6261-6266; Takagi et al., Int. Arch. Allergy Immunol., 2005, 136, 23-32; Mine et al., Biochem. Biophys. Res. Commun., 2002, 292, 1070-1074; Martinez-Botas et al., Int. Arch. Allergy Immunol., 2013, 161, 11-20; Besler et al., Internet Symposium on Food Allergens (world wide web at “food-allergensde”) 1999, 1, 1-12) were confirmed, and several OVA epitopes were detected. It was expected that a small number of patients have detectable IgE to minor allergens—OVT, LYS, sALB, YGP42, and YGP40—and given that combined, those allergens would constitute >450 peptides. Accordingly, an epitope screening was conducted in silico.

To date, the most optimistic performances among a range of structure- and sequence-based in silico methods is at best modest, with an area under the curve metric usually below 0.70 (Potocnakova et al., J. Immunol. Res., 2016, 2016, 6760830; and Ponomarenko et al., BMC Struct. Biol., 2007, 7, 64). Since epitope databases used in building these methods are derived from an aggregate of bacterial, viral, fungal, environmental and auto antigens, which usually encounter the immune system in an intact form (Potocnakova et al., J. Immunol. Res., 2016, 2016, 6760830), the applicability to predicting epitopes on food proteins, which can be subject to physiological denaturing, is even less optimistic. In the present study, great variability among the 12 algorithms was observed with an average PPV of only 0.6. Recently, the principle of “wisdom of crowds” that utilizes best aspects of multiple algorithms, has been recognized as a more robust approach (Alizadeh et al., Intelligent Data Analysis, 2015, 19, 485-503), and when combined, the PPV of those tools increased to 0.7. Additionally, in allergy, the antibody of interest is IgE, which develops its high affinity to antigens over multiple rounds of receptor maturation (Gould et al., Int. Immunol., 2018, 30, 403-412). However, most in silico tools provide isotype-agnostic predictions, which are generally derived from data dominated by the IgG subclass (Gupta et al., Biol. Direct, 2013, 8, 27). Not surprising, in the ensemble method used herein, the most important predictor was BCIgEPred, which was created specifically for IgE-binding epitopes (Saravanan et al., Mol. Biol. (Mosk), 2018, 52, 333-343). Using a combination of experimental and computational approaches, a library of 65 potentially informative epitopes was proposed and showed that varying levels of IgE to these epitopes can be detected among egg allergic children.

Based on previous experience, 15-mer peptides were used, which is an average length of a B-cell epitope (Gershoni et al., BioDrugs, 2007, 21, 145-156; Sanchez-Trincado et al., J. Immunol. Res., 2017, 2017, 2680160; and Potocnakova et al., J. Immunol. Res., 2016, 2016, 6760830). While there is a possibility that shorter or longer peptides could have better IgE binding, as observed in a study using peptide microarrays 71, a goal herein was to create an epitope library that is standardized and experimentally viable. Sequential epitopes were a focus since currently there is no reliable way of testing conformational epitopes, other than using whole antigens that contain both epitope types and competing out the overall binding effect of sequential epitopes. Additionally, it has been shown that persistent egg and milk allergy is associated with both the type and number of sequential epitopes (Jarvinen et al., Allergy, 2007, 62, 758-765; Berin et al., J. Allergy Clin. Immunol., 2018, 142, 149-158 e148; Jarvinen et al., J. Allergy Clin. Immunol., 2002, 110, 293-297; Suarez-Farinas et al., J. Allergy Clin. Immunol., 2018, 143, 1038-1046; and Sackesen et al., Allergy, 2019, 74, 327-336).

The persistence of egg allergy is usually associated with reactivity to baked egg. In fact, about 70% to 80% of egg allergic children are able to tolerate heat-denatured, baked-egg products (Urisu et al., J. Allergy Clin. Immunology, 1997, 100, 171-176; Nowak-Wegrzyn et al., Curr. Opin. Allergy Clin. Immunol., 2009, 9, 234-237; Lemon-Mule et al., J. Allergy Clin. Immunology, 2008, 122, 977-983 e971; Martos et al., J. Allergy Clin. Immunology, 2011, 127, 990-997 e991-992; Des Roches et al., Allergy, 2006, 61, 900-901; Konstantinou et al., J. Allergy Clin. Immunology, 2008, 122, 414-415; and Lieberman et al., J. Allergy Clin. Immunol., 2012, 129, 1682-1684 e1682) and most of these will tolerate all forms of egg by 5 years of age (Peters et al., J. Allergy Clin. Immunol., 2014, 133, 485-491), if baked-egg is incorporated in their diet (Leonard et al., Immunol. Allergy Clin. North Am., 2016, 36, 147-159; Leonard et al., J. Allergy Clin. Immunol., 2012, 130, 473-480 e471; and Clark et al., Clin. Exp. Allergy, 2011, 41, 706-712). Thus, knowing epitope profiles that trigger pathogenic IgE could provide useful diagnostic information and inform therapeutic decisions. Additionally, increases in egg-specific IgG4 antibodies have been observed in patients desensitized by immunotherapy (Burks et al., N. Engl. J. Med., 2012, 367, 233-243; Martin-Munoz et al., Pediatr. Allergy Immunol., 2018, 30, 81-92; Romantsik et al., Cochrane Database Syst. Rev., 2018, 4, CD010638; Maeta et al., Int. Arch. Allergy Immunol., 2018, 175, 70-76; Perez-Rangel et al., Ann. Allergy Asthma Immunol., 2017, 118, 356-364 e353; Yanagida et al., Int. Arch. Allergy Immunol., 2016, 171, 265-268; and Strait et al., J. Clin. Invest., 2006, 116, 833-841) and their epitope-specific profiles could be associated with allergic phenotypes. Using the novel epitope library developed herein, ses-IgE and ses-IgG4 in baked-egg reactive and tolerant children were compared, as well as atopic and healthy subjects. Egg allergic subjects had higher levels of ses-IgE, especially to OVM epitopes, compared to the control groups, but lower OVM and OVA ses-IgG4 levels. Both atopic and healthy children had comparable ses-IgG4, likely reflecting egg consumption by children in those groups (Stapel et al., Allergy, 2008, 63, 793-796). When egg allergic children were stratified by baked-egg reactivity, the baked-egg tolerant group had similar ses-IgE to that of the controls, while baked-egg reactive subjects had a wide diversity of ses-IgE antibodies. Similarly, it was previously shown that compared to egg tolerant children, baked-egg reactive children had higher levels of sIgE to OVM, OVA, and egg-white proteins, with no differences in egg-specific IgG or egg reactive T-cells (Berin et al., J. Allergy Clin. Immunology, 2018, 142, 149-158 e148). It is important to note that while on average both sIgE and ses-IgE were higher in BER, there was a wide overlap between the two groups, suggesting that additional mechanisms contribute to this pathogenesis.

In the initial testing for the epitope library selection, low levels of ses-IgE to minor allergens we detected in a small number of subjects. When evaluated on baked-egg reactive and tolerant subjects, most differences were observed for the epitopes on OVM and OVA allergens. While IgE sensitization to proteins in yolk were shown to be helpful in prognosis of allergy persistence (Dang et al., Allergy, 2019, 74, 318-326) or low egg-white to egg-yolk IgE ratios could predict children who are able to consume hard-boiled eggs (Brossard et al., Ped. Allergy Immunol., 2019, 30, 225-233), there was a large heterogeneity among sIgE levels and skin prick test positivity. It is possible that IgE to minor allergens could be more informative when measured against whole antigens, preserving conformational epitopes; or there may be other sequential epitopes that are not represented in the epitope library.

Overall, an epitope library was created herein with experimentally derived epitopes from major OVM and OVA allergens and included a set of computationally derived epitopes from minor allergens—OVT, LYS, sALB, YGP42 and YGP40. It was demonstrated that ses-IgE is associated with BER phenotype.

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety.

What is claimed is:

1. A composition comprising up to about 65 egg peptides from 1 to 7 egg proteins,
wherein the egg proteins are selected from ovomucoid, ovalbumin, ovotransferrin, lysozyme, yolk serum albumin, YGP40, and YBG42;
wherein the ovomucoid peptides are selected from: OVM-001 (AEVDCSRFPNATDKE; SEQ ID NO: 1), OVM-003 (RFPNATDKEGKDVLV; SEQ ID NO: 2), OVM-004 (NATDKEGKDVLVCNK; SEQ ID NO: 3), OVM-005 (DKEGKDVLVCNKDLR; SEQ ID NO: 4), OVM-007 (VLVCNKDLRPICGTD; SEQ ID NO: 5), OVM-008 (CNKDLRPICGTDGVT; SEQ ID NO: 6), OVM-009 (DLRPICGTDGVTYTN; SEQ ID NO: 7), OVM-010 (PICGTDGVTYTNDCL; SEQ ID NO: 8), OVM-013 (YTNDCLLCAYSIEFG; SEQ ID NO: 9), OVM-014 (DCLLCAYSIEFGTNI; SEQ ID NO: 10), OVM-015 (LCAYSIEFGTNISKE; SEQ ID NO: 11), OVM-018 (TNISKEHDGECKETV; SEQ ID NO: 12), OVM-021 (ECKETVPMNCSSYAN; SEQ ID NO: 13), OVM-023 (PMNCSSYANTTSEDG; SEQ ID NO: 14), OVM-025 (YANTTSEDGKVMVLC; SEQ ID NO: 15), OVM-027 (EDGKVMVLCNRAFNP; SEQ ID NO: 16), OVM-028 (KVMVLCNRAFNPVCG; SEQ ID NO: 17), OVM-031 (FNPVCGTDGVTYDNE; SEQ ID NO: 18), OVM-032 (VCGTDGVTYDNECLL; SEQ ID NO: 19), OVM-033 (TDGVTYDNECLLCAH; SEQ ID NO: 20), OVM-036 (CLLCAHKVEQGASVD; SEQ ID NO: 21), OVM-038 (KVEQGASVDKRHDGG; SEQ ID NO: 22), OVM-040 (SVDKRHDGGCRKELA; SEQ ID NO: 23), OVM-041 (KRHDGGCRKELAAVS; SEQ ID NO: 24), OVM-042 (DGGCRKELAAVSVDC; SEQ ID NO: 25), OVM-044 (ELAAVSVDCSEYPKP; SEQ ID NO: 26), OVM-046 (VDCSEYPKPDCTAED; SEQ ID NO: 27), OVM-048 (PKPDCTAEDRPLCGS; SEQ ID NO: 28), OVM-050 (AEDRPLCGSDNKTYG; SEQ ID NO: 29), OVM-052 (CGSDNKTYGNKCNFC; SEQ ID NO: 30), OVM-054 (TYGNKCNFCNAVVES; SEQ ID NO: 31), OVM-055 (NKCNFCNAVVESNGT; SEQ ID NO: 32), OVM-056 (NFCNAVVESNGTLTL; SEQ ID NO: 33), and/or OVM-058 (VESNGTLTLSHFGKC; SEQ ID NO: 34);
wherein the ovalbumin peptides are selected from: OVA-001 (MGSIGAASMEFCFDV; SEQ ID NO: 35), OVA-003 (ASMEFCFDVFKELKV; SEQ ID NO: 36), OVA-006 (FKELKVHHANENIFY; SEQ ID NO: 37), OVA-021 (DKLPGFGDSIEAQCG; SEQ ID NO: 38), OVA-028 (SSLRDILNQITKPND; SEQ ID NO: 39), OVA-040 (YLQCVKELYRGGLEP; SEQ ID NO: 40), OVA-053 (IIRNVLQPSSVDSQT; SEQ ID NO: 41), OVA-057 (SQTAXVLVNAIVFKG; SEQ ID NO: 42), OVA-059 (LVNAIVFKGLWEKAF; SEQ ID NO: 43), OVA-064 (KDEDTQAMPFRVTEQ; SEQ ID NO: 44), OVA-068 (TEQESKPVQMMYQIG; SEQ ID NO: 45), OVA-071 (MMYQIGLFRVASMAS; SEQ ID NO: 46), OVA-075 (MASEKMKILELPFAS; SEQ ID NO: 47), OVA-085 (LEQLESIINFEKLTE; SEQ ID NO: 48), OVA-090 (WTSSNVMEERKIKVY; SEQ ID NO: 49), OVA-095 (LPRMKMEEKYNLTSV; SEQ ID NO: 50), OVA-108 (LKISQAVHAAHAEIN; SEQ ID NO: 51), OVA-113 (EAGREVVGSAEAGVD; SEQ ID NO: 52), and/or OVA-120 (EFRADHPFLFCIKHI; SEQ ID NO: 53);
wherein the lysozyme peptides are selected from: LYS-1 (AAAMKRHGLDNYRGY; SEQ ID NO: 54), LYS-2 (GYSLGNWVCAAKFES; SEQ ID NO: 55), LYS-3 (RNTDGSTDYGILQIN; SEQ ID NO: 56), LYS-4 (TDYGILQINSRWWCN; SEQ ID NO: 57), LYS-5 (WCNDGRTPGSRNLCN; SEQ ID NO: 58), and/or LYS-6 (WVAWRNRCKGTDVQA; SEQ ID NO: 59);
wherein the ovotransferrin peptides are selected from: OVT-1 (SSPEEKKCNNLRDLT; SEQ ID NO: 60), OVT-2 (LRDLTQQERISLTCV; SEQ ID NO: 61), OVT-3 (TIEQKLCRQCKGDPK; SEQ ID NO: 62), OVT-4 (DQLTPSPRENRIQWC; SEQ ID NO: 63), and/or OVT-5 (VMDYRECNLAEVPTH; SEQ ID NO: 64);
wherein the serum albumin peptides are selected from: sALB-1 (YQRPASDVICQEYQD SEQ ID NO: 65 or LRDSYGAMADCCSKA SEQ ID NO: 66), sALB-2 (LEKCCKTDNPAECYA; SEQ ID NO: 67), sALB-3 (KQETTPINDNVSQCC; SEQ ID NO: 68), sALB-4 (GVDTKYVPPPFNPDM; SEQ ID NO: 69), and/or sALB-5 (MVDKCCKQSDINTCFGE; SEQ ID NO: 70);
wherein the YGP40 peptides are selected from: YGP40-1 (NYSMPANCYHILVQD; SEQ ID NO: 71), YGP40-2 (VQDCSSELKFLVMMK; SEQ ID NO: 72), YGP40-3 (CAKGCSATKTTPVTV; SEQ ID NO: 73), YGP40-4 (CSATKTTPVTVGFHC; SEQ ID NO: 74), and/or YGP40-5 (FHCLPADSANSLTDK; SEQ ID NO: 75); and
wherein the YBG42 peptides are selected from: YGP42-1 (VDHQSLSREVHINTS; SEQ ID NO: 76), YGP42-2 (GVCGNNDREKHNELL; SEQ ID NO: 77), and/or YGP42-3 (SSDKKSASEDVVESV; SEQ ID NO: 78);
wherein the amino acid sequence of any one or more of the egg peptides can have one to four conservative amino acid substitutions therein.

2. The composition according to claim 1, wherein the ovomucoid peptides are selected from: OVM-014, OVM-004, OVM-009, OVM-032, and/or OVM-031.

3. The composition according to claim 1, wherein the ovomucoid peptides are selected from: OVM-014, OVM-004, OVM-009, OVM-032, and/or OVM-013.

4. The composition according to any one of claims 1 to 3, wherein the ovalbumin peptides are selected from: OVA-064, OVA-108, and/or OVA-028.

5. The composition according to any one of claims 1 to 4, wherein the lysozyme peptides are selected from: LYS-1, LYS-3, and/or LYS-6.

6. The composition according to any one of claims 1 to 5, wherein the ovotransferrin peptides are selected from: OVT-1, OVT-3, and/or OVT-5.

7. The composition according to any one of claims 1 to 6, wherein the serum albumin peptides are selected from: sALB-1, sALB-3 and/or sALB-4.

8. The composition according to any one of claims 1 to 7, wherein the YGP40 peptides are YGP40-1 and/or YGP40-5.

9. The composition according to any one of claims 1 to 8, wherein the YBG42 peptide is YGP42-1.

10. The composition according to claim 1, wherein the composition comprises the egg peptides: OVM-001, OVM-003, OVM-004, OVM-005, OVM-007, OVM-008, OVM-009, OVM-010, OVM-013, OVM-014, OVM-015, OVM-018, OVM-021, OVM-023, OVM-025, OVM-027, OVM-028, OVM-031, OVM-032, OVM-033, OVM-036, OVM-038, OVM-040, OVM-041, OVM-042, OVM-044, OVM-046, OVM-048, OVM-050, OVM-052, OVM-054, OVM-055, OVM-056, OVM-058, OVA-001, OVA-003, OVA-006, OVA-021, OVA-028, OVA-040, OVA-053, OVA-057, OVA-059, OVA-064, OVA-068, OVA-071, OVA-075, OVA-085, OVA-090, OVA-095, OVA-108, OVA-113, OVA-120, OVT-1, OVT-3, OVT-5, LYS-1, LYS-3, LYS-6, sALB-1, sALB-4, sALB-5, YGP40-1, YGP40-5, and YGP42-1.

11. The composition according to claim 1, wherein the composition comprises the egg peptides: OVT-1, OVT-2, OVT-3, OVT-4, OVT-5, LYS-1, LYS-2, LYS-3, LYS-4, LYS-5, LYS-6, sALB-1, sALB-2, sALB-3, sALB-4, sALB-5, YGP42-1, YGP42-2, YGP42-3, YGP40-1, YGP40-2, YGP40-3, YGP40-4, and YGP40-5.

12. The composition according to claim 1, wherein the composition comprises the egg peptides: OVM-014, OVM-004, OVM-009, OVM-032, OVA-064, OVA-108, OVM-013, OVA-028, OVM-031, LYS-1, and LYS-3.

13. The composition according to claim 1, wherein the composition comprises the egg peptides: OVM-014, OVM-004, OVM-009, OVM-032, OVA-064, OVA-108, OVM-013, and OVA-028.

14. The composition according to any one of claims 1 to 13, wherein each egg peptide is coupled to a solid support.

15. The composition according to claim 14, wherein each of the egg peptides is coupled to the solid support by a linker-spacer.

16. The composition according to claim 15, wherein the linker-spacer comprises a linker chosen from biotin, thiol, hydrazine, and amine, and a spacer chosen from a polypeptide, an oligonucleotide, an alkyl group, and a polyethylene glycol (PEG) group.

17. The composition according to claim 16, wherein the spacer is an alkyl group or a PEG group.

18. The composition according to claim 17, wherein the alkyl group is a C₁-C₁₈alkyl group.

19. The composition according to claim 17, wherein the PEG group is PEG1 to PEG18.

20. The composition according to claim 19, wherein the PEG group is PEG12.

21. The composition according to any one of claims 14 to 20, wherein the C-terminus of each of the egg peptides is coupled to the solid support by the linker-spacer.

22. The composition according to any one of claims 14 to 20, wherein the N-terminus of each of the egg peptides is coupled to the solid support by the linker-spacer.

23. The composition according to claim 14, wherein the C-terminus of each of the egg peptides is coupled to the solid support by a biotin-PEG12 linker-spacer.

24. The composition according to any one of claims 14 to 23, wherein the solid support is a microsphere bead, glass array, silicone array, membrane, or microtiter plate.

25. The composition according to claim 24, wherein the solid support is a microsphere bead.

26. The composition according to claim 25, wherein the microsphere bead is an avidin-coupled microsphere bead.

27. A kit comprising:
a solid support coupled to a plurality up to about 65 egg peptides from 1 to 7 egg proteins,
wherein the egg proteins are selected from ovomucoid, ovalbumin, ovotransferrin, lysozyme, yolk serum albumin, YGP40, and YBG42;
wherein the ovomucoid peptides are selected from: OVM-001 (AEVDCSRFPNATDKE; SEQ ID NO: 1), OVM-003 (RFPNATDKEGKDVLV; SEQ ID NO: 2), OVM-004 (NATDKEGKDVLVCNK; SEQ ID NO: 3), OVM-005 (DKEGKDVLVCNKDLR; SEQ ID NO: 4), OVM-007 (VLVCNKDLRPICGTD; SEQ ID NO: 5), OVM-008 (CNKDLRPICGTDGVT; SEQ ID NO: 6), OVM-009 (DLRPICGTDGVTYTN; SEQ ID NO: 7), OVM-010 (PICGTDGVTYTNDCL; SEQ ID NO: 8), OVM-013 (YTNDCLLCAYSIEFG; SEQ ID NO: 9), OVM-014 (DCLLCAYSIEFGTNI; SEQ ID NO: 10), OVM-015 (LCAYSIEFGTNISKE; SEQ ID NO: 11), OVM-018 (TNISKEHDGECKETV; SEQ ID NO: 12), OVM-021 (ECKETVPMNCSSYAN; SEQ ID NO: 13), OVM-023 (PMNCSSYANTTSEDG; SEQ ID NO: 14), OVM-025 (YANTTSEDGKVMVLC; SEQ ID NO: 15), OVM-027 (EDGKVMVLCNRAFNP; SEQ ID NO: 16), OVM-028 (KVMVLCNRAFNPVCG; SEQ ID NO: 17), OVM-031 (FNPVCGTDGVTYDNE; SEQ ID NO: 18), OVM-032 (VCGTDGVTYDNECLL; SEQ ID NO: 19), OVM-033 (TDGVTYDNECLLCAH; SEQ ID NO: 20), OVM-036 (CLLCAHKVEQGASVD; SEQ ID NO: 21), OVM-038 (KVEQGASVDKRHDGG; SEQ ID NO: 22), OVM-040 (SVDKRHDGGCRKELA; SEQ ID NO: 23), OVM-041 (KRHDGGCRKELAAVS; SEQ ID NO: 24), OVM-042 (DGGCRKELAAVSVDC; SEQ ID NO: 25), OVM-044 (ELAAVSVDCSEYPKP; SEQ ID NO: 26), OVM-046 (VDCSEYPKPDCTAED; SEQ ID NO: 27), OVM-048 (PKPDCTAEDRPLCGS; SEQ ID NO: 28), OVM-050 (AEDRPLCGSDNKTYG; SEQ ID NO: 29), OVM-052 (CGSDNKTYGNKCNFC; SEQ ID NO: 30), OVM-054 (TYGNKCNFCNAVVES; SEQ ID NO: 31), OVM-055 (NKCNFCNAVVESNGT; SEQ ID NO: 32), OVM-056 (NFCNAVVESNGTLTL; SEQ ID NO: 33), and/or OVM-058 (VESNGTLTLSHFGKC; SEQ ID NO: 34);
wherein the ovalbumin peptides are selected from: OVA-001 (MGSIGAASMEFCFDV; SEQ ID NO: 35), OVA-003 (ASMEFCFDVFKELKV; SEQ ID NO: 36), OVA-006 (FKELKVHHANENIFY; SEQ ID NO: 37), OVA-021 (DKLPGFGDSIEAQCG; SEQ ID NO: 38), OVA-028 (SSLRDILNQITKPND; SEQ ID NO: 39), OVA-040 (YLQCVKELYRGGLEP; SEQ ID NO: 40), OVA-053 (IIRNVLQPSSVDSQT; SEQ ID NO: 41), OVA-057 (SQTAXVLVNAIVFKG; SEQ ID NO: 42), OVA-059 (LVNAIVFKGLWEKAF; SEQ ID NO: 43), OVA-064 (KDEDTQAMPFRVTEQ; SEQ ID NO: 44), OVA-068 (TEQESKPVQMMYQIG; SEQ ID NO: 45), OVA-071 (MMYQIGLFRVASMAS; SEQ ID NO: 46), OVA-075 (MASEKMKILELPFAS; SEQ ID NO: 47), OVA-085 (LEQLESIINFEKLTE; SEQ ID NO: 48), OVA-090 (WTSSNVMEERKIKVY; SEQ ID NO: 49), OVA-095 (LPRMKMEEKYNLTSV; SEQ ID NO: 50), OVA-108 (LKISQAVHAAHAEIN; SEQ ID NO: 51), OVA-113 (EAGREVVGSAEAGVD; SEQ ID NO: 52), and/or OVA-120 (EFRADHPFLFCIKHI; SEQ ID NO: 53);
wherein the lysozyme peptides are selected from: LYS-1 (AAAMKRHGLDNYRGY; SEQ ID NO: 54), LYS-2 (GYSLGNWVCAAKFES; SEQ ID NO: 55), LYS-3 (RNTDGSTDYGILQIN; SEQ ID NO: 56), LYS-4 (TDYGILQINSRWWCN; SEQ ID NO: 57), LYS-5 (WCNDGRTPGSRNLCN; SEQ ID NO: 58), and/or LYS-6 (WVAWRNRCKGTDVQA; SEQ ID NO: 59);
wherein the ovotransferrin peptides are selected from: OVT-1 (SSPEEKKCNNLRDLT; SEQ ID NO: 60), OVT-2 (LRDLTQQERISLTCV; SEQ ID NO: 61), OVT-3 (TIEQKLCRQCKGDPK; SEQ ID NO: 62), OVT-4 (DQLTPSPRENRIQWC; SEQ ID NO: 63), and/or OVT-5 (VMDYRECNLAEVPTH; SEQ ID NO: 64);
wherein the serum albumin peptides are selected from: sALB-1 (YQRPASDVICQEYQD SEQ ID NO: 65 or LRDSYGAMADCCSKA SEQ ID NO: 66), sALB-2 (LEKCCKTDNPAECYA; SEQ ID NO: 67), sALB-3 (KQETTPINDNVSQCC; SEQ ID NO: 68), sALB-4 (GVDTKYVPPPFNPDM; SEQ ID NO: 69), and/or sALB-5 (MVDKCCKQSDINTCFGE; SEQ ID NO: 70);
wherein the YGP40 peptides are selected from: YGP40-1 (NYSMPANCYHILVQD; SEQ ID NO: 71), YGP40-2 (VQDCSSELKFLVMMK; SEQ ID NO: 72), YGP40-3 (CAKGCSATKTTPVTV; SEQ ID NO: 73), YGP40-4 (CSATKTTPVTVGFHC; SEQ ID NO: 74), and/or YGP40-5 (FHCLPADSANSLTDK; SEQ ID NO: 75); and
wherein the YBG42 peptides are selected from: YGP42-1 (VDHQSLSREVHINTS; SEQ ID NO: 76), YGP42-2 (GVCGNNDREKHNELL; SEQ ID NO: 77), and/or YGP42-3 (SSDKKSASEDVVESV; SEQ ID NO: 78);
wherein the amino acid sequence of any one or more of the egg peptides can have one to four conservative amino acid substitutions therein; and
an allergy associated immunoglobulin (AAI)-specific labeling reagent.

28. The kit according to claim 27, wherein the ovomucoid peptides are selected from: OVM-014, OVM-004, OVM-009, OVM-032, and/or OVM-031.

29. The kit according to claim 27, wherein the ovomucoid peptides are selected from: OVM-014, OVM-004, OVM-009, OVM-032, and/or OVM-013.

30. The kit according to any one of claims 27 to 29, wherein the ovalbumin peptides are selected from: OVA-064, OVA-108, and/or OVA-028.

31. The kit according to any one of claims 27 to 30, wherein the lysozyme peptides are selected from: LYS-1, LYS-3, and/or LYS-6.

32. The kit according to any one of claims 27 to 31, wherein the ovotransferrin peptides are selected from: OVT-1, OVT-3, and/or OVT-5.

33. The kit according to any one of claims 27 to 32, wherein the serum albumin peptides are selected from: sALB-1, sALB-3 and/or sALB-4.

34. The kit according to any one of claims 27 to 33, wherein the YGP40 peptides are YGP40-1 and/or YGP40-5.

35. The kit according to any one of claims 27 to 34, wherein the YBG42 peptide is YGP42-1.

36. The kit according to claim 27, wherein the composition comprises the egg peptides: OVM-001, OVM-003, OVM-004, OVM-005, OVM-007, OVM-008, OVM-009, OVM-010, OVM-013, OVM-014, OVM-015, OVM-018, OVM-021, OVM-023, OVM-025, OVM-027, OVM-028, OVM-031, OVM-032, OVM-033, OVM-036, OVM-038, OVM-040, OVM-041, OVM-042, OVM-044, OVM-046, OVM-048, OVM-050, OVM-052, OVM-054, OVM-055, OVM-056, OVM-058, OVA-001, OVA-003, OVA-006, OVA-021, OVA-028, OVA-040, OVA-053, OVA-057, OVA-059, OVA-064, OVA-068, OVA-071, OVA-075, OVA-085, OVA-090, OVA-095, OVA-108, OVA-113, OVA-120, OVT-1, OVT-3, OVT-5, LYS-1, LYS-3, LYS-6, sALB-1, sALB-4, sALB-5, YGP40-1, YGP40-5, and YGP42-1.

37. The kit according to claim 27, wherein the composition comprises the egg peptides: OVT-1, OVT-2, OVT-3, OVT-4, OVT-5, LYS-1, LYS-2, LYS-3, LYS-4, LYS-5, LYS-6, sALB-1, sALB-2, sALB-3, sALB-4, sALB-5, YGP42-1, YGP42-2, YGP42-3, YGP40-1, YGP40-2, YGP40-3, YGP40-4, and YGP40-5.

38. The kit according to claim 27, wherein the composition comprises the egg peptides: OVM-014, OVM-004, OVM-009, OVM-032, OVA-064, OVA-108, OVM-013, OVA-028, OVM-031, LYS-1, and LYS-3.

39. The kit according to claim 27, wherein the composition comprises the egg peptides: OVM-014, OVM-004, OVM-009, OVM-032, OVA-064, OVA-108, OVM-013, and OVA-028.

40. The kit according to any one of claims 27 to 39, further comprising instructions for use.

41. The kit according to any one of claims 27 to 40, further comprising one or more of a binding buffer, a wash buffer, a detection buffer, a non-allergic control sample, a negative buffer control sample, and an allergic positive control sample.

42. The kit according to any one of claims 27 to 41, wherein each of the egg peptides is coupled to the solid support by a linker-spacer.

43. The kit according to claim 42, wherein the linker-spacer comprises a linker chosen from biotin, thiol, hydrazine, and amine, and a spacer chosen from a polypeptide, an oligonucleotide, an alkyl group, and a polyethylene glycol (PEG) group.

44. The kit according to claim 43, wherein the spacer is an alkyl group or a PEG group.

45. The kit according to claim 44, wherein the alkyl group is a C₁-C₁₈alkyl group.

46. The kit according to claim 44, wherein the PEG group is PEG1 to PEG18.

47. The kit according to claim 46, wherein the PEG group is PEG12.

48. The kit according to any one of claims 27 to 47, wherein the C-terminus of each of the egg peptides is coupled to the solid support by the linker-spacer.

49. The kit according to any one of claims 27 to 47, wherein the N-terminus of each of the egg peptides is coupled to the solid support by the linker-spacer.

50. The kit according to claim 42, wherein the C-terminus of each of the egg peptides is coupled to the solid support by a biotin-PEG12 linker-spacer.

51. The kit according to any one of claims 38 to 50, wherein the solid support is a microsphere bead, glass array, silicone array, membrane, or microtiter plate.

52. The kit according to claim 51, wherein the solid support is a microsphere bead.

53. The kit according to claim 52, wherein the microsphere bead is an avidin-coupled microsphere bead.

54. The kit according to any one of claims 38 to 53, wherein the AAI-specific labeling reagent is a detectably labeled anti-human antibody.

55. The kit according to claim 54, wherein the detectably labeled anti-human antibody is detectably labeled anti-human IgE antibody.

56. The kit according to claim 54 or claim 55, wherein the detectable label of the AAI-specific labeling reagent is chosen from phycoerythrin (PE), a cyanine dye, a fluorescent dye, an infrared dye, a chromogenic dye, an enzyme label, and a radioactive label.

57. The kit according to claim 56, wherein the detectable label is PE.

58. The kit according to claim 54, wherein the AAI-specific labeling reagent is a PE-labeled anti-human IgE antibody.

59. The kit according to any one of claims 38 to 58, further comprising a reporter moiety that specifically binds to the AAI-specific labeling reagent.

60. A method for diagnosing an egg allergy in a subject comprising:
contacting a plurality of egg peptides coupled to a solid support with a biological sample obtained from the subject under conditions sufficient to permit binding of one or more allergy associated immunoglobulins (AAIs) in the biological sample to egg peptides to form AAI-peptide-solid support complexes, wherein the plurality of egg peptides comprises up to about 65 egg peptides from 1 to 7 egg proteins,
wherein the egg proteins are selected from ovomucoid, ovalbumin, ovotransferrin, lysozyme, yolk serum albumin, YGP40, and YBG42;
wherein the ovomucoid peptides are selected from: OVM-001 (AEVDCSRFPNATDKE; SEQ ID NO: 1), OVM-003 (RFPNATDKEGKDVLV; SEQ ID NO: 2), OVM-004 (NATDKEGKDVLVCNK; SEQ ID NO: 3), OVM-005 (DKEGKDVLVCNKDLR; SEQ ID NO: 4), OVM-007 (VLVCNKDLRPICGTD; SEQ ID NO: 5), OVM-008 (CNKDLRPICGTDGVT; SEQ ID NO: 6), OVM-009 (DLRPICGTDGVTYTN; SEQ ID NO: 7), OVM-010 (PICGTDGVTYTNDCL; SEQ ID NO: 8), OVM-013 (YTNDCLLCAYSIEFG; SEQ ID NO: 9), OVM-014 (DCLLCAYSIEFGTNI; SEQ ID NO: 10), OVM-015 (LCAYSIEFGTNISKE; SEQ ID NO: 11), OVM-018 (TNISKEHDGECKETV; SEQ ID NO: 12), OVM-021 (ECKETVPMNCSSYAN; SEQ ID NO: 13), OVM-023 (PMNCSSYANTTSEDG; SEQ ID NO: 14), OVM-025 (YANTTSEDGKVMVLC; SEQ ID NO: 15), OVM-027 (EDGKVMVLCNRAFNP; SEQ ID NO: 16), OVM-028 (KVMVLCNRAFNPVCG; SEQ ID NO: 17), OVM-031 (FNPVCGTDGVTYDNE; SEQ ID NO: 18), OVM-032 (VCGTDGVTYDNECLL; SEQ ID NO: 19), OVM-033 (TDGVTYDNECLLCAH; SEQ ID NO: 20), OVM-036 (CLLCAHKVEQGASVD; SEQ ID NO: 21), OVM-038 (KVEQGASVDKRHDGG; SEQ ID NO: 22), OVM-040 (SVDKRHDGGCRKELA; SEQ ID NO: 23), OVM-041 (KRHDGGCRKELAAVS; SEQ ID NO: 24), OVM-042 (DGGCRKELAAVSVDC; SEQ ID NO: 25), OVM-044 (ELAAVSVDCSEYPKP; SEQ ID NO: 26), OVM-046 (VDCSEYPKPDCTAED; SEQ ID NO: 27), OVM-048 (PKPDCTAEDRPLCGS; SEQ ID NO: 28), OVM-050 (AEDRPLCGSDNKTYG; SEQ ID NO: 29), OVM-052 (CGSDNKTYGNKCNFC; SEQ ID NO: 30), OVM-054 (TYGNKCNFCNAVVES; SEQ ID NO: 31), OVM-055 (NKCNFCNAVVESNGT; SEQ ID NO: 32), OVM-056 (NFCNAVVESNGTLTL; SEQ ID NO: 33), and/or OVM-058 (VESNGTLTLSHFGKC; SEQ ID NO: 34);
wherein the ovalbumin peptides are selected from: OVA-001 (MGSIGAASMEFCFDV; SEQ ID NO: 35), OVA-003 (ASMEFCFDVFKELKV; SEQ ID NO: 36), OVA-006 (FKELKVHHANENIFY; SEQ ID NO: 37), OVA-021 (DKLPGFGDSIEAQCG; SEQ ID NO: 38), OVA-028 (SSLRDILNQITKPND; SEQ ID NO: 39), OVA-040 (YLQCVKELYRGGLEP; SEQ ID NO: 40), OVA-053 (IIRNVLQPSSVDSQT; SEQ ID NO: 41), OVA-057 (SQTAXVLVNAIVFKG; SEQ ID NO: 42), OVA-059 (LVNAIVFKGLWEKAF; SEQ ID NO: 43), OVA-064 (KDEDTQAMPFRVTEQ; SEQ ID NO: 44), OVA-068 (TEQESKPVQMMYQIG; SEQ ID NO: 45), OVA-071 (MMYQIGLFRVASMAS; SEQ ID NO: 46), OVA-075 (MASEKMKILELPFAS; SEQ ID NO: 47), OVA-085 (LEQLESIINFEKLTE; SEQ ID NO: 48), OVA-090 (WTSSNVMEERKIKVY; SEQ ID NO: 49), OVA-095 (LPRMKMEEKYNLTSV; SEQ ID NO: 50), OVA-108 (LKISQAVHAAHAEIN; SEQ ID NO: 51), OVA-113 (EAGREVVGSAEAGVD; SEQ ID NO: 52), and/or OVA-120 (EFRADHPFLFCIKHI; SEQ ID NO: 53);
wherein the lysozyme peptides are selected from: LYS-1 (AAAMKRHGLDNYRGY; SEQ ID NO: 54), LYS-2 (GYSLGNWVCAAKFES; SEQ ID NO: 55), LYS-3 (RNTDGSTDYGILQIN; SEQ ID NO: 56), LYS-4 (TDYGILQINSRWWCN; SEQ ID NO: 57), LYS-5 (WCNDGRTPGSRNLCN; SEQ ID NO: 58), and/or LYS-6 (WVAWRNRCKGTDVQA; SEQ ID NO: 59);
wherein the ovotransferrin peptides are selected from: OVT-1 (SSPEEKKCNNLRDLT; SEQ ID NO: 60), OVT-2 (LRDLTQQERISLTCV; SEQ ID NO: 61), OVT-3 (TIEQKLCRQCKGDPK; SEQ ID NO: 62), OVT-4 (DQLTPSPRENRIQWC; SEQ ID NO: 63), and/or OVT-5 (VMDYRECNLAEVPTH; SEQ ID NO: 64);
wherein the serum albumin peptides are selected from: sALB-1 (YQRPASDVICQEYQD SEQ ID NO: 65 or LRDSYGAMADCCSKA SEQ ID NO: 66), sALB-2 (LEKCCKTDNPAECYA; SEQ ID NO: 67), sALB-3 (KQETTPINDNVSQCC; SEQ ID NO: 68), sALB-4 (GVDTKYVPPPFNPDM; SEQ ID NO: 69), and/or sALB-5 (MVDKCCKQSDINTCFGE; SEQ ID NO: 70);
wherein the YGP40 peptides are selected from: YGP40-1 (NYSMPANCYHILVQD; SEQ ID NO: 71), YGP40-2 (VQDCSSELKFLVMMK; SEQ ID NO: 72), YGP40-3 (CAKGCSATKTTPVTV; SEQ ID NO: 73), YGP40-4 (CSATKTTPVTVGFHC; SEQ ID NO: 74), and/or YGP40-5 (FHCLPADSANSLTDK; SEQ ID NO: 75); and
wherein the YBG42 peptides are selected from: YGP42-1 (VDHQSLSREVHINTS; SEQ ID NO: 76), YGP42-2 (GVCGNNDREKHNELL; SEQ ID NO: 77), and/or YGP42-3 (SSDKKSASEDVVESV; SEQ ID NO: 78);
wherein the amino acid sequence of any one or more of the egg peptides can have one to four conservative amino acid substitutions therein;
contacting the AAI-peptide-solid support complexes with an AAI-specific labeling reagent to form labeling reagent-AAI-peptide-solid support complexes; and
measuring the binding of the AAI-specific labeling reagent to each AAI-peptide-solid support complex;
wherein when the combined binding for each egg peptide in the AAI-peptide solid support complex to the AAI-specific labeling reagent is greater than a threshold value, the subject is allergic to eggs, and when the combined binding for each egg peptide in the AAI-peptide solid support complex to the AAI-specific labeling reagent is equal to or less than the threshold value, the subject is not allergic to eggs.

61. The method according to claim 60, wherein the ovomucoid peptides are selected from: OVM-014, OVM-004, OVM-009, OVM-032, and/or OVM-031.

62. The method according to claim 60, wherein the ovomucoid peptides are selected from: OVM-014, OVM-004, OVM-009, OVM-032, and/or OVM-013.

63. The method according to any one of claims 60 to 62, wherein the ovalbumin peptides are selected from: OVA-064, OVA-108, and/or OVA-028.

64. The method according to any one of claims 60 to 63, wherein the lysozyme peptides are selected from: LYS-1, LYS-3, and/or LYS-6.

65. The method according to any one of claims 60 to 64, wherein the ovotransferrin peptides are selected from: OVT-1, OVT-3, and/or OVT-5.

66. The method according to any one of claims 60 to 65, wherein the serum albumin peptides are selected from: sALB-1, sALB-3 and/or sALB-4.

67. The method according to any one of claims 60 to 66, wherein the YGP40 peptides are YGP40-1 and/or YGP40-5.

68. The method according to any one of claims 60 to 67, wherein the YBG42 peptide is YGP42-1.

69. The method according to claim 60, wherein the composition comprises the egg peptides: OVM-001, OVM-003, OVM-004, OVM-005, OVM-007, OVM-008, OVM-009, OVM-010, OVM-013, OVM-014, OVM-015, OVM-018, OVM-021, OVM-023, OVM-025, OVM-027, OVM-028, OVM-031, OVM-032, OVM-033, OVM-036, OVM-038, OVM-040, OVM-041, OVM-042, OVM-044, OVM-046, OVM-048, OVM-050, OVM-052, OVM-054, OVM-055, OVM-056, OVM-058, OVA-001, OVA-003, OVA-006, OVA-021, OVA-028, OVA-040, OVA-053, OVA-057, OVA-059, OVA-064, OVA-068, OVA-071, OVA-075, OVA-085, OVA-090, OVA-095, OVA-108, OVA-113, OVA-120, OVT-1, OVT-3, OVT-5, LYS-1, LYS-3, LYS-6, sALB-1, sALB-4, sALB-5, YGP40-1, YGP40-5, and YGP42-1.

70. The method according to claim 60, wherein the composition comprises the egg peptides: OVT-1, OVT-2, OVT-3, OVT-4, OVT-5, LYS-1, LYS-2, LYS-3, LYS-4, LYS-5, LYS-6, sALB-1, sALB-2, sALB-3, sALB-4, sALB-5, YGP42-1, YGP42-2, YGP42-3, YGP40-1, YGP40-2, YGP40-3, YGP40-4, and YGP40-5.

71. The method according to claim 60, wherein the composition comprises the egg peptides: OVM-014, OVM-004, OVM-009, OVM-032, OVA-064, OVA-108, OVM-013, OVA-028, OVM-031, LYS-1, and LYS-3.

72. The method according to claim 60, wherein the composition comprises the egg peptides: OVM-014, OVM-004, OVM-009, OVM-032, OVA-064, OVA-108, OVM-013, and OVA-028.

73. The method according to claim 72, wherein the biological sample is chosen from serum, plasma, saliva, or a buccal swab.

74. The method according to claim 72, wherein the biological sample is serum or plasma.

75. The method according to any one of claims 72 to 74, wherein each of the egg peptides is coupled to the solid support by a linker-spacer.

76. The method according to claim 75, wherein the linker-spacer comprises a linker chosen from biotin, thiol, hydrazine, and amine, and a spacer chosen from a polypeptide, an oligonucleotide, an alkyl group, and a polyethylene glycol (PEG) group.

77. The method according to claim 76, wherein the spacer is an alkyl group or a PEG group.

78. The method according to claim 77, wherein the alkyl group is a C₁-C₁₈alkyl group.

79. The method according to claim 77, wherein the PEG group is PEG1 to PEG18.

80. The method according to claim 79, wherein the PEG group is PEG12.

81. The method according to any one of claims 75 to 80, wherein the C-terminus of each of the egg peptides is coupled to the solid support by the linker-spacer.

82. The method according to any one of claims 75 to 80, wherein the N-terminus of each of the egg peptides is coupled to the solid support by the linker-spacer.

83. The method according to claim 75, wherein the C-terminus of each of the egg peptides is coupled to the solid support by a biotin-PEG12 linker-spacer.

84. The method according to any one of claims 72 to 83, wherein the solid support is a microsphere bead, glass array, silicone array, membrane, or microtiter plate.

85. The method according to claim 84, wherein the solid support is a microsphere bead.

86. The method according to claim 85, wherein the microsphere bead is an avidin-coupled microsphere bead.

87. The method according to any one of claims 72 to 86, wherein the AAI is IgM, IgA, and/or IgD.

88. The method according to any one of claims 72 to 86, wherein the AAI is IgG and/or IgE.

89. The method according to any one of claims 72 to 86, wherein the AAI is IgE.

90. The method according to any one of claims 72 to 89, wherein the AAI-specific labeling reagent is a detectably labeled anti-human antibody.

91. The method according to claim 90, wherein the detectably labeled anti-human antibody is detectably labeled anti-human IgE antibody.

92. The method according to claim 90 or claim 91, wherein the detectable label of the AAI-specific labeling reagent is chosen from phycoerythrin (PE), a cyanine dye, a fluorescent dye, an infrared dye, a chromogenic dye, an enzyme label, and a radioactive label.

93. The method according to claim 92, wherein the detectable label is PE.

94. The method according to claim 90, wherein the AAI-specific labeling reagent is a PE-labeled anti-human IgE antibody.

95. The method according to any one of claims 72 to 94, wherein the measuring of the binding of the AAI-specific labeling reagent to each AAI-peptide-solid support complex is carried out by a point of care device.

96. The method according to claim 95, wherein the point of care device is a multiplex peptide-bead flow cytometric analysis device or a lateral flow assay device.

97. The method according to any one of claims 72 to 96, wherein measuring the binding of the AAI-specific labeling reagent to each AAI-peptide-solid support complex comprises measuring the mean fluorescent intensity (MFI) of each AAI-specific labeling reagent; and
wherein the AAI-specific labeling reagent is a detectably labeled anti-human antibody.

98. The method according to claim 97, wherein the MFI of each AAI-specific labeling reagent is background subtracted.

99. The method according to any one of claims 72 to 98, further comprising performing or having performed a skin prick test (SPT) and/or a total egg specific IgE (sIgE) test.

100. The method according to claim 99, wherein when the SPT is ≤3 mm and/or the sIgE is ≤0.10 kU/L then the subject is not allergic to eggs, and when the SPT is ≥18 mm and/or the sIgE is ≥18 kU/L then the subject is allergic to eggs.

101. A method for detecting development of clinical tolerance to eggs in a subject that is allergic to eggs comprising:
contacting a plurality of egg peptides coupled to a solid support with a biological sample obtained from the subject under conditions sufficient to permit binding of one or more allergy associated immunoglobulins (AAIs) in the biological sample to egg peptides to form AAI-peptide-solid support complexes, wherein the plurality of egg peptides comprises up to about 65 egg peptides from 1 to 7 egg proteins,
wherein the egg proteins are selected from ovomucoid, ovalbumin, ovotransferrin, lysozyme, yolk serum albumin, YGP40, and YBG42;
wherein the ovomucoid peptides are selected from: OVM-001 (AEVDCSRFPNATDKE; SEQ ID NO: 1), OVM-003 (RFPNATDKEGKDVLV; SEQ ID NO: 2), OVM-004 (NATDKEGKDVLVCNK; SEQ ID NO: 3), OVM-005 (DKEGKDVLVCNKDLR; SEQ ID NO: 4), OVM-007 (VLVCNKDLRPICGTD; SEQ ID NO: 5), OVM-008 (CNKDLRPICGTDGVT; SEQ ID NO: 6), OVM-009 (DLRPICGTDGVTYTN; SEQ ID NO: 7), OVM-010 (PICGTDGVTYTNDCL; SEQ ID NO: 8), OVM-013 (YTNDCLLCAYSIEFG; SEQ ID NO: 9), OVM-014 (DCLLCAYSIEFGTNI; SEQ ID NO: 10), OVM-015 (LCAYSIEFGTNISKE; SEQ ID NO: 11), OVM-018 (TNISKEHDGECKETV; SEQ ID NO: 12), OVM-021 (ECKETVPMNCSSYAN; SEQ ID NO: 13), OVM-023 (PMNCSSYANTTSEDG; SEQ ID NO: 14), OVM-025 (YANTTSEDGKVMVLC; SEQ ID NO: 15), OVM-027 (EDGKVMVLCNRAFNP; SEQ ID NO: 16), OVM-028 (KVMVLCNRAFNPVCG; SEQ ID NO: 17), OVM-031 (FNPVCGTDGVTYDNE; SEQ ID NO: 18), OVM-032 (VCGTDGVTYDNECLL; SEQ ID NO: 19), OVM-033 (TDGVTYDNECLLCAH; SEQ ID NO: 20), OVM-036 (CLLCAHKVEQGASVD; SEQ ID NO: 21), OVM-038 (KVEQGASVDKRHDGG; SEQ ID NO: 22), OVM-040 (SVDKRHDGGCRKELA; SEQ ID NO: 23), OVM-041 (KRHDGGCRKELAAVS; SEQ ID NO: 24), OVM-042 (DGGCRKELAAVSVDC; SEQ ID NO: 25), OVM-044 (ELAAVSVDCSEYPKP; SEQ ID NO: 26), OVM-046 (VDCSEYPKPDCTAED; SEQ ID NO: 27), OVM-048 (PKPDCTAEDRPLCGS; SEQ ID NO: 28), OVM-050 (AEDRPLCGSDNKTYG; SEQ ID NO: 29), OVM-052 (CGSDNKTYGNKCNFC; SEQ ID NO: 30), OVM-054 (TYGNKCNFCNAVVES; SEQ ID NO: 31), OVM-055 (NKCNFCNAVVESNGT; SEQ ID NO: 32), OVM-056 (NFCNAVVESNGTLTL; SEQ ID NO: 33), and/or OVM-058 (VESNGTLTLSHFGKC; SEQ ID NO: 34);
wherein the ovalbumin peptides are selected from: OVA-001 (MGSIGAASMEFCFDV; SEQ ID NO: 35), OVA-003 (ASMEFCFDVFKELKV; SEQ ID NO: 36), OVA-006 (FKELKVHHANENIFY; SEQ ID NO: 37), OVA-021 (DKLPGFGDSIEAQCG; SEQ ID NO: 38), OVA-028 (SSLRDILNQITKPND; SEQ ID NO: 39), OVA-040 (YLQCVKELYRGGLEP; SEQ ID NO: 40), OVA-053 (IIRNVLQPSSVDSQT; SEQ ID NO: 41), OVA-057 (SQTAXVLVNAIVFKG; SEQ ID NO: 42), OVA-059 (LVNAIVFKGLWEKAF; SEQ ID NO: 43), OVA-064 (KDEDTQAMPFRVTEQ; SEQ ID NO: 44), OVA-068 (TEQESKPVQMMYQIG; SEQ ID NO: 45), OVA-071 (MMYQIGLFRVASMAS; SEQ ID NO: 46), OVA-075 (MASEKMKILELPFAS; SEQ ID NO: 47), OVA-085 (LEQLESIINFEKLTE; SEQ ID NO: 48), OVA-090 (WTSSNVMEERKIKVY; SEQ ID NO: 49), OVA-095 (LPRMKMEEKYNLTSV; SEQ ID NO: 50), OVA-108 (LKISQAVHAAHAEIN; SEQ ID NO: 51), OVA-113 (EAGREVVGSAEAGVD; SEQ ID NO: 52), and/or OVA-120 (EFRADHPFLFCIKHI; SEQ ID NO: 53);
wherein the lysozyme peptides are selected from: LYS-1 (AAAMKRHGLDNYRGY; SEQ ID NO: 54), LYS-2 (GYSLGNWVCAAKFES; SEQ ID NO: 55), LYS-3 (RNTDGSTDYGILQIN; SEQ ID NO: 56), LYS-4 (TDYGILQINSRWWCN; SEQ ID NO: 57), LYS-5 (WCNDGRTPGSRNLCN; SEQ ID NO: 58), and/or LYS-6 (WVAWRNRCKGTDVQA; SEQ ID NO: 59);
wherein the ovotransferrin peptides are selected from: OVT-1 (SSPEEKKCNNLRDLT; SEQ ID NO: 60), OVT-2 (LRDLTQQERISLTCV; SEQ ID NO: 61), OVT-3 (TIEQKLCRQCKGDPK; SEQ ID NO: 62), OVT-4 (DQLTPSPRENRIQWC; SEQ ID NO: 63), and/or OVT-5 (VMDYRECNLAEVPTH; SEQ ID NO: 64);
wherein the serum albumin peptides are selected from: sALB-1 (YQRPASDVICQEYQD SEQ ID NO: 65 or LRDSYGAMADCCSKA SEQ ID NO: 66), sALB-2 (LEKCCKTDNPAECYA; SEQ ID NO: 67), sALB-3 (KQETTPINDNVSQCC; SEQ ID NO: 68), sALB-4 (GVDTKYVPPPFNPDM; SEQ ID NO: 69), and/or sALB-5 (MVDKCCKQSDINTCFGE; SEQ ID NO: 70);
wherein the YGP40 peptides are selected from: YGP40-1 (NYSMPANCYHILVQD; SEQ ID NO: 71), YGP40-2 (VQDCSSELKFLVMMK; SEQ ID NO: 72), YGP40-3 (CAKGCSATKTTPVTV; SEQ ID NO: 73), YGP40-4 (CSATKTTPVTVGFHC; SEQ ID NO: 74), and/or YGP40-5 (FHCLPADSANSLTDK; SEQ ID NO: 75); and
wherein the YBG42 peptides are selected from: YGP42-1 (VDHQSLSREVHINTS; SEQ ID NO: 76), YGP42-2 (GVCGNNDREKHNELL; SEQ ID NO: 77), and/or YGP42-3 (SSDKKSASEDVVESV; SEQ ID NO: 78);
wherein the amino acid sequence of any one or more of the egg peptides can have one to four conservative amino acid substitutions therein;
contacting the AAI-peptide-solid support complexes with an AAI-specific labeling reagent to form labeling reagent-AAI-peptide-solid support complexes;
measuring the binding of the AAI-specific labeling reagent to each AAI-peptide-solid support complex; and
comparing the binding of the AAI-specific labeling reagent to each AAI-peptide-solid support complex to the binding of the AAI-specific labeling reagent to each AAI-peptide-solid support complex from a biological sample previously obtained from the subject;
wherein when the combined binding for each egg peptide in the AAI-peptide solid support complex to the AAI-specific labeling reagent is greater than or equal to the combined binding for each egg peptide in the AAI-peptide solid support complex to the AAI-specific labeling reagent for the previously obtained biological sample, the subject has not established clinical tolerance to eggs; and when the combined binding for each egg peptide in the AAI-peptide solid support complex to the AAI-specific labeling reagent is less than the combined binding for each egg peptide in the AAI-peptide solid support complex to the AAI-specific labeling reagent for the previously obtained biological sample, the subject has established clinical tolerance to eggs.

102. The method according to claim 101, wherein the ovomucoid peptides are selected from: OVM-014, OVM-004, OVM-009, OVM-032, and/or OVM-031.

103. The method according to claim 101, wherein the ovomucoid peptides are selected from: OVM-014, OVM-004, OVM-009, OVM-032, and/or OVM-013.

104. The method according to any one of claims 101 to 103, wherein the ovalbumin peptides are selected from: OVA-064, OVA-108, and/or OVA-028.

105. The method according to any one of claims 101 to 104, wherein the lysozyme peptides are selected from: LYS-1, LYS-3, and/or LYS-6.

106. The method according to any one of claims 101 to 105, wherein the ovotransferrin peptides are selected from: OVT-1, OVT-3, and/or OVT-5.

107. The method according to any one of claims 101 to 106, wherein the serum albumin peptides are selected from: sALB-1, sALB-3 and/or sALB-4.

108. The method according to any one of claims 101 to 107, wherein the YGP40 peptides are YGP40-1 and/or YGP40-5.

109. The method according to any one of claims 101 to 108, wherein the YBG42 peptide is YGP42-1.

110. The method according to claim 101, wherein the composition comprises the egg peptides: OVM-001, OVM-003, OVM-004, OVM-005, OVM-007, OVM-008, OVM-009, OVM-010, OVM-013, OVM-014, OVM-015, OVM-018, OVM-021, OVM-023, OVM-025, OVM-027, OVM-028, OVM-031, OVM-032, OVM-033, OVM-036, OVM-038, OVM-040, OVM-041, OVM-042, OVM-044, OVM-046, OVM-048, OVM-050, OVM-052, OVM-054, OVM-055, OVM-056, OVM-058, OVA-001, OVA-003, OVA-006, OVA-021, OVA-028, OVA-040, OVA-053, OVA-057, OVA-059, OVA-064, OVA-068, OVA-071, OVA-075, OVA-085, OVA-090, OVA-095, OVA-108, OVA-113, OVA-120, OVT-1, OVT-3, OVT-5, LYS-1, LYS-3, LYS-6, sALB-1, sALB-4, sALB-5, YGP40-1, YGP40-5, and YGP42-1.

111. The method according to claim 101, wherein the composition comprises the egg peptides: OVT-1, OVT-2, OVT-3, OVT-4, OVT-5, LYS-1, LYS-2, LYS-3, LYS-4, LYS-5, LYS-6, sALB-1, sALB-2, sALB-3, sALB-4, sALB-5, YGP42-1, YGP42-2, YGP42-3, YGP40-1, YGP40-2, YGP40-3, YGP40-4, and YGP40-5.

112. The method according to claim 101, wherein the composition comprises the egg peptides: OVM-014, OVM-004, OVM-009, OVM-032, OVA-064, OVA-108, OVM-013, OVA-028, OVM-031, LYS-1, and LYS-3.

113. The method according to claim 101, wherein the composition comprises the egg peptides: OVM-014, OVM-004, OVM-009, OVM-032, OVA-064, OVA-108, OVM-013, and OVA-028.

114. The method according to any one of claims 101 to 113, wherein the biological sample is chosen from serum, plasma, saliva, or a buccal swab.

115. The method according to claim 114, wherein the biological sample is serum or plasma.

116. The method according to any one of claims 101 to 115, wherein each of the egg peptides is coupled to the solid support by a linker-spacer.

117. The method according to claim 116, wherein the linker-spacer comprises a linker chosen from biotin, thiol, hydrazine, and amine, and a spacer chosen from a polypeptide, an oligonucleotide, an alkyl group, and a polyethylene glycol (PEG) group.

118. The method according to claim 117, wherein the spacer is an alkyl group or a PEG group.

119. The method according to claim 118, wherein the alkyl group is a C₁-C₁₈alkyl group.

120. The method according to claim 118, wherein the PEG group is PEG1 to PEG18.

121. The method according to claim 120, wherein the PEG group is PEG12.

122. The method according to any one of claims 116 to 121, wherein the C-terminus of each of the egg peptides is coupled to the solid support by the linker-spacer.

123. The method according to any one of claims 116 to 121, wherein the N-terminus of each of the egg peptides is coupled to the solid support by the linker-spacer.

124. The method according to claim 116, wherein the C-terminus of each of the egg peptides is coupled to the solid support by a biotin-PEG12 linker-spacer.

125. The method according to any one of claims 101 to 124, wherein the solid support is a microsphere bead, glass array, silicone array, membrane, or microtiter plate.

126. The method according to claim 125, wherein the solid support is a microsphere bead.

127. The method according to claim 126, wherein the microsphere bead is an avidin-coupled microsphere bead.

128. The method according to any one of claims 101 to 127, wherein the AAI is IgM, IgA, and/or IgD.

129. The method according to any one of claims 101 to 127, wherein the AAI is IgG and/or IgE.

130. The method according to any one of claims 101 to 127, wherein the AAI is IgE.

131. The method according to any one of claims 101 to 130, wherein the AAI-specific labeling reagent is a detectably labeled anti-human antibody.

132. The method according to claim 131, wherein the detectably labeled anti-human antibody is detectably labeled anti-human IgE antibody.

133. The method according to claim 131 or claim 132, wherein the detectable label of the AAI-specific labeling reagent is chosen from phycoerythrin (PE), a cyanine dye, a fluorescent dye, an infrared dye, a chromogenic dye, an enzyme label, and a radioactive label.

134. The method according to claim 133, wherein the detectable label is PE.

135. The method according to claim 131, wherein the AAI-specific labeling reagent is a PE-labeled anti-human IgE antibody.

136. The method according to any one of claims 101 to 135, wherein the measuring of the binding of the AAI-specific labeling reagent to each AAI-peptide-solid support complex is carried out by a point of care device.

137. The method according to claim 136, wherein the point of care device is a multiplex peptide-bead flow cytometric analysis device or a lateral flow assay device.

138. The method according to any one of claims 101 to 137, wherein measuring the binding of the AAI-specific labeling reagent to each AAI-peptide-solid support complex comprises measuring the mean fluorescent intensity (MFI) of each AAI-specific labeling reagent; and
wherein the AAI-specific labeling reagent is a detectably labeled anti-human antibody.

139. The method according to claim 138, wherein the MFI of each AAI-specific labeling reagent is background subtracted.

140. A method of desensitizing an infant to egg allergens to induce tolerance or non-allergy to eggs comprising administering to the infant a plurality of egg peptides, wherein the plurality of egg peptides comprises up to about 65 egg peptides from 1 to 7 egg proteins,
wherein the egg proteins are selected from ovomucoid, ovalbumin, ovotransferrin, lysozyme, yolk serum albumin, YGP40, and YBG42;
wherein the ovomucoid peptides are selected from: OVM-001 (AEVDCSRFPNATDKE; SEQ ID NO: 1), OVM-003 (RFPNATDKEGKDVLV; SEQ ID NO: 2), OVM-004 (NATDKEGKDVLVCNK; SEQ ID NO: 3), OVM-005 (DKEGKDVLVCNKDLR; SEQ ID NO: 4), OVM-007 (VLVCNKDLRPICGTD; SEQ ID NO: 5), OVM-008 (CNKDLRPICGTDGVT; SEQ ID NO: 6), OVM-009 (DLRPICGTDGVTYTN; SEQ ID NO: 7), OVM-010 (PICGTDGVTYTNDCL; SEQ ID NO: 8), OVM-013 (YTNDCLLCAYSIEFG; SEQ ID NO: 9), OVM-014 (DCLLCAYSIEFGTNI; SEQ ID NO: 10), OVM-015 (LCAYSIEFGTNISKE; SEQ ID NO: 11), OVM-018 (TNISKEHDGECKETV; SEQ ID NO: 12), OVM-021 (ECKETVPMNCSSYAN; SEQ ID NO: 13), OVM-023 (PMNCSSYANTTSEDG; SEQ ID NO: 14), OVM-025 (YANTTSEDGKVMVLC; SEQ ID NO: 15), OVM-027 (EDGKVMVLCNRAFNP; SEQ ID NO: 16), OVM-028 (KVMVLCNRAFNPVCG; SEQ ID NO: 17), OVM-031 (FNPVCGTDGVTYDNE; SEQ ID NO: 18), OVM-032 (VCGTDGVTYDNECLL; SEQ ID NO: 19), OVM-033 (TDGVTYDNECLLCAH; SEQ ID NO: 20), OVM-036 (CLLCAHKVEQGASVD; SEQ ID NO: 21), OVM-038 (KVEQGASVDKRHDGG; SEQ ID NO: 22), OVM-040 (SVDKRHDGGCRKELA; SEQ ID NO: 23), OVM-041 (KRHDGGCRKELAAVS; SEQ ID NO: 24), OVM-042 (DGGCRKELAAVSVDC; SEQ ID NO: 25), OVM-044 (ELAAVSVDCSEYPKP; SEQ ID NO: 26), OVM-046 (VDCSEYPKPDCTAED; SEQ ID NO: 27), OVM-048 (PKPDCTAEDRPLCGS; SEQ ID NO: 28), OVM-050 (AEDRPLCGSDNKTYG; SEQ ID NO: 29), OVM-052 (CGSDNKTYGNKCNFC; SEQ ID NO: 30), OVM-054 (TYGNKCNFCNAVVES; SEQ ID NO: 31), OVM-055 (NKCNFCNAVVESNGT; SEQ ID NO: 32), OVM-056 (NFCNAVVESNGTLTL; SEQ ID NO: 33), and/or OVM-058 (VESNGTLTLSHFGKC; SEQ ID NO: 34);
wherein the ovalbumin peptides are selected from: OVA-001 (MGSIGAASMEFCFDV; SEQ ID NO: 35), OVA-003 (ASMEFCFDVFKELKV; SEQ ID NO: 36), OVA-006 (FKELKVHHANENIFY; SEQ ID NO: 37), OVA-021 (DKLPGFGDSIEAQCG; SEQ ID NO: 38), OVA-028 (SSLRDILNQITKPND; SEQ ID NO: 39), OVA-040 (YLQCVKELYRGGLEP; SEQ ID NO: 40), OVA-053 (IIRNVLQPSSVDSQT; SEQ ID NO: 41), OVA-057 (SQTAXVLVNAIVFKG; SEQ ID NO: 42), OVA-059 (LVNAIVFKGLWEKAF; SEQ ID NO: 43), OVA-064 (KDEDTQAMPFRVTEQ; SEQ ID NO: 44), OVA-068 (TEQESKPVQMMYQIG; SEQ ID NO: 45), OVA-071 (MMYQIGLFRVASMAS; SEQ ID NO: 46), OVA-075 (MASEKMKILELPFAS; SEQ ID NO: 47), OVA-085 (LEQLESIINFEKLTE; SEQ ID NO: 48), OVA-090 (WTSSNVMEERKIKVY; SEQ ID NO: 49), OVA-095 (LPRMKMEEKYNLTSV; SEQ ID NO: 50), OVA-108 (LKISQAVHAAHAEIN; SEQ ID NO: 51), OVA-113 (EAGREVVGSAEAGVD; SEQ ID NO: 52), and/or OVA-120 (EFRADHPFLFCIKHI; SEQ ID NO: 53);
wherein the lysozyme peptides are selected from: LYS-1 (AAAMKRHGLDNYRGY; SEQ ID NO: 54), LYS-2 (GYSLGNWVCAAKFES; SEQ ID NO: 55), LYS-3 (RNTDGSTDYGILQIN; SEQ ID NO: 56), LYS-4 (TDYGILQINSRWWCN; SEQ ID NO: 57), LYS-5 (WCNDGRTPGSRNLCN; SEQ ID NO: 58), and/or LYS-6 (WVAWRNRCKGTDVQA; SEQ ID NO: 59);
wherein the ovotransferrin peptides are selected from: OVT-1 (SSPEEKKCNNLRDLT; SEQ ID NO: 60), OVT-2 (LRDLTQQERISLTCV; SEQ ID NO: 61), OVT-3 (TIEQKLCRQCKGDPK; SEQ ID NO: 62), OVT-4 (DQLTPSPRENRIQWC; SEQ ID NO: 63), and/or OVT-5 (VMDYRECNLAEVPTH; SEQ ID NO: 64);
wherein the serum albumin peptides are selected from: sALB-1 (YQRPASDVICQEYQD SEQ ID NO: 65 or LRDSYGAMADCCSKA SEQ ID NO: 66), sALB-2 (LEKCCKTDNPAECYA; SEQ ID NO: 67), sALB-3 (KQETTPINDNVSQCC; SEQ ID NO: 68), sALB-4 (GVDTKYVPPPFNPDM; SEQ ID NO: 69), and/or sALB-5 (MVDKCCKQSDINTCFGE; SEQ ID NO: 70);
wherein the YGP40 peptides are selected from: YGP40-1 (NYSMPANCYHILVQD; SEQ ID NO: 71), YGP40-2 (VQDCSSELKFLVMMK; SEQ ID NO: 72), YGP40-3 (CAKGCSATKTTPVTV; SEQ ID NO: 73), YGP40-4 (CSATKTTPVTVGFHC; SEQ ID NO: 74), and/or YGP40-5 (FHCLPADSANSLTDK; SEQ ID NO: 75); and
wherein the YBG42 peptides are selected from: YGP42-1 (VDHQSLSREVHINTS; SEQ ID NO: 76), YGP42-2 (GVCGNNDREKHNELL; SEQ ID NO: 77), and/or YGP42-3 (SSDKKSASEDVVESV; SEQ ID NO: 78);
wherein the amino acid sequence of any one or more of the egg peptides can have one to four conservative amino acid substitutions therein.

141. The method according to claim 140, wherein the ovomucoid peptides are selected from: OVM-014, OVM-004, OVM-009, OVM-032, and/or OVM-031.

142. The method according to claim 140, wherein the ovomucoid peptides are selected from: OVM-014, OVM-004, OVM-009, OVM-032, and/or OVM-013.

143. The method according to any one of claims 101 to 103, wherein the ovalbumin peptides are selected from: OVA-064, OVA-108, and/or OVA-028.

144. The method according to any one of claims 140 to 143, wherein the lysozyme peptides are selected from: LYS-1, LYS-3, and/or LYS-6.

145. The method according to any one of claims 140 to 144, wherein the ovotransferrin peptides are selected from: OVT-1, OVT-3, and/or OVT-5.

146. The method according to any one of claims 140 to 145, wherein the serum albumin peptides are selected from: sALB-1, sALB-3 and/or sALB-4.

147. The method according to any one of claims 140 to 146, wherein the YGP40 peptides are YGP40-1 and/or YGP40-5.

148. The method according to any one of claims 140 to 147, wherein the YBG42 peptide is YGP42-1.

149. The method according to claim 140, wherein the composition comprises the egg peptides: OVM-001, OVM-003, OVM-004, OVM-005, OVM-007, OVM-008, OVM-009, OVM-010, OVM-013, OVM-014, OVM-015, OVM-018, OVM-021, OVM-023, OVM-025, OVM-027, OVM-028, OVM-031, OVM-032, OVM-033, OVM-036, OVM-038, OVM-040, OVM-041, OVM-042, OVM-044, OVM-046, OVM-048, OVM-050, OVM-052, OVM-054, OVM-055, OVM-056, OVM-058, OVA-001, OVA-003, OVA-006, OVA-021, OVA-028, OVA-040, OVA-053, OVA-057, OVA-059, OVA-064, OVA-068, OVA-071, OVA-075, OVA-085, OVA-090, OVA-095, OVA-108, OVA-113, OVA-120, OVT-1, OVT-3, OVT-5, LYS-1, LYS-3, LYS-6, sALB-1, sALB-4, sALB-5, YGP40-1, YGP40-5, and YGP42-1.

150. The method according to claim 140, wherein the composition comprises the egg peptides: OVT-1, OVT-2, OVT-3, OVT-4, OVT-5, LYS-1, LYS-2, LYS-3, LYS-4, LYS-5, LYS-6, sALB-1, sALB-2, sALB-3, sALB-4, sALB-5, YGP42-1, YGP42-2, YGP42-3, YGP40-1, YGP40-2, YGP40-3, YGP40-4, and YGP40-5.

151. The method according to claim 140, wherein the composition comprises the egg peptides: OVM-014, OVM-004, OVM-009, OVM-032, OVA-064, OVA-108, OVM-013, OVA-028, OVM-031, LYS-1, and LYS-3.

152. The method according to claim 140, wherein the composition comprises the egg peptides: OVM-014, OVM-004, OVM-009, OVM-032, OVA-064, OVA-108, OVM-013, and OVA-028.
